(19) United States
(12) Patent Application Publication
(10) Pub. No.: US 2021/0380701 A1
(43) Pub. Date: Dec. 9, 2021

(54) MULTIVALENT IgM- AND IgA-Fc-BASED BINDING MOLECULES

(71) Applicant: IGM Biosciences, Inc., Mountain View, CA (US)

(72) Inventors: Ramesh BALIGA, Redwood City, CA (US); Paul HINTON, Sunnyvale, CA (US); Dean NG, San Francisco, CA (US); Bruce KEYT, Hillsborough, CA (US)

(21) Appl. No.: 17/287,828

(22) PCT Filed: Oct. 23, 2019
(86) PCT No.: PCT/US2019/057702

§ 371 Date: Apr. 22, 2021

Related U.S. Application Data
(60) Provisional application No. 62/749,429, filed on Oct. 23, 2018.

(51) Int. Cl. C07K 16/28

(52) U.S. Cl. CPC C07K 16/2827; C07K 2317/528; C07K 2317/31; C07K 2317/734; C07K 2317/622

(57) ABSTRACT

This disclosure provides IgM- and IgA-derived binding molecules comprising binding polypeptides, e.g., receptor ectodomains, ligands, or receptor-binding fragments thereof, and the like, fused to multimerizing IgM or IgA constant regions.

### CROSS-REFERENCE TO RELATED APPLICATIONS

This application claims the benefit of U.S. Provisional Patent Application Ser. No. 62/749,429, filed Oct. 23, 2018, which is incorporated herein by reference in its entirety.

### SEQUENCE LISTING

The instant application contains a Sequence Listing which has been submitted electronically in ASCII format and is hereby incorporated by reference in its entirety. The ASCII copy was created on Oct. 23, 2019, is named 09789-021WO1-Sequence-Listing, and is 190,396 bytes in size.

### BACKGROUND

Antibodies and antibody-like molecules that can multimerize, such as IgA and IgM antibodies, have emerged as promising drug candidates in the fields of, e.g., immuno-oncology and infectious diseases allowing for improved specificity, improved avidity, and the ability to bind to multiple binding targets. See, e.g., U.S. Pat. Nos. 9,951,134, 10,400,038, and 9,938,347, U.S. Patent Application Publication Nos. US20190100597A1, US20180118814A1, US20180118816A1, US20190185570A1, and US20180265596A1, and PCT Publication Nos. WO 2018/017888, WO 2018/017763, WO 2018/017889, WO 2018/017761, and WO 2019/165340, the contents of which are incorporated herein by reference in their entireties.

The Fc region of IgG has long been used as a fusion partner for therapeutic polypeptides. The first Fc fusion protein described was a CD4-Fc fusion for use in blocking entry of HIV into cells (Capon, D J, et al., Nature 337:515-531 (1989)). Fusion of therapeutic proteins to IgG Fc stabilizes and extends the half-life of the therapeutic polypeptide, as well as providing IgG-specific effector functions (Czajkowsky, D M, et al., EMBO Mol. Med. 4:1015-1028 (2012)). Starting with etanercept, a dimeric IgG1-fc-human TNF receptor fusion approved by the FDA in 1998, a wide variety of Fc fusion proteins are now on the market as therapeutics (see, e.g., Czajkowsky et al., Table 1). IgG fusions, however, a limited as an IgG fusion protein can only be expressed as a monomer or a dimer, limiting efficacy in some situations. Indeed, monomeric forms of the TNF receptor Fc fusion protein had greatly reduced TNFα inhibitory activity as compared to the dimer (Pepel, K., et al, J. Exp. Med. 174:1483-1489 (1991)).

Many reports have described polymerized therapeutic protein-Fc fusions through a variety of methods. In one report, IgG Fc regions were engineered to facilitate hexamerization of a malaria antigen-IgG fusion, but effector function of the IgG fusion portions were altered, and the molecules were not immunogenic when used to immunize animals, in contrast to the corresponding monomeric fusion proteins (Mekhaiel, D N, et al., Scientific Reports 1, Doi: 10.1038/srep00124 (2011)). In another study, the human PD-L1 ectodomain was fused to wild-type human IgM constant region and expressed either with or without human J-chain and was tested in in vitro flow cytometry and plate-based immunoassays, but the ability of the constructs to induce signal transduction in PD-1-expressing cells was not tested (Ammann, J U., et al, Eur. J. Immunol 42:1354-1356 (2012).

There remains a need for higher avidity Fc fusion therapeutics that maintain the stability and serum half-life characteristics of IgG Fc fusion proteins.

### SUMMARY

This disclosure provides a multimeric binding molecule that includes two, five, or six bivalent binding units or variants or fragments thereof where each binding unit includes two IgA or IgM heavy chain constant regions or multimerizing fragments or variants thereof, each fused to a binding polypeptide or fragment thereof that specifically binds to a binding partner expressed on the surface of a cell, where the binding polypeptide is not an antibody or antigen-binding fragment of an antibody, and where binding of the binding polypeptide to the binding partner modulates signal transduction in the cell. In certain embodiments at least three, at least four, at least five, at least six, at least seven, at least eight, at least nine, at least ten, at least eleven or twelve of the binding polypeptides bind to and modulate signal transduction of the same binding partner. Moreover, in certain embodiments the binding molecule can induce or inhibit signal transduction in the cell at a higher potency than an equivalent amount of a monovalent or divalent binding molecule with one or two binding polypeptides binding to the same binding partner.

This disclosure further provides a multimeric binding molecule that includes two, five, or six bivalent binding units or variants or fragments thereof where each binding unit includes two IgA or IgM heavy chain constant regions or multimerizing fragments or variants thereof, each fused to binding polypeptide, where at least three of the binding polypeptides include a receptor ectodomain that specifically binds to a binding partner that includes a ligand or receptor-binding fragment thereof, where the receptor ectodomain is not an antibody or antigen-binding fragment of an antibody, and where binding of the receptor ectodomain to the ligand can modulate signal transduction in a cell that expresses the receptor. In certain embodiments at least three, at least four, at least five, at least six, at least seven, at least eight, at least nine, at least ten, at least eleven or twelve of the receptor ectodomains bind to the same ligand. Moreover, in certain embodiments the binding molecule can modulate signal transduction at a higher potency than an equivalent amount of a monomeric or dimeric binding molecule with one or two receptor ectodomains binding to the same ligand.

In certain of the binding multimeric binding molecules provided by the disclosure, each binding unit includes two IgA heavy chain constant regions or multimerizing fragments or variants thereof that each include an IgA Cα3 domain and an IgA tailpiece domain and where the multimeric binding molecule further includes a J-chain or functional fragment or variant thereof. In certain embodiments, each IgA heavy chain constant region or multimerizing fragment or variant thereof further includes an IgA Cα2 domain situated N-terminal to the IgA Cα3 and IgA tailpiece domains. For example, the heavy chain constant regions of the multimeric binding molecule can include amino acids 125 to 353 of SEQ ID NO: 24, or amino acids 113 to 340 of SEQ ID NO: 25. In certain embodiments, each IgA heavy chain constant region or multimerizing fragment or variant thereof further includes an IgA hinge region situated N-terminal to the IgA Cα2 domain. For example, the heavy chain constant regions of the multimeric binding molecule can include amino acids 102 to 353 of SEQ ID NO: 24, or amino acids 102 to 340 of SEQ ID NO: 25.

In certain of the binding multimeric binding molecules provided by the disclosure, each binding unit includes two IgM heavy chain constant regions or multimerizing fragments or variants thereof that each include an IgM Cμ4 domain and an IgM tailpiece domain. In certain embodiments, each IgM heavy chain constant region or multimerizing fragment or variant thereof further includes an IgM Cμ3 domain situated N-terminal to the IgM Cμ4 and IgM tailpiece domains. In certain embodiments, each IgM heavy chain constant region or multimerizing fragment or variant thereof further includes an IgM Cμ2 domain situated N-terminal to the IgM Cμ3 domain. For example, the heavy chain constant regions of the multimeric binding molecule can include the amino acid sequence SEQ ID NO: 3. In other embodiments, each IgM heavy chain constant region or multimerizing fragment or variant thereof includes the amino acid sequence SEQ ID NO: 4, which confers upon the multimeric binding molecule reduced complement-dependent cytotoxicity (CDC) activity relative to a corresponding binding molecule that includes the wild type multimerizing fragment of the human IgM constant region of SEQ ID NO: 3. In certain embodiments where each IgM heavy chain constant region or multimerizing fragment or variant thereof includes an IgM Cμ3 domain situated N-terminal to the IgM Cμ4 and IgM tailpiece domains, each IgM heavy chain constant region or multimerizing fragment or variant thereof further includes an IgG hinge region or functional variant thereof situated N-terminal to the IgM Cμ3 domain. In certain embodiments the IgG hinge region is a variant human IgG1 hinge region fused to a multimerizing fragment of the human IgM constant region that includes the Cμ3, Cμ4, and TP domains. For example, the multimerizing hinge-IgM constant region fragment can include the amino acid sequence SEQ ID NO: 6, or the amino acid sequence SEQ ID NO: 7, the latter sequence including a Cμ3 region that confers the binding molecules with reduced CDC activity relative to a corresponding binding molecule that includes the multimerizing hinge-IgM fragment of SEQ ID NO: 6.

In certain embodiments an IgM-Fc-based multimeric binding molecule provided by this disclosure is pentameric and further includes a J-chain or functional fragment or variant thereof. The J-chain or functional fragment or variant thereof is a variant J-chain can include one or more single amino acid substitutions, deletions, or insertions relative to a wild-type J-chain that can, e.g., affect serum half-life of the multimeric binding molecule. For example, in certain embodiments, the multimeric binding molecule exhibits an increased serum half-life upon administration to an animal relative to a reference multimeric binding molecule that is identical except for the one or more single amino acid substitutions, deletions, or insertions, and is administered in the same way to the same animal species. In certain embodiments, the J-chain or functional fragment or variant thereof includes an amino acid substitution at the amino acid position corresponding to amino acid Y102 of the wild-type human J-chain (SEQ ID NO: 15). In certain embodiments, the amino acid corresponding to Y102 of SEQ ID NO: 15 is substituted with alanine (A), serine (S), or arginine (R). In certain embodiments, the amino acid corresponding to Y102 of SEQ ID NO: 15 is substituted with alanine (A). In certain embodiments, the J-chain is a variant human J-chain that includes the amino acid sequence SEQ ID NO: 16. In certain embodiments the J-chain or functional fragment thereof includes an amino acid substitution at the amino acid position corresponding to amino acid N49, amino acid S51, or both N49 and S51 of the human J-chain (SEQ ID NO: 15), provided that the single amino acid substitution corresponding to position S51 of SEQ ID NO: 15 is not a threonine (T) substitution. In certain embodiments, the position corresponding to N49 of SEQ ID NO: 15 is substituted with alanine (A), glycine (G), threonine (T), serine (S) or aspartic acid (D). In certain embodiments, the position corresponding to N49 of SEQ ID NO: 15 is substituted with alanine (A). In certain embodiments, the J-chain is a variant human J-chain and includes the amino acid sequence SEQ ID NO: 17. In certain embodiments, the position corresponding to S51 of SEQ ID NO: 15 is substituted with alanine (A) or glycine (G). In certain embodiments, the position corresponding to S51 of SEQ ID NO: 15 is substituted with alanine (A). In certain embodiments, the J-chain is a variant human J-chain and includes the amino acid sequence SEQ ID NO: 18.

In certain embodiments where a multimeric binding molecule provided by this disclosure includes a J-chain or functional fragment or variant thereof, the J-chain or functional fragment or variant thereof can further include a heterologous polypeptide, where the heterologous polypeptide is directly or indirectly fused to the J-chain or functional fragment or variant thereof, e.g., via a peptide linker that can include at least 5 amino acids, but no more than 25 amino acids. In certain embodiments the peptide linker consists of GGGGS (SEQ ID NO: 19), GGGGSGGGGS (SEQ ID NO: 20), GGGGSGGGGSGGGGS (SEQ ID NO: 21), GGGGSGGGGSGGGGSGGGGS (SEQ ID NO: 22), or GGGGSGGGGSGGGGSGGGGSGGGGS (SEQ ID NO: 23). In certain embodiments, the heterologous polypeptide can be fused to the N-terminus of the J-chain or fragment or variant thereof, the C-terminus of the J-chain or fragment or variant thereof, or identical or non-identical heterologous polypeptides can be to both the N-terminus and C-terminus of the J-chain or fragment or variant thereof. In certain embodiments, the heterologous polypeptide can influence the absorption, distribution, metabolism and/or excretion (ADME) of the multimeric binding molecule. In certain embodiments, the heterologous polypeptide can include an antigen binding domain, e.g., an antibody or antigen-binding fragment thereof, where the antigen-binding fragment can be a Fab fragment, a Fab′ fragment, an F(ab′)2 fragment, an Fd fragment, an Fv fragment, a single-chain Fv (scFv) fragment, a disulfide-linked Fv (sdFv) fragment, or any combination thereof. In certain embodiments, the antigen-binding fragment is a scFv fragment.

In certain embodiments an IgA-Fc-based binding molecule as provide herein can include four identical binding polypeptides. In certain embodiments an IgM-Fc-based binding molecule as provided herein can be pentameric and can include ten identical binding polypeptides. In certain embodiments an IgM-Fc-based binding molecule as provided herein can be pentameric and can include twelve identical binding polypeptides.

In certain of the binding multimeric binding molecules provided by the disclosure, each binding polypeptide is a ligand or receptor-binding fragment thereof, a cytokine or receptor-binding fragment thereof, a growth factor or receptor binding fragment thereof, a neurotransmitter or receptor binding fragment thereof, a peptide or protein hormone or receptor binding fragment thereof, an immune checkpoint modulator ligand or receptor-binding fragment thereof, or a receptor-binding fragment of an extracellular matrix protein. The ligand or receptor-binding fragment thereof can include, but is not limited to, a chemokine, a complement protein, a fibroblast growth factor (FGF) family ligand, an immune checkpoint modulator ligand, an epidermal growth factor (EGF), an interferon, a tumor necrosis factor superfamily (TNFSF) ligand, a vascular endothelial growth factor (VEGF) family ligand, a transforming growth factor-β superfamily (TGFβsf) ligand, any receptor-binding fragment thereof, or any combination thereof. In certain embodiments where the binding polypeptide includes a TNFSF ligand or receptor-binding fragment thereof, the TNFSF ligand can include, but is not limited to, TRAIL, OX40 ligand, CD40 ligand, a glucocorticoid-induced tumor necrosis factor receptor ligand (GITRL), 4-1BB ligand, any receptor binding fragment thereof, or any combination thereof. In certain embodiments where the binding polypeptide includes an immune checkpoint modulator ligand protein or receptor-binding fragment thereof, the immune checkpoint modulator protein can include CD86 or a receptor-binding fragment thereof, CD80 or a receptor-binding fragment thereof, PD-L1 or a receptor-binding fragment thereof, or any combination thereof.

In an exemplary embodiment, the binding polypeptide includes a receptor-binding fragment of human PD-L1, e.g., amino acids 19 to 127 of SEQ ID NO: 8, which contains the V-type domain of human PD-L1, or SEQ ID NO: 9, which contains the V-type and C2-type domains of human PD-L1. In an exemplary embodiment, a multimeric binding molecule provided by the disclosure includes ten or twelve copies of a polypeptide including the amino acid sequence SEQ ID NO: 11 or SEQ ID NO: 13 and can further include a variant J-chain including the amino acid sequence SEQ ID NO: 16. A binding molecule according to this exemplary embodiment can be an agonist of PD-1.

In certain embodiments, the binding partner can be a cell-surface receptor protein or an immune checkpoint modulator.

In certain embodiments where the binding polypeptide includes a receptor ectodomain, the binding polypeptide can include, but is not limited to, a ligand-binding fragment of a tumor necrosis factor superfamily receptor (TNFrSF), a ligand-binding fragment of an immune checkpoint modulator receptor, ligand-binding fragment of a TGFβ receptor, or any combination thereof. For example, a TNFrSF receptor fragment can include, but is not limited to, a ligand-binding fragment of death domain containing receptor-4 (DR4), death domain containing receptor-5 (DR5), OX-40, CD40, 4-1BB, glucocorticoid-induced tumor necrosis factor receptor (GITR), or any combination thereof. As a further example, an immune checkpoint modulator receptor ectodomain can include, but is not limited to a ligand-binding fragment of PD-1, a ligand-binding fragment of CTLA4, a ligand-binding fragment of LAG3, a ligand-binding fragment of CD28, a ligand-binding fragment of immunoglobulin-like domain containing receptor 2 (ILDR2), a ligand-binding fragment of T-cell immunoglobulin mucin family member 3 (TIM-3), or any combination thereof. As a further example, a TGFβ receptor can include, but is not limited to a ligand binding fragment of a TGFβR-1, a TGFβR-2, a TGFβR3, or any combination thereof.

The disclosure further provides an isolated polynucleotide that includes a nucleic acid sequence that encodes a subunit of a multimeric binding molecule as provided herein, where each subunit includes an IgA or IgM heavy chain constant region or multimerizing fragment or variant thereof fused to a binding polypeptide or fragment thereof that specifically binds to a binding partner, or a receptor ectodomain that specifically binds to a ligand. Further disclosed is a vector that includes the provided polynucleotide, and a host cell that includes the provided vector or polynucleotide. In certain embodiments, the provided host cell can further include an isolated polynucleotide that includes a nucleic acid sequence encoding a J-chain or functional fragment or variant thereof as provided by the disclosure.

The disclosure further provides a method for treating an autoimmune disorder, an inflammatory disorder, or a combination thereof in a subject in need of treatment where the method includes administering to the subject an effective amount of a multimeric binding molecule as provided herein, where the multimeric binding molecule exhibits greater potency than an equivalent amount of a monomeric or dimeric binding molecule binding to the same binding partner.

The disclosure further provides a method for preventing transplantation rejection in a transplantation recipient, where the method includes administering to the subject an effective amount of the multimeric binding molecule as provided herein, where the multimeric binding molecule exhibits greater potency than an equivalent amount of a monomeric or dimeric binding molecule binding to the same binding partner.

### BRIEF DESCRIPTION OF THE DRAWINGS/FIGURES

FIG. 1A shows a prototype hexameric binding molecule that has six IgM-derived binding units each having two IgM-derived heavy chain fragments that include a Cμ2 domain, a Cμ3 domain, and a Cμ4-tp domain, where the IgM derived heavy chain fragments are fused to the C-terminus of a ligand or receptor binding polypeptide.

FIG. 1B shows a prototype pentameric binding molecule that has five IgM-derived binding units each having two IgM-derived heavy chain fragments that include a Cμ2 domain, a Cμ3 domain, and a Cμ4-tp domain, where the IgM derived heavy chain fragments are fused to the C-terminus of binding polypeptide, and where the pentameric binding molecule further includes a modified J-chain bearing optional N- and C-terminal fusions of heterologous polypeptides, e.g., scFv antibody binding domains.

FIG. 2 shows a prototype pentameric binding molecule has five IgM-derived binding units each having two IgM-derived heavy chain fragments that have a Cμ2 domain, a Cμ3 domain, and a Cμ4-tp domain, where the IgM derived heavy chain fragments are fused to the C-terminus of binding polypeptide, where the binding polypeptides are receptor ectodomains, and where the pentameric binding molecule further includes a modified J-chain bearing optional N- and C-terminal fusions of heterologous polypeptides, e.g., scFv antibody binding domains.

FIG. 3A-C shows the structures of the PD-L1 binding molecules produced according to Example 1. FIG. 3A: PD-L1-IgM; FIG. 3B: PD-L1-H-IgM; FIG. 3C: PD-L1-Fc.

FIG. 4 is a graph showing the ability of various PD-L1 binding molecules to stimulate PD-1 activity in reporter Jurkat T-cells.

### DETAILED DESCRIPTION

### Definitions

The term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino (“N”) to carboxy (“C”) orientation. The headings provided herein are not limitations of the various embodiments or embodiments of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt many different conformations and are referred to as unfolded. As used herein, the term glycoprotein refers to a protein coupled to at least one carbohydrate moiety that is attached to the protein via an oxygen-containing or a nitrogen-containing side chain of an amino acid, e.g., a serine or an asparagine.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

As used herein, the term “a non-naturally occurring polypeptide” or any grammatical variants thereof, is a conditional definition that explicitly excludes, but only excludes, those forms of the polypeptide that are, or could be, determined or interpreted by a judge or an administrative or judicial body, to be “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides which retain at least some of the properties of the corresponding native polypeptide, for example, specifically binding to a binding partner. Fragments of polypeptides include, for example, proteolytic fragments, as well as deletion fragments. Variants of, e.g., a polypeptide include fragments as described above, and also polypeptides with altered amino acid sequences due to amino acid substitutions, deletions, or insertions. In certain embodiments, variants can be non-naturally occurring. Non-naturally occurring variants can be produced using art-known mutagenesis techniques. Variant polypeptides can include conservative or non-conservative amino acid substitutions, deletions or additions. Derivatives are polypeptides that have been altered so as to exhibit additional features not found on the original polypeptide. Examples include fusion proteins or chemical conjugates. Variant polypeptides can also be referred to herein as “polypeptide analogs.” As used herein a “derivative” of a polypeptide can also refer to a subject polypeptide having one or more amino acids chemically derivatized by reaction of a functional side group. Also included as “derivatives” are those peptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides and binding molecules of the present disclosure do not abrogate the binding of the polypeptide or binding molecule containing the amino acid sequence, to a binding partner to which the binding molecule binds. Methods of identifying nucleotide and amino acid conservative substitutions which do not eliminate binding partner-binding are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94:412-417 (1997)).

The term “polynucleotide” is intended to encompass a singular nucleic acid as well as plural nucleic acids and refers to an isolated nucleic acid molecule or construct, e.g., messenger RNA (mRNA), cDNA, or plasmid DNA (pDNA). A polynucleotide can include a conventional phosphodiester bond or a non-conventional bond (e.g., an amide bond, such as found in peptide nucleic acids (PNA)). The terms “nucleic acid” or “nucleic acid sequence” refer to any one or more nucleic acid segments, e.g., DNA or RNA fragments, present in a polynucleotide.

By an “isolated” nucleic acid or polynucleotide is intended any form of the nucleic acid or polynucleotide that is separated from its native environment. For example, gel-purified polynucleotide, or a recombinant polynucleotide encoding a polypeptide contained in a vector would be considered to be “isolated.” Also, a polynucleotide segment, e.g., a PCR product, which has been engineered to have restriction sites for cloning is considered to be “isolated.” Further examples of an isolated polynucleotide include recombinant polynucleotides maintained in heterologous host cells or purified (partially or substantially) polynucleotides in a non-native solution such as a buffer or saline. Isolated RNA molecules include in vivo or in vitro RNA transcripts of polynucleotides, where the transcript is not one that would be found in nature. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, the term “a non-naturally occurring polynucleotide” or any grammatical variants thereof, is a conditional definition that explicitly excludes, but only excludes, those forms of the nucleic acid or polynucleotide that are, or could be, determined or interpreted by a judge, or an administrative or judicial body, to be “naturally-occurring.”

As used herein, a “coding region” is a portion of nucleic acid which consists of codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can include two or more coding regions. In addition, a vector, polynucleotide, or nucleic acid can include heterologous coding regions, either fused or unfused to another coding region. Heterologous coding regions include without limitation, those encoding specialized elements or motifs, such as a secretory signal peptide or a heterologous functional domain.

In certain embodiments, the polynucleotide or nucleic acid is DNA. In the case of DNA, a polynucleotide including a nucleic acid which encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions that function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA), transfer RNA, or ribosomal RNA.

Polynucleotide and nucleic acid coding regions can be associated with additional coding regions which encode secretory or signal peptides, which direct the secretion of a polypeptide encoded by a polynucleotide as disclosed herein. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells can have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse β-glucuronidase.

Disclosed herein are certain binding molecules, or binding-partner-binding fragments, variants, or derivatives thereof. As used herein, the term “binding molecule” refers in its broadest sense to a molecule that includes a “binding polypeptide,” or two, three, four, five, six, seven, eight, nine, ten, eleven, or twelve “binding polypeptides” that specifically binds to a “binding partner” target or molecular determinant, or two or more “binding partner” targets or molecular determinants. As described further herein, a binding molecule can include one or more “binding polypeptides” or a fragment thereof, as described herein. A non-limiting example of a binding molecule is an antibody or fragment thereof that retains antigen-specific binding. But according to certain embodiments of the present disclosure, the one or more “binding polypeptides” of the binding molecule are not antibodies or antigen-binding domains derived from antibodies. That is, the binding polypeptides do not include antigen-binding domains, e.g., a VH and/or a VL, of an antibody molecule.

As used herein, the term “binding polypeptide” refers to a region of a binding molecule, situated N-terminal to an IgM or IgA constant region or multimerizing fragment thereof, that is sufficient to specifically bind to a binding partner, e.g., a receptor expressed on the surface of a cell, or where the binding polypeptide includes a receptor ectodomain, a portion that is sufficient to specifically bind to a ligand binding partner. For example, a ligand such as PD-L1, or a receptor-binding fragment thereof, that specifically binds to the receptor PD-1, is a “binding polypeptide” as defined herein, whereas PD-1 is defined, relative to PD-L1, as the “binding partner,” expressed on a cell, that the PD-L1 binding polypeptide binds to.

The term “immunoglobulin” as used herein refers to polypeptide that is, or is derived from an immunoglobulin molecule, and includes a portion of a binding molecule as provided herein. This disclosure provides binding molecules that are not traditional “antibodies,” in that they do not include the typical antibody antigen-binding domains of an antibody but do include certain immunoglobulin constant region domains that allow the binding molecules provided herein to readily multimerize into dimers, pentamers, or hexamers. Basic immunoglobulin structures in vertebrate systems are relatively well understood. (See, e.g., Harlow et al., Antibodies: A Laboratory Manual, Cold Spring Harbor Laboratory Press, 2nd ed. 1988).

As will be discussed in more detail below, the term “immunoglobulin” includes various broad classes of polypeptides that can be distinguished biochemically. Only a subset of immunoglobulin polypeptides have the ability to multimerize. Those skilled in the art will appreciate that heavy chains are classified as gamma, mu, alpha, delta, or epsilon, (γ, μ, α, δ, ε) with some subclasses among them (e.g., γ1-γ4 or α1-α2)). It is the nature of this chain that determines the “isotype” of the antibody as IgG, IgM, IgA IgG, or IgE, respectively. The immunoglobulin subclasses (subtypes) e.g., IgG₁, IgG₂, IgG₃, IgG₄, IgA₁, IgA₂, are well characterized and are known to confer functional specialization. Modified versions of each of these immunoglobulins are readily discernible to the skilled artisan in view of the instant disclosure and, accordingly, are within the scope of this disclosure. In certain embodiments, this disclosure provides modified human IgM constant regions.

Light chains are classified as either kappa or lambda (κ, λ), and are optional or unnecessary in the binding molecules provided herein. Each heavy chain class can be bound with either a kappa or lambda light chain. In general, the light chains, if present, and heavy chains are covalently bonded to each other, and the “tail” portions of the two heavy chains are bonded to each other by covalent disulfide linkages or non-covalent linkages when the immunoglobulins are expressed. In the heavy chain, the amino acid sequences run from an N-terminus at the forked ends of the Y configuration to the C-terminus at the bottom of each chain. The basic subunit structure of a multimeric binding molecule as provided herein, e.g., two IgM heavy chains each fused to the C-terminus of a binding polypeptide, includes two heavy chain subunits covalently connected via disulfide bonds to form a “Y” structure, also referred to herein as a “binding unit.”

The term “binding unit” is used herein to refer to the portion of a binding molecule that corresponds to a standard immunoglobulin structure, e.g., an antibody-like molecule, and antibody-derived molecule, a binding partner-binding fragment thereof, or multimerizing fragment thereof, which corresponds to the standard “H2L2” immunoglobulin structure, i.e., two heavy chains or fragments thereof, which can further include two light chains or fragments thereof. In certain embodiments, e.g., where the binding molecule is an IgG immunoglobulin, the terms “binding molecule” and “binding unit” are equivalent. In other embodiments, e.g., where the binding molecule is multimeric, e.g., a dimeric IgA immunoglobulin derived molecule, a pentameric IgM immunoglobulin derived molecule, or a hexameric IgM immunoglobulin derived molecule, the binding molecule includes two, four, or five “binding units.” A binding unit need not include full-length immunoglobulin heavy chain, but in the multimeric binding molecules provided herein, each binding unit will include sufficient portions of an IgA or IgM immunoglobulin constant region to allow multimerization (“a multimerizing fragment”). Certain IgM-derived binding molecules provided in this disclosure are pentameric or hexameric and include five or six bivalent binding units that include IgM constant regions, e.g., modified human IgM constant regions, or “multimerizing fragments thereof,” i.e., at least the Cμ4 and tailpiece regions of the IgM constant region. As used herein, a binding molecule that includes two or more binding units, e.g., two, five, or six binding units, is referred to as “multimeric.”

The term “J-chain” as used herein refers to the J-chain of native sequence IgM or IgA antibodies of any animal species, any functional fragment thereof, derivative thereof, and/or variant thereof, including a mature human J-chain amino acid sequence provided herein as SEQ ID NO: 15. Various J-chain variants and modified J-chain derivatives are disclosed herein. As persons of ordinary skill in the art will recognize, a “functional fragment” or “functional variant” includes those fragments and variants that can associate with IgM heavy chain constant regions to form a pentameric IgM-derived binding molecule or a dimeric IgA binding molecule, and/or can associate with certain immunoglobulin receptors on cells such as the polymeric Ig receptor (PIgR).

The term “variant J-chain” is used herein to refer to a J-chain that includes amino acid substitutions, deletions, or insertions that alter a physical or physiological property of the polypeptide. For example, certain variant J-chain amino acid sequences are provided herein that alter the glycosylation pattern of the J-chain, or that increase the serum half-life of an IgM binding molecule that includes the variant J-chain. Exemplary variant J-chains are provided, e.g., in PCT Publication No. WO 2019/169314, the contents of which is incorporated herein by reference in its entirety.

The term “modified J-chain” is used herein to refer to a J-chain polypeptide that includes a heterologous moiety, e.g., a heterologous polypeptide, e.g., an extraneous binding domain, introduced into the native sequence. The introduction can be achieved by any means, including direct or indirect fusion of the heterologous polypeptide or other moiety or by attachment through a peptide or chemical linker. The term “modified human J-chain” encompasses, without limitation, a native sequence mature human J-chain of the amino acid sequence of SEQ ID NO: 15 or functional fragment thereof modified by the addition of a heterologous moiety, e.g., a heterologous polypeptide, e.g., an extraneous binding domain. In certain embodiments the heterologous moiety does not interfere with efficient polymerization of IgM into a pentamer and binding of such polymers to a target. Exemplary modified J-chains can be found, e.g., in U.S. Pat. Nos. 9,951,134 and 10,400,038, in U.S. Patent Application Publication Nos. US-2019-0185570 and US-2018-0265596, each of which is incorporated herein by reference in its entirety.

As used herein the term “IgM-derived binding molecule” refers collectively to native IgM antibodies, IgM-like antibodies, as well as other IgM-derived binding molecules comprising non-antibody binding and/or functional domains instead of an antibody antigen binding domain or subunit thereof, and any fragments, e.g., multimerizing fragments, variants, or derivatives thereof.

As used herein, the term “IgM-like binding molecule” refers generally to a variant antibody-derived binding molecule that still retains the ability to form hexamers, or in association with J-chain, form pentamers. An IgM-like binding molecule or other IgM-derived binding molecule typically includes at least the Cμ4-tp domains of the IgM constant region but can include heavy chain constant region domains from other antibody isotypes, e.g., IgG, from the same species or from a different species. An IgM-like binding molecule or other IgM-derived binding molecule can likewise be an fragment in which one or more constant region domains are deleted, as long as the IgM-like antibody is capable of forming hexamers and/or pentamers. Thus, an IgM-like binding molecule or other IgM-derived binding molecule can be, e.g., a hybrid IgM/IgG antibody or can be a “multimerizing fragment” of an IgM-derived binding molecule.

The terms “valency,” “bivalent,” “multivalent” and grammatical equivalents, refer to the number of binding polypeptide domains in given binding molecule or binding unit as provided herein. As such, the terms “bivalent”, “tetravalent”, and “hexavalent” in reference to a given binding molecule, e.g., an IgM-derived binding molecule or fragment thereof, denote the presence of two binding polypeptides, four binding polypeptides, and six binding polypeptides, respectively. In a typical IgM-derived binding molecule where each binding unit is bivalent, the binding molecule itself can have 10 or 12 valencies. A bivalent or multivalent binding molecule can be monospecific, i.e., all of the binding polypeptides are the same, or can be bispecific or multispecific, e.g., where two or more binding polypeptides are different, e.g., bind to different epitopes on the same binding partner, or bind to entirely different binding partners.

The term “epitope” as used herein includes any molecular determinant on a binding partner capable of specific binding to a binding polypeptide as defined herein. In certain embodiments, an epitope can include chemically active surface groupings of molecules such as amino acids, sugar side chains, phosphoryl, or sulfonyl, and, in certain embodiments, can have three-dimensional structural characteristics, and or specific charge characteristics.

The term “binding partner” is used in the broadest sense to be a target of a binding polypeptide as provided herein and includes substances that can be bound by a binding molecule as provided herein. A binding partner can be, e.g., a polypeptide, a nucleic acid, a carbohydrate, a lipid, or other molecule. In certain embodiments the binding partner is a receptor or other moiety expressed or present on the surface of a cell. In other embodiments, where the binding polypeptide is a receptor ectodomain, the binding partner can be a soluble or cell-bound ligand, or receptor-binding fragment thereof. Moreover, a “binding partner” can, for example, be a cell, an organ, or an organism, e.g., an animal, plant, microbe, or virus, that includes an epitope that can be bound by a binding molecule or binding polypeptide as provided herein.

Both the light and heavy chains of immunoglobulins are divided into regions or “domains” of structural and functional homology. For example, the constant region domains of an IgM heavy chain (e.g., CH1 or Cμ1, CH2 or Cμ2, CH3 or Cμ3, CH4 or Cμ4, or tailpiece) confer biological properties such as secretion, transplacental mobility, Fc receptor binding, complement binding, ability to multimerize, and the like. By convention the numbering of the constant region domains increases as they become more distal from the amino-terminus of the typical immunoglobulin.

The Kabat numbering system for the human IgM constant domain can be found in Kabat, et. al. “Tabulation and Analysis of Amino acid and nucleic acid Sequences of Precursors, V-Regions, C-Regions, J-Chain, T-Cell Receptors for Antigen, T-Cell Surface Antigens, β-2 Microglobulins, Major Histocompatibility Antigens, Thy-1, Complement, C-Reactive Protein, Thymopoietin, Integrins, Post-gamma Globulin, α-2 Macroglobulins, and Other Related Proteins,” U.S. Dept. of Health and Human Services (1991). IgM constant regions can be numbered sequentially (i.e., amino acid #1 starting with the first amino acid of the constant region, or by using the Kabat numbering scheme. A comparison of the numbering of two alleles of the human IgM constant region sequentially (presented herein as SEQ ID NO: 1 (allele IGHM*03) and SEQ ID NO: 60 (allele IGHM*04)) and by the Kabat system is set out below. In each sequence provided herein which includes an IgM heavy chain constant region or multimerizing fragment thereof, any allele can be substituted for allele IGHM*03, which is presented, e.g., in SEQ ID NOs 2-4, and 11. The underlined amino acid residues are not accounted for in the Kabat system (“X,” double underlined below, can be serine (S) (SEQ ID NO: 1) or glycine (G) (SEQ ID NO: 60)):

| Sequential (SEQ ID NO: 1 or SEQ ID NO: 60)/KABAT numbering key | |
|---|---|
| for IgM heavy chain | |
| 1/127 | GSASAPTLFP LVSCENSPSD TSSVAVGCLA QDFLPDSITF SWKYKNNSDI |
| 51/176 | SSTRGFPSVL RGGKYAATSQ VLLPSKDVMQ GTDEHVVCKV QHPNGNKEKN |
| 101/226 | VPLPVIAELP PKVSVFVPPR DGFFGNPRKS KLICQATGFS PRQIQVSWLR |
| 151/274 | EGKQVGSGVT TDQVQAEAKE SGPTTYKVTS TLTIKESDWL XQSMFTCRVD |
| 201/324 | HRGLTFQQNA SSMCVPDQDT AIRVFAIPPS FASIFLTKST KLTCLVTDLT |
| 251/374 | TYDSVTISWT RQNGEAVKTH TNISESHPNA TFSAVGEASI CEDDWNSGER |
| 301/424 | FTCTVTHTDL PSPLKQTISR PKGVALHRPD VYLLPPAREQ LNLRESATIT |
| 351/474 | CLVTGFSPAD VFVQWMQRGQ PLSPEKYVTS APMPEPQAPG RYFAHSILTV |
| 401/524 | SEEEWNTGET YTCVVAHEAL PNRVTERTVD KSTGKPTLYN VSLVMSDTAG |
| 451/574 | TCY |

By “specifically binds,” it is generally meant that a binding molecule as provided herein binds to an epitope on a binding partner via a binding polypeptide, and that the binding entails some complementarity between the binding polypeptide and the binding partner. According to this definition, a binding molecule is said to “specifically bind” to a binding partner when it binds to that binding partner, via its binding polypeptide more readily than it would bind to a random, unrelated binding partner. The term “specificity” is used herein to qualify the relative affinity by which a certain binding molecule binds to a certain binding partner. For example, binding molecule “A” can be deemed to have a higher specificity for a given binding partner than binding molecule “B,” or binding molecule “A” can be said to bind to binding partner “C” with a higher specificity than it has for related binding partner “D.”

Binding molecules as provided herein can be derived from any animal origin including birds and mammals. The binding molecules can be human, murine, donkey, rabbit, goat, guinea pig, camel, llama, horse, or chicken binding molecules.

As used herein, the term “heavy chain subunit” includes amino acid sequences derived from an immunoglobulin heavy chain, a binding molecule as provided herein that includes a heavy chain subunit can include at least one of: a CH1 domain, a hinge (e.g., upper, middle, and/or lower hinge region) domain, a CH2 domain, a CH3 domain, a CH4 domain, a tailpiece, or a variant or fragment thereof provided that the resulting binding molecule can multimerize. For example, a binding molecule, or fragment, e.g., multimerizing fragment, variant, or derivative thereof can include without limitation a CH1 domain; a CH1 domain, a hinge, and a CH2 domain; a CH1 domain and a CH3 domain; a CH1 domain, a hinge, and a CH3 domain; or a CH1 domain, a hinge domain, a CH2 domain, and a CH3 domain. In certain embodiments a binding molecule or fragment, e.g., multimerizing fragment, variant, or derivative thereof can include a CH3 domain and a CH4 domain; or a CH3 domain, a CH4 domain, and a J-chain. Further, a binding molecule can lack certain constant region portions, e.g., all or part of a CH2 domain. It will be understood by one of ordinary skill in the art that these domains (e.g., the heavy chain subunit) can be modified such that they vary in amino acid sequence from the original immunoglobulin molecule. According to embodiments of the present disclosure, an IgM antibody, IgM-like antibody, or other IgM-derived binding molecule as provided herein comprises sufficient portions of an IgM heavy chain constant region to allow the IgM antibody, IgM-like antibody, or other IgM-derived binding molecule to form a multimer, e.g., a hexamer or a pentamer. As used herein such a fragment comprises a “multimerizing fragment.”

As used herein, the term “light chain subunit” includes amino acid sequences derived from an immunoglobulin light chain. The light chain subunit includes a CL (e.g., Cκ or Cλ) domain.

Binding molecules as provided herein can be described or specified in terms of the binding partner(s) that they recognize or specifically bind. A binding partner can include a single epitope or at least two epitopes, and can include any number of epitopes, depending on the size, conformation, and type of binding partner.

As used herein the term “disulfide bond” includes the covalent bond formed between two sulfur atoms, e.g., in cysteine residues of a polypeptide. The amino acid cysteine includes a thiol group that can form a disulfide bond or bridge with a second thiol group. Disulfide bonds can be “intra-chain,” i.e., linking to cysteine residues in a single polypeptide or polypeptide subunit, or can be “inter-chain,” i.e., linking two separate polypeptide subunits, e.g., an antibody heavy chain and an antibody light chain, two antibody heavy chains, or an IgM or IgA antibody heavy chain constant region and a J-chain.

The term “multispecific binding molecule, e.g., “bispecific binding molecule” refers to a binding molecule as provided herein that has binding polypeptides that bind to two or more different binding partners, or different epitopes of a single binding partner.

As used herein, the terms “linked,” “fused” or “fusion” or other grammatical equivalents can be used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence.

In the context of polypeptides, a “linear sequence” or a “sequence” is an order of amino acids in a polypeptide in an amino to carboxyl terminal direction in which amino acids that neighbor each other in the sequence are contiguous in the primary structure of the polypeptide. A portion of a polypeptide that is “amino-terminal” or “N-terminal” to another portion of a polypeptide is that portion that comes earlier in the sequential polypeptide chain. Similarly, a portion of a polypeptide that is “carboxy-terminal” or “C-terminal” to another portion of a polypeptide is that portion that comes later in the sequential polypeptide chain. For example, in a typical binding molecule as provided herein, the binding polypeptide is “N-terminal” to the immunoglobulin constant region, and the constant region is “C-terminal” to the binding polypeptide.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into RNA, e.g., messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the terms “signal transduction” or “cell signaling” refer to the transmission of molecular or biochemical signals from the outside of a cell to the interior of the cell, e.g., through binding of a ligand to a receptor expressed on the surface of a cell. The signal can be transmitted through one or more biochemical events in the cell, e.g., protein phosphorylation by various protein kinases, ultimately resulting in a cellular response such as, but not limited to, cellular activation (e.g., production of cytokines), cell proliferation, apoptosis, or morphogenesis. For example, when a ligand contacts the portion of a receptor exposed on the surface of a cell, a biochemical cascade is initiated through the intracellular portion of the receptor in the cell resulting in, e.g., transcription or translation of genes, or gene products, post-translational modifications or conformational changes in proteins, or translocation of proteins. See, e.g., Bradshaw, Ralph A.; Dennis, Edward A., eds. (2010). Handbook of Cell Signaling (2nd ed.). Amsterdam, Netherlands: Academic Press.

As used herein, “modulation” of signal transduction can include any intervention which affects normal signal transduction, e.g., enhances signal transduction, initiates signal transduction where signal transduction would normally be blocked, inhibits or retards signal transduction, or blocks signal transduction where signal transduction would normally be active. As used herein, an “agonist” of a signal transduction pathway enhances signal transduction or initiates signal transduction where signal transduction would normally be blocked, and an “antagonist” of signal transduction inhibits or blocks signal transduction. Signal transduction agonists typically act directly on a signal transduction pathway, e.g., by interacting with a receptor on the surface of a cell much as the native ligand would act. Antagonists of signal transduction can act directly on a signal transduction pathway, e.g., by blocking a receptor from binding to its native ligand, or can act indirectly, e.g., by binding to and thereby diverting a ligand from binding to its receptor (e.g., a “decoy receptor” or a “receptor ectodomain”) or by allosterically altering the ligand or receptor binding domain such that signal transduction can no longer occur.

Terms such as “treating” or “treatment” or “to treat” or “alleviating” or “to alleviate” refer to therapeutic measures that cure, slow down, lessen symptoms of, and/or halt or slow the progression of an existing diagnosed pathologic condition or disorder in a subject that has that disorder or pathologic condition. Terms such as “prevent,” “prevention,” “avoid,” “deterrence” and the like refer to prophylactic or preventative measures that prevent the development of an undiagnosed targeted pathologic condition or disorder. Thus, “a subject in need of treatment” can include those subjects already diagnosed with the disorder; those subjects prone to have the disorder; and those subjects in whom the disorder is to be prevented.

By “subject” or “individual” or “animal” or “patient” or “mammal,” is meant any subject, particularly a mammalian subject or a human subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

As used herein, phrases such as “a subject that would benefit from therapy” and “an animal in need of treatment” includes subjects, such as mammalian subjects, that would benefit from administration of a binding molecule as provided herein, that includes one or more antigen binding domains. Such binding molecules, e.g., antibodies, can be used, e.g., for diagnostic procedures and/or for treatment or prevention of a disease.

As used herein the terms “serum half-life” or “plasma half-life” refer to the time it takes (e.g., in minutes, hours, or days) following administration for the serum or plasma concentration of a drug, e.g., a multimeric binding molecule as provided herein, to be reduced by 50%. Two half-lives can be described: the alpha half-life, a half-life, or t_{1/2}α, which is the rate of decline in plasma concentrations due to the process of drug redistribution from the central compartment, e.g., the blood in the case of intravenous delivery, to a peripheral compartment (e.g., a tissue or organ), and the beta half-life, β half-life, or t_{1/2}β, which is the rate of decline due to the processes of excretion or metabolism.

As used herein the term “area under the plasma drug concentration-time curve” or “AUC” reflects the actual body exposure to drug after administration of a dose of the drug and is expressed in mg*h/L. This area under the curve is measured from time 0 (t0) to infinity (∞) and is dependent on the rate of elimination of the drug from the body and the dose administered. As used herein, the term “mean residence time” or “MRT” refers to the average length of time the drug remains in the body.

Multimeric Binding Molecules

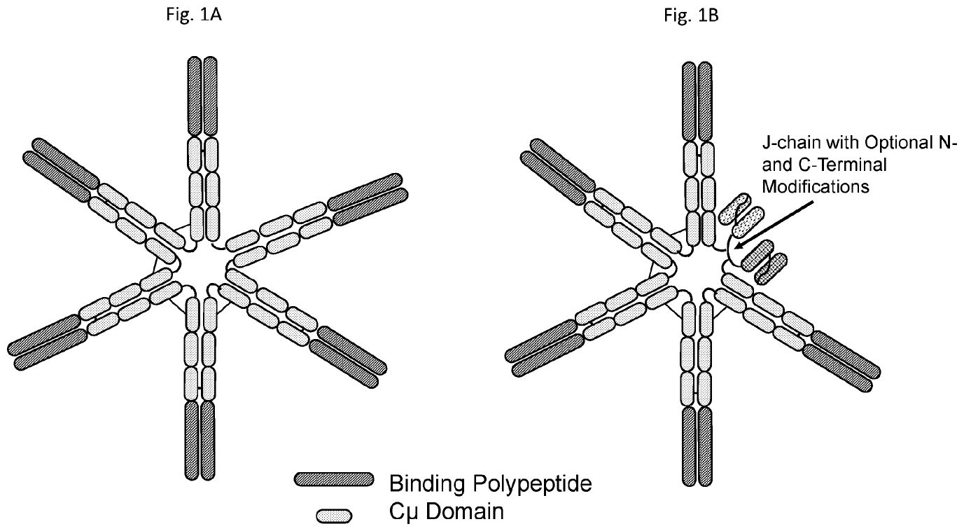

This disclosure provides a multimeric binding molecule that includes two or more, e.g., two, five, or six bivalent binding units or variants or fragments thereof, where each binding unit of the multimeric binding molecule includes two IgA or IgM heavy chain constant regions or multimerizing fragments or variants thereof, where at least three, at least four, at least five, at least six, at least seven, at least eight, at least nine, at least ten, at least eleven, at least twelve of the IgA or IgM constant regions or fragments thereof, or in certain embodiments each IgA or IgM heavy chain constant region or fragment thereof, is/are fused to a binding polypeptide or fragment thereof that specifically binds to a binding partner. Exemplary binding polypeptides and binding partners are described in detail elsewhere herein. In certain embodiments, a binding polypeptide or fragment thereof that is part of a binding molecule provided herein is not an antibody, an antigen-binding fragment of an antibody, or a variant or derivative of an antibody or antigen-binding fragment of an antibody. In certain embodiments, at least three, at least four, at least five, at least six, at least seven, at least eight, at least nine, at least ten, at least eleven or twelve of the binding polypeptides included in a binding molecule as provided herein bind to the same binding partner. In certain embodiments, at least three, at least four, at least five, at least six, at least seven, at least eight, at least nine, at least ten, at least eleven or twelve of the binding polypeptides included in a binding molecule as provided herein are identical. Structures of exemplary hexameric and pentameric IgM-derived binding molecules provided by this disclosure are diagrammed in FIG. 1A and FIG. 1B.

Multimeric binding molecules as provided herein can, in certain embodiments modulate signal transduction in a cell, e.g., a cell that typically expresses the binding polypeptide on its surface or a cell that typically expresses the binding partner on its surface. By “modulate signal transduction” is meant to affect signal transduction in a cell, e.g., initiate signal transduction in a cell where the signal transduction pathway is currently inactive, increase signal transduction activity in a pathway that is active but at lower levels, block or inhibit a signal transduction pathway, or reduce the activity level of an active signal transduction pathway. Modulation of signal transduction can in some instances be direct, e.g., where the binding molecule directly binds to a binding partner on the surface of a cell, thereby affecting signal transduction through that binding partner. Modulation of signal transduction can in some instances be indirect, e.g., where the binding molecule doesn't directly bind to the cell in which the signal transduction pathway is affected, but rather binds to a moiety that would otherwise bind to the cell as part of a signal transduction pathway. The binding molecule thereby can indirectly affect signal transduction by preventing that moiety from binding to the cell or reducing the concentration of that moiety available to bind to the cell. A multimeric binding molecule as provided herein that initiates or increases activity of a certain signal transduction pathway in a cell is an “agonist” of that pathway. A multimeric binding molecule as provided herein that reduces activity of a signal transduction pathway or blocks a signal transduction pathway is an “antagonist” of that signal transduction pathway. In certain embodiments, a multimeric binding molecule as provided herein can modulate signal transduction of a cell at a higher potency than an equivalent amount of a monomeric or dimeric binding molecule that includes one or two binding polypeptides binding to the same binding partner, e.g., one of two copies of the same binding polypeptide.

In certain embodiments, examples of which are provided herein, a binding partner is expressed on the surface of a cell, and binding of the binding polypeptide to the binding partner modulates signal transduction in that cell. For example, the binding polypeptide can be a ligand or a receptor-binding fragment of a ligand, and the binding partner can be a receptor expressed on the surface of the cell, where binding of the ligand and receptor can, for example, induce, increase inhibit, or block signal transduction through the receptor. In other embodiments, the binding polypeptide can be, e.g., a cytokine or receptor-binding fragment thereof, a growth factor or receptor binding fragment thereof, a neurotransmitter or receptor binding fragment thereof, a peptide or protein hormone or receptor binding fragment thereof, an immune checkpoint modulator ligand or receptor-binding fragment thereof, or a receptor-binding fragment of an extracellular matrix protein. In certain embodiments at least three, at least four, at least five, at least six, at least seven, at least eight, at least nine, at least ten, at least eleven or twelve of the binding polypeptides of the binding molecule bind to and modulate signal transduction through the same binding partner of the cell. In certain embodiments at least three, at least four, at least five, at least six, at least seven, at least eight, at least nine, at least ten, at least eleven or twelve of the binding polypeptides of the binding molecule are identical. In certain embodiments, contact of the binding molecule with three, four, five, six, seven, eight, nine, ten, eleven, or twelve copies of the binding partner on the cell can, e.g., induce, increase, inhibit, or block signal transduction in the cell at a higher potency than an equivalent amount of a monovalent or divalent binding molecule that has only one or two binding polypeptides binding to the same binding partner.

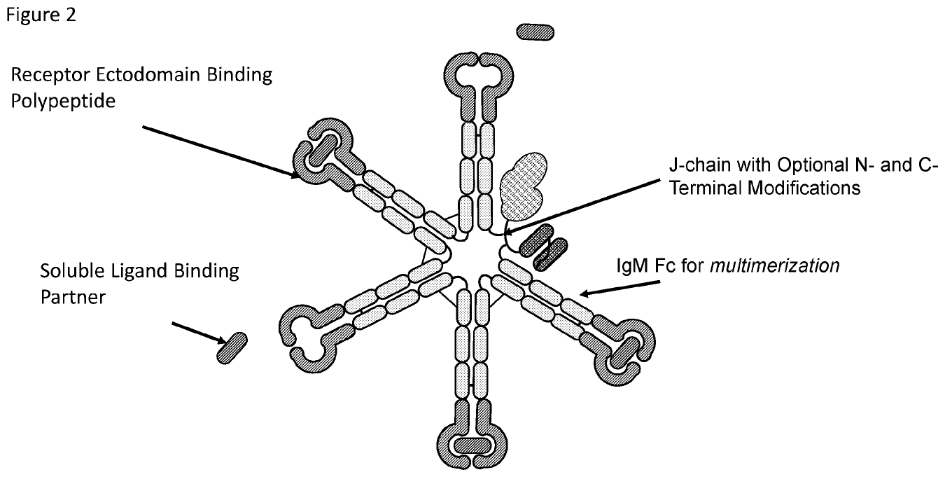

In certain embodiments, examples of which are provided herein, at least three, at least four, at least five, at least six, at least seven, at least eight, at least nine, at least ten, at least eleven or twelve of the binding polypeptides of the binding molecule include a receptor ectodomain that can specifically bind to a binding partner that includes a ligand or receptor-binding fragment thereof. As used herein, a “receptor ectodomain” refers to a portion of a receptor typically expressed on a cell which is exposed extracellularly. Accordingly, a “receptor ectodomain” would not include the transmembrane or intracellular portions of a receptor protein. According to these embodiments, the binding partner can be associated with a cell, e.g., expressed on the surface of a cell, or can be an extracellular moiety or a soluble fragment of a cell-associated moiety. In certain embodiments, the receptor ectodomain is not an antibody or antigen-binding fragment of an antibody. Also, according to these embodiments, binding of the receptor ectodomain to the ligand or fragment thereof can, typically indirectly, modulate signal transduction in a cell that expresses the receptor. For example, binding of the receptor ectodomains of the binding molecule to respective ligands or fragments thereof can competitively inhibit the ligands from associating with cell-expressed receptors, thereby inhibiting signal transduction in the cell. Competitive inhibition can be through, e.g., increased affinity for ligand binding, through an increased quantity of receptor ectodomains relative to the number of cell-expressed receptors, or a combination thereof. In certain embodiments, at least three, at least four, at least five, at least six, at least seven, at least eight, at least nine, at least ten, at least eleven or twelve of the receptor ectodomains bind to the same ligand. In certain embodiments, at least three, at least four, at least five, at least six, at least seven, at least eight, at least nine, at least ten, at least eleven or twelve of the receptor ectodomains are identical. In certain embodiments, contact of the receptor ectodomains of the binding molecule with its respective ligands or fragments thereof can, e.g., inhibit, or block signal transduction in a cell that expresses the receptor at a higher potency than an equivalent amount of a monovalent or divalent binding molecule that has only one or two receptor ectodomains binding to the same ligand. A schematic of a multimeric binding molecule where the binding polypeptides are receptor ectodomains is presented as FIG. 2.

IgM-Derived Multimeric Binding Molecules

In certain embodiments, the multimeric binding molecule provided by this disclosure is a hexameric or pentameric binding molecule that includes IgM heavy chain constant regions, or multimerizing fragments thereof fused to binding polypeptides as described herein. As provided herein, an IgM-derived binding molecule includes at least three, at least four, at least five, at least six, at least seven, at least eight, at least nine, at least ten, at least eleven, or twelve binding polypeptides that specifically bind to a binding partner, fused N-terminal to the IgM heavy chain constant regions or multimerizing fragments thereof of the multimeric binding molecule. In certain embodiments, at least three, at least four, at least five, at least six, at least seven, at least eight, at least nine, at least ten, at least eleven, or twelve binding polypeptides of the multimeric binding molecule bind to the same binding partner. In certain embodiments, at least three, at least four, at least five, at least six, at least seven, at least eight, at least nine, at least ten, at least eleven, or twelve binding polypeptides of the multimeric binding molecule are identical.

A bivalent IgM-derived binding unit as provided herein includes two IgM heavy chain constant regions, and an IgM-derived binding molecule typically includes five or six binding units. A full-length IgM heavy (μ) chain constant region includes four constant region domains, Cμ1 (also referred to as CM1, CMu1, or CH1), Cμ2 (also referred to as CM2, CMu2, or CH2), Cμ3 (also referred to as CM3, CMu3, or CH3), and Cμ4 (also referred to as CM4, CMu4, or CH4), and a “tailpiece” (tp). The human IgM constant region typically includes the amino acid sequence SEQ ID NO: 1 (identical to, e.g., GenBank Accession Nos. pir∥S37768, CAA47708.1, and CAA47714.1, allele IGHM*03) or SEQ ID NO: 60 (identical to, e.g., GenBank Accession No. sp|P01871.4, allele IGHM*04). The human Cμ1 domain extends from about amino acid 5 to about amino acid 102 of SEQ ID NO: 1 or SEQ ID NO: 60; the human Cμ2 domain extends from about amino acid 114 to about amino acid 205 of SEQ ID NO: 1 or SEQ ID NO: 60, the human Cμ3 domain extends from about amino acid 224 to about amino acid 319 of SEQ ID NO: 1 or SEQ ID NO: 60, the Cu 4 domain extends from about amino acid 329 to about amino acid 430 of SEQ ID NO: 1 or SEQ ID NO: 60, and the tailpiece (tp) extends from about amino acid 431 to about amino acid 453 of SEQ ID NO: 1 or SEQ ID NO: 60.

Five IgM-derived binding units can form a complex with an additional small polypeptide chain (the J-chain) to form an IgM binding molecule. The precursor human J-chain includes the amino acid sequence SEQ ID NO: 14.

The mature human J-chain includes the amino acid sequence SEQ ID NO: 15. Without the J-chain, IgM-derived binding units typically assemble into a hexamer. While not wishing to be bound by theory, the assembly of IgM binding units into a pentameric or hexameric binding molecule is thought to involve at least the Cμ4, and/or tp domains. See, e.g., Braathen, R., et al., J. Biol. Chem. 277:42755-42762 (2002). Accordingly, a pentameric or hexameric binding molecule provided in this disclosure typically includes IgM constant regions that include at least the Cμ4, and/or tp domains.

An IgM heavy chain constant region can additionally include a Cμ3 domain or a fragment thereof, a Cμ2 domain or a fragment thereof, a Cμ1 domain or a fragment thereof, and/or other IgM or other immunoglobulin heavy chain domains. In certain embodiments, a binding molecule as provided herein can include a complete IgM heavy (μ) chain constant region, e.g., SEQ ID NO: 1 or SEQ ID NO: 60, or a variant, derivative, or analog thereof.

In certain embodiments each binding unit of a multimeric binding molecule as provided herein includes two IgM heavy chain constant regions or multimerizing fragments or variants thereof, each including at least an IgM Cμ4 domain and an IgM tailpiece domain. In certain embodiments the IgM heavy chain constant regions can each further include an IgM Cμ3 domain situated N-terminal to the IgM Cμ4 and IgM tailpiece domains.

In certain embodiments the IgM heavy chain constant regions can each further include an IgM Cμ2 domain situated N-terminal to the IgM Cμ3 domain. Exemplary multimeric binding molecules provided herein include human IgM constant regions that include SEQ ID NO: 3 which includes the wild-type human Cμ2, Cμ3, Cμ4-TP domains.

In certain IgM-derived multimeric binding molecules as provided herein each IgM constant region can include, instead of, or in addition to an IgM Cμ2 domain, an IgG hinge region or functional variant thereof situated N-terminal to the IgM Cμ3 domain. An exemplary variant human IgG1 hinge region amino acid sequence in which the cysteine at position 6 is substituted with serine is VEPKSSDKTHTCPPCPAP (SEQ ID NO: 5). An exemplary IgM constant region of this type includes the variant human IgG1 hinge region fused to a multimerizing fragment of the human IgM constant region including the Cμ3, Cμ4, and TP domains, and includes the amino acid sequence SEQ ID NO: 6.

Modified Human IgM Constant Regions with Reduced CDC Activity, Altered Glycosylation, or Increased Serum Half-Life

In certain embodiments, a modified human IgM constant region, when expressed as part of a modified human IgM-derived binding molecule as provided herein exhibits reduced complement-dependent cytotoxicity (CDC) activity to cells in the presence of complement, relative to a corresponding wild-type human IgM constant region. By “corresponding wild-type human IgM constant region” is meant a wild-type IgM constant region that is identical to a modified IgM constant region except for the modification or modifications in the constant region affecting CDC activity. For example, the “corresponding wild-type human IgM constant region” will be fused to identical binding polypeptides and any other modifications or truncations that that the modified human IgM constant might have other than the modifications affecting CDC activity. In certain embodiments, the modified human IgM constant region includes one or more amino acid substitutions, e.g., in the Cμ3 domain, relative to a wild-type human IgM constant region as described, e.g., in PCT Publication No. WO 2018/187702, which is incorporated herein by reference in its entirety. Assays for measuring CDC are well known to those of ordinary skill in the art, and exemplary assays are described e.g., in PCT Publication No. WO 2018/187702.

In certain embodiments, the modified human IgM constant region as provided herein includes a substitution relative to a wild-type human IgM constant region at position P311 of SEQ ID NO: 1 OR SEQ ID NO: 60. In other embodiments the modified IgM constant region as provided herein contains a substitution relative to a wild-type human IgM constant region at position P313 of SEQ ID NO: 1 OR SEQ ID NO: 60. In other embodiments the modified IgM constant region as provided herein contains a combination of substitutions relative to a wild-type human IgM constant region at positions P311 of SEQ ID NO: 1 OR SEQ ID NO: 60 and P313 of SEQ ID NO: 1 OR SEQ ID NO: 60. The modified IgM constant region at amino acid position P311 of SEQ ID NO: 1 OR SEQ ID NO: 60 can be substituted with alanine (P311A), serine (P311S), or glycine (P311G). The modified IgM constant region at amino acid position P313 of SEQ ID NO: 1 OR SEQ ID NO: 60 can be substituted with alanine (P313A), serine (P313S), or glycine (P313G). The modified IgM constant region at amino acid positions P311 and P313 of SEQ ID NO: 1 OR SEQ ID NO: 60 can be substituted with alanine (P311A) and serine (P313S), respectively (SEQ ID NO: 2 or any combination of alanine, serine, and/or glycine.

In one embodiment, a binding molecule as provided herein including a modified human IgM constant region including an amino acid substitution at P311 and/or P313, e.g., P311A, P311S, P311G, P313A, P313S, and/or P313G or any combination thereof, has a maximum CDC achieved in a dose-response assay decreased by at least 10%, 20%, 30%, 40%, 50%, 60%, 70%, 80% or 90% relative to a binding molecule that includes a corresponding wild-type IgM constant region.

This disclosure therefore provides multimeric IgM-derived binding molecules where at least one binding unit includes, two or more binding units include, or each binding unit includes two modified IgM heavy chain constant regions or multimerizing fragments or variants thereof, which exhibit reduced CDC activity. In certain embodiments the modified IgM constant regions include an IgM Cμ3 domain and an IgM tailpiece domain and further include a modified IgM Cμ3 domain situated N-terminal to the IgM Cμ3 and IgM tailpiece domains. In certain embodiments the IgM heavy chain constant regions can each further include an IgM Cμ2 domain situated N-terminal to the modified IgM Cμ3 domain. Exemplary multimeric binding molecules provided herein include human IgM constant regions that include SEQ ID NO: 4 which includes a human Cμ2 domain, a modified human Cμ3 domain that includes P311A and P313S mutations, and human Cμ4-TP domains. In certain embodiments, a multimeric binding molecule in which the IgM heavy chain constant regions include the amino acid sequence SEQ ID NO: 4 has reduced CDC activity relative to a corresponding binding molecule in which the IgM heavy chain constant regions include the amino acid sequence SEQ ID NO: 3.

In certain IgM-derived multimeric binding molecules with reduced CDC activity as provided herein each IgM constant region can include, instead of, or in addition to an IgM domain, an IgG hinge region or functional variant thereof situated N-terminal to the variant IgM Cμ3 domain. An exemplary variant human IgG1 hinge region amino acid sequence is VEPKSSDKTHTCPPCPAP (SEQ ID NO: 5). Exemplary multimeric binding molecules provided herein include human IgM constant regions that include SEQ ID NO: 7 which includes a modified human IgG1 hinge region, a modified human Cμ3 domain that includes P311A and P313S mutations, and human Cμ4-TP domains. In certain embodiments, a multimeric binding molecule in which the IgM heavy chain constant regions include the amino acid sequence SEQ ID NO: 7 has reduced CDC activity relative to a corresponding binding molecule in which the IgM heavy chain constant regions that include the amino acid sequence SEQ ID NO: 6.

Certain IgM-derived binding molecules as provided herein can be engineered to have enhanced serum half-life. Exemplary IgM heavy chain constant region mutations that can enhance serum half-life of an IgM-derived binding molecule are disclosed in PCT Publication No. WO 2019/169314, the contents of which is incorporated by reference herein in its entirety. For example, in addition to one or more of the glycosylation mutations described elsewhere herein, a variant IgM heavy chain constant region of an IgM-derived binding molecule as provided herein can include an amino acid substitution at an amino acid position corresponding to amino acid 5401, E402, E403, R344, and/or E345 of a wild-type human IgM constant region (e.g., SEQ ID NO: 1 or SEQ ID NO: 60). By “an amino acid corresponding to amino acid 5401, E402, E403, R344, and/or E345 of a wild-type human IgM constant region” is meant the amino acid in the sequence of the IgM constant region of any species which is homologous to 5401, E402, E403, R344, and/or E345 in the human IgM constant region. In certain embodiments, the amino acid corresponding to 5401, E402, E403, R344, and/or E345 of SEQ ID NO: 1 or SEQ ID NO: 60 can be substituted with any amino acid, e.g., alanine.

Human IgM constant regions, and also certain non-human primate IgM constant regions, as provided herein typically include five (5) naturally-occurring asparagine (N)-linked glycosylation motifs or sites. As used herein “an N-linked glycosylation motif” comprises or consists of the amino acid sequence N-X1-S/T, wherein N is asparagine, X1 is any amino acid except proline (P), and S/T is serine (S) or threonine (T). The glycan is attached to the nitrogen atom of the asparagine residue. See, e.g., Drickamer K, Taylor M E (2006), Introduction to Glycobiology (2nd ed.). Oxford University Press, USA. N-linked glycosylation motifs occur in the human IgM heavy chain constant regions of SEQ ID NO: 1 or SEQ ID NO: 60 starting at positions 46 (“N1”), 209 (“N2”), 272 (“N3”), 279 (“N4”), and 440 (“N5”). These five motifs are conserved in non-human primate IgM heavy chain constant regions, and four of the five are conserved in the mouse IgM heavy chain constant region. Each of these sites in the human IgM heavy chain constant region, except for N4, can be mutated to prevent glycosylation at that site, while still allowing IgM expression and assembly into a hexamer or pentamer. See U.S. Provisional Application No. 62/891,263, filed on Aug. 23. 2019, the contents of which is incorporated herein by reference in its entirety.

IgA-Derived Binding Molecules

In certain embodiments, the multimeric binding molecule provided by this disclosure is a dimeric binding molecule that includes IgA heavy chain constant regions, or multimerizing fragments thereof. As provided herein, an IgA-derived binding molecule includes at least three or all four binding polypeptides that specifically bind to a binding partner, fused N-terminal to the IgA heavy chain constant regions or multimerizing fragments thereof of the multimeric binding molecule. In certain embodiments, at least three or all four binding polypeptides of the multimeric binding molecule bind to the same binding partner. In certain embodiments, at least three or all four binding polypeptides of the multimeric binding molecule are identical.

A bivalent IgA-derived binding unit includes two IgA heavy chain constant regions, and a dimeric IgA-derived binding molecule includes two binding units. IgA contains the following heavy chain constant domains, Cα1 (or alternatively CA1 or CH1), a hinge region, Cα2 (or alternatively CA2 or CH2), and Cα3 (or alternatively CA3 or CH3), and a C-terminal “tailpiece.” Human IgA has two subtypes, IgA1 and IgA2. The human IgA1 constant region typically includes the amino acid sequence SEQ ID NO: 24 The human Cal domain extends from about amino acid 6 to about amino acid 98 of SEQ ID NO: 24; the human IgA1 hinge region extends from about amino acid 102 to about amino acid 124 of SEQ ID NO: 24, the human Cα2 domain extends from about amino acid 125 to about amino acid 219 of SEQ ID NO: 24, the human Cα3 domain extends from about amino acid 228 to about amino acid 330 of SEQ ID NO: 24, and the tailpiece extends from about amino acid 331 to about amino acid 352 of SEQ ID NO: 24. The human IgA2 constant region typically includes the amino acid sequence SEQ ID NO: 25. The human Cal domain extends from about amino acid 6 to about amino acid 98 of SEQ ID NO: 25; the human IgA2 hinge region extends from about amino acid 102 to about amino acid 111 of SEQ ID NO: 25, the human Cα2 domain extends from about amino acid 113 to about amino acid 206 of SEQ ID NO: 25, the human Cα3 domain extends from about amino acid 215 to about amino acid 317 of SEQ ID NO: 25, and the tailpiece extends from about amino acid 318 to about amino acid 340 of SEQ ID NO: 25.

Two IgA binding units can form a complex with two additional polypeptide chains, the J chain (SEQ ID NO: 15) and the secretory component (precursor, SEQ ID NO: 26, mature, SEQ ID NO: 27) to form a bivalent secretory IgA (sIgA)-derived binding molecule as provided herein. While not wishing to be bound by theory, the assembly of two IgA binding units into a dimeric IgA-derived binding molecule is thought to involve the Cα3 and tailpiece domains. See, e.g., Braathen, R., et al., J. Biol. Chem. 277:42755-42762 (2002). Accordingly, a multimerizing dimeric IgA-derived binding molecule provided in this disclosure typically includes IgA constant regions that include at least the Cα3 and tailpiece domains.

An IgA heavy chain constant region can additionally include a Cα2 domain or a fragment thereof, an IgA hinge region or fragment thereof, a Cα1 domain or a fragment thereof, and/or other IgA (or other immunoglobulin, e.g., IgG) heavy chain domains, including, e.g., an IgG hinge region. In certain embodiments, a binding molecule as provided herein can include a complete IgA heavy (α) chain constant domain (e.g., SEQ ID NO: 24 or SEQ ID NO: 25), or a variant, derivative, or analog thereof.

In certain embodiments each binding unit of a multimeric binding molecule as provided herein includes two IgA heavy chain constant regions or multimerizing fragments or variants thereof, each including at least an IgA Cα3 domain and an IgA tailpiece domain. In certain embodiments the IgA heavy chain constant regions can each further include an IgA Cα2 domain situated N-terminal to the IgA Cα3 and IgA tailpiece domains. For example, the IgA heavy chain constant regions can include amino acids 125 to 353 of SEQ ID NO: 24 or amino acids 113 to 340 of SEQ ID NO: 25. In certain embodiments the IgA heavy chain constant regions can each further include an IgA or IgG hinge region situated N-terminal to the IgA Cα2 domains. For example, the IgA heavy chain constant regions can include amino acids 102 to 353 of SEQ ID NO: 24 or amino acids 102 to 340 of SEQ ID NO: 25. In certain embodiments the IgA heavy chain constant regions can each further include an IgA Cα1 domain situated N-terminal to the IgA hinge region.

Binding Polypeptides and Binding Partners

A multimeric binding molecule as provided herein can include a large variety of non-limiting binding polypeptides. For example, where the binding partner is expressed on the surface of a cell, the binding polypeptide can be, for example, a ligand or receptor-binding fragment of a ligand (e.g., where the ligand is itself typically expressed on the surface of another cell), a cytokine or receptor-binding fragment thereof, a growth factor or receptor binding fragment thereof, a neurotransmitter or receptor binding fragment thereof, a peptide or protein hormone or receptor binding fragment thereof, an immune checkpoint modulator ligand or receptor-binding fragment thereof, or a receptor-binding fragment of an extracellular matrix protein. A binding molecule as provided herein need only include those portions of a binding polypeptide required to bind to the binding partner, and either directly or indirectly modulate signal transduction in a cell.

By “ligand” is broadly meant signaling molecules that can bind to cell-surface receptors, thereby causing change, e.g., a conformational change, in the receptor thereby triggering an event in the cell expressing the receptor. Hundreds, if not thousands of individual ligands have been identified and characterized and are typically organized by families either by structure or function. Ligand families include but are not limited to: activin and inhibin ligands, bone morphogenetic proteins, chemokines, complement components, ephrins, fibroblast growth factor (FGF) family ligands, galectins, glycoprotein hormones, immune checkpoint modulators, interferons, interleukins, neuropeptides, tumor necrosis factor superfamily (TNFSF) ligands, vascular endothelial growth factor (VEGF) family ligands, TNF-β superfamily ligands, and wnt family ligands. See, e.g., IUPHAR/BPS Guide to PHARMACOLOGY (www_dot_guidetopharmacology_dot_org/GRAC/LigandFamiliesForward, and www_dot_guidetopharmacology_dot_org/GRAC/LigandListForward, both sites last visited on Jul. 23, 2018). Ligands can be soluble molecules in the extracellular milieu or can themselves be expressed on the surface of a cell.

In certain embodiments, a binding polypeptide of a multimeric binding molecule as provided herein can be a tumor necrosis factor superfamily (TNFSF) ligand. These ligands bind to and activate receptors of the TNF receptor superfamily (TNFrSF), triggering a large variety of functions in receptor-expressing cells, e.g., inflammation, apoptosis, cell proliferation, cell invasion, angiogenesis, or cell differentiation. See, e.g., Aggarwal, B. B. et al., Blood 119:651-665 (2012). The TNF superfamily includes at least 19 ligands and 29 interacting receptors including, but not limited to, TNF-α (also known as cachectin, exemplary human sequence presented as SEQ ID NO: 28, ectodomain: amino acids 57-233 of SEQ ID NO: 28), TNF-β (also known as lymphotoxin-alpha, exemplary human sequence presented as SEQ ID NO: 29, mature soluble protein: amino acids 35-205 of SEQ ID NO: 29), lymphotoxin-β (LT-β) (exemplary human sequence presented as SEQ ID NO: 30, ectodomain: amino acids 49-244 of SEQ ID NO: 30), OX40L (also known as gp34 or CD252, exemplary human sequence presented as SEQ ID NO: 31, ectodomain: amino acids 51-183 of SEQ ID NO: 31), CD40L (exemplary human sequence presented as SEQ ID NO: 32, ectodomain: amino acids 47-261 of SEQ ID NO: 32), FasL (also known as apoptosis antigen ligand or APTL, exemplary human sequence presented as SEQ ID NO: 33, ectodomain: 103-281 of SEQ ID NO: 33), 4-1BBL (exemplary human sequence presented as SEQ ID NO: 34, ectodomain: amino acids 50-254 of SEQ ID NO: 34), TNF-related apoptosis-inducing ligand (TRAIL) (exemplary human sequence presented as SEQ ID NO: 35, ectodomain: amino acids 39-281 of SEQ ID NO: 35), and glucocorticoid-induced TNF receptor ligand (GITRL) (exemplary human sequence presented as SEQ ID NO: 36, ectodomain: amino acids 72-199 of SEQ ID NO: 36). Those of ordinary skill in the art will appreciate that various related human isoforms of these presented sequences exist, and orthologs exist in other species. Moreover, these ligands appear in the literature by many different names and acronyms but can be distinguished by their primary structures and functions. Many of the signal transduction pathways activated by these ligands are of therapeutic importance in treating, e.g., cancer, infectious diseases, inflammatory diseases, and/or neurodegenerative diseases.

A common feature of TNFSF-TNFrSF interactions is the requirement for the ligand to engage at least three receptor monomers on the cell surface in order for signal transduction to occur. TNFSF ligands, which typically assemble as homotrimers, are adapted to accomplish this task. See, e.g., Locksley, R. M., et al., Cell 104:487-501 (2001). As such, the binding molecules as provided herein, which can include three, four, five, six, seven, eight, nine, ten, eleven, or twelve TNFSF ligand binding polypeptides that can engage with a TNFrSF binding partner can act as superagonists for receptor activation. For example, a hexameric binding molecule as provided herein including up to twelve copies of a receptor-binding fragment of TRAIL, e.g., amino acids 39-281 of SEQ ID NO: 35, could, upon association with tumor cells over-expressing the death-domain containing receptors DR4 and/or DR5, highly efficiently induce apoptosis of those tumor cells.

In certain embodiments, a binding polypeptide of a multimeric binding molecule as provided herein can be an immune checkpoint modulator ligand or a receptor-binding fragment thereof. Immune checkpoint modulator ligands include, but are not limited to, programmed cell death 1 ligand 1 (PD-L1, also referred to as CD274, B7 homolog 1 or B7-H1, exemplary human sequence presented as SEQ ID NO: 8, ectodomain: amino acids 19-238 of SEQ ID NO: 8, or SEQ ID NO: 9), CD80 (also referred to as B7-1, exemplary human sequence presented as SEQ ID NO: 37, ectodomain: amino acids 35-242 of SEQ ID NO: 37), and CD86 (also referred to as B7-2, exemplary human sequence presented as SEQ ID NO: 38). In certain embodiments, the binding partner is, e.g., a receptor in an immune checkpoint modulator pathway. For example, the binding partner can be PD-1 or CTLA4.

PD-L1 is a 40 kDa transmembrane protein typically expressed on a variety cells, including dendritic cells and monocytes. PD-L1 is the ligand of Programmed cell death protein-1 (PD-1). Binding of PD-L1 to the PD-1 receptor on e.g., activated T cells reduces proliferation of antigen-specific T cells, and can also reduce apoptosis of regulatory T cells (Tregs). Tumor cells can over-express PD-L1 leading to suppression of anti-tumor immunity (see, e.g., Dong H., et al., Nat. Med. 8:793-800 (2002)), but reduced expression of PD-L1 also associated with autoimmunity (see, e.g., Ansari, M. J., et al., J. Exp. Med. 198:63-69 (2003); Mozaffarian, N., et al., Rheumatology 47:1335-1341 (2008)). An exemplary amino acid sequence of the precursor human PD-L1 is presented as SEQ ID NO: 8.

The signal peptide of human PD-L1 extends from amino acid 1 to about amino acid 18 of SEQ ID NO: 8. The mature human PD-L1 protein extends from about amino acid 19 to amino acid 290 of SEQ ID NO: 8. Human PD-L1 has two extracellular domains, the Ig-like V-type domain that extends from about amino acid 19 to about amino acid 127 of SEQ ID NO: 8 and the Ig-like C2-type domain that extends from about amino acid 133 to about amino acid 225 of SEQ ID NO: 8. The transmembrane domain of human PD-L1 extends from about amino acid 239 to about amino acid 259 of SEQ ID NO: 8. The cytoplasmic domain of human PD-L1 extends from about 260 to amino acid 290 of SEQ ID NO: 8. As a transmembrane protein, it will be appreciated by those of ordinary skill in the art that a receptor-binding soluble fragment of PD-L1 would typically be included in a multivalent binding molecule as provided by this disclosure. Those of ordinary skill in the art will also appreciate that different receptor-binding isoforms and/or splice variants of human PD-L1 exist and can be included in a binding molecule as provided herein. Moreover, orthologs of human PD-L1 are present in other species, and receptor-binding fragments of PD-L1 of any species can be included in a multivalent binding molecule as provided herein.

In certain embodiments, the binding polypeptide of the multimeric binding molecule provided herein includes a receptor-binding fragment of PD-L1, e.g., human PD-L1. In certain embodiments the binding polypeptide includes the V-type ectodomain of PD-L1, e.g., human PD-L1, e.g., amino acids 18 to 127 or 19 to 127 of SEQ ID NO: 8. In certain embodiments the binding polypeptide includes amino acids 18 to 134 or 19 to 134 of SEQ ID NO: 8. See, e.g., Zak et al. Structure 23:2341-2348 (2015). In certain embodiments the binding polypeptide includes the V-type and C2-type ectodomains of PD-L1, e.g., human PD-L1, e.g., amino acids 18 to 238 or 19 to 238 of SEQ ID NO: 8 (an exemplary ectodomain of human PD-L1 is presented herein as SEQ ID NO: 9).

In certain embodiments, a binding polypeptide of a multimeric binding molecule as provided herein can be a receptor ectodomain. Examples include, but are not limited to, an ectodomain of a TNF superfamily receptor, an ectodomain of an immune checkpoint modulator receptor, an ectodomain of a TGF-β receptor, an ectodomain of a vascular endothelial growth factor receptor (VEGFR), or any combination thereof.

For example, the binding polypeptide can include a soluble ligand-binding fragment of a TNF superfamily receptor (TNFrSF), e.g., a soluble fragment of death domain containing receptor-4 (DR4, also known as TRAIL-R1 or APO2, exemplary human sequence presented as SEQ ID NO: 39, ectodomain: amino acids 24-239 of SEQ ID NO: 39); death domain containing receptor-5 (DR5, also known as TRAIL-R2, Ly98, or CD262, exemplary human sequence presented as SEQ ID NO: 40, ectodomain: amino acids 56-210 of SEQ ID NO: 40); OX-40 (exemplary human sequence presented as SEQ ID NO: 41, ectodomain: amino acids 29-214 of SEQ ID NO: 41); CD40 (exemplary human sequence presented as SEQ ID NO: 42, ectodomain: amino acids 21-193 of SEQ ID NO: 42); 4-1BB (also known as CD137, exemplary human sequence presented as SEQ ID NO: 43, ectodomain: amino acids 24-186 of SEQ ID NO: 43); and/or glucocorticoid-induced tumor necrosis factor receptor (GITR, also known as AITR or CD357, exemplary human sequence presented as SEQ ID NO: 44, ectodomain: amino acids 26-162 of SEQ ID NO: 44); or any combination thereof.

The binding polypeptide can also be, for example, an ectodomain of an immune checkpoint modulator receptor, e.g., a soluble ligand-binding fragment of cytotoxic T-lymphocyte-associated protein-4 (CTLA4, also known as CD152, exemplary human sequence presented as SEQ ID NO: 45, ectodomain: amino acids 36-161 of SEQ ID NO: 45), PD-1 (exemplary human sequence presented as SEQ ID NO: 46, ectodomain: amino acids 21-170 of SEQ ID NO: 46), LAG3 (also known as CD223, exemplary human sequence presented as SEQ ID NO: 47, ectodomain: amino acids 29-450 of SEQ ID NO: 47); CD28 (exemplary human sequence presented as SEQ ID NO: 48, ectodomain: amino acids 19-152 of SEQ ID NO: 48); immunoglobulin-like domain containing receptor 2 (ILDR2, exemplary human sequence presented as SEQ ID NO: 49, ectodomain: amino acids 21-186 of SEQ ID NO: 49), T-cell immunoglobulin mucin family member 3 (TIM-3, also known as CD366, exemplary human sequence presented as SEQ ID NO: 50, ectodomain: amino acids 22-202 of SEQ ID NO: 50), or any combination thereof.

The binding polypeptide can also be, for example, an ectodomain of a transforming growth factor beta receptor (TGFβR). Three human receptor proteins are known to engage with TGFβ. These include TGFβ receptor type 1 (TGFβR1, also known as Activin receptor-like kinase 5 or ALKS, exemplary human sequence presented as SEQ ID NO: 51, ectodomain: amino acids 34-126 of SEQ ID NO: 51), TGFβ receptor type 2 (TGFβR2, exemplary human sequence presented as SEQ ID NO: 52, ectodomain: amino acids 23-166 of SEQ ID NO: 52), and TGFβ receptor type 3 (TGFβR3, exemplary human sequence presented as SEQ ID NO: 53, ectodomain: amino acids 21-787 of SEQ ID NO: 53).

The binding polypeptide can also be, for example, an ectodomain of a vascular endothelial growth factor receptor (VEGFR). Human receptors known to engage members of the vascular endothelial growth factor superfamily include, but are not limited to, vascular endothelial growth factor receptor 2 (VEGFR-2, exemplary human sequence presented as SEQ ID NO: 54, ectodomain, amino acids 20-764 of SEQ ID NO: 54), vascular endothelial growth factor receptor 3 (VEGFR-3, also known as FLT4, exemplary human sequence presented as SEQ ID NO: 55, ectodomain, amino acids 25-775 of SEQ ID NO: 55), and vascular endothelial growth factor receptor 1 (VEGFR-1, also known as FLT1, exemplary human sequence presented as SEQ ID NO: 56, ectodomain, amino acids 27-758 of SEQ ID NO: 56). VEGFR-2-ectodomain-Fcγ proteins have been shown to inhibit neovascularization in a mouse model of intracranial human glioblastoma multiforme. See, e.g., Szentirmai, O., et al., J. Neurosurg 108:979-988 (2008). An Fcγ fusion protein including ligand-binding ectodomains of VEGF receptors (aflibercept, SEQ ID NO: 57, contains amino acids 129-230 of SEQ ID NO: 56 (VEGFR-1), amino acids 225-327 of SEQ ID NO: 54 (VEGFR-2) and the human IgG1Fc constant region) is used for preventing ocular neovascularization associated, e.g., with age-related macular degeneration. See, e.g., Sawar, S., et al., Dev. Opthalmol. 55:282-294 (2016).

Modified J-Chains

In certain embodiments, the J-chain of a pentameric IgM-derived binding molecule as provided herein can be modified, e.g., by introduction of a heterologous moiety, or two or more heterologous moieties, without interfering with the ability of the pentameric IgM-derived binding molecule to assemble and bind to its binding partner(s). See, e.g., U.S. Pat. Nos. 9,951,134 and 10,400,038, in U.S. Patent Application Publication Nos. US-2019-0185570 and US-2018-0265596, each of which is incorporated herein by reference in its entirety. Accordingly, a pentameric IgM-derived binding molecule as provided herein can include a modified J-chain or functional fragment thereof that includes a heterologous moiety, or two or more heterologous moieties, introduced into the J-chain or fragment thereof. In certain embodiments a heterologous moiety incorporated into a modified J-chain can be a peptide or polypeptide sequence fused in frame to the J-chain or chemically conjugated to the J-chain. In certain embodiments a heterologous moiety incorporated into a modified J-chain can be a chemical moiety conjugated to the J-chain. Heterologous moieties to be attached to a J-chain can include, without limitation, a binding moiety, e.g., an antibody or antigen binding fragment thereof, e.g., a single chain Fv (scFv) molecule, a stabilizing peptide that can increase the half-life of the pentameric IgM-derived binding molecule, or a chemical moiety such as a polymer or a cytotoxin.

In some embodiments, a modified J-chain can include an antigen binding domain that can include without limitation a polypeptide (including small peptides) capable of specifically binding to a target antigen. In certain embodiments, an antigen binding domain associated with a modified J-chain can be an antibody or an antigen-binding fragment thereof, as described elsewhere herein. In certain embodiments the antigen binding domain can be a scFv binding domain or a single-chain binding domain derived, e.g., from a camelid or condricthoid antibody. The antigen binding domain can be introduced into the J-chain at any location that allows the binding of the antigen binding domain to its binding partner without interfering with J-chain function or the function of an associated IgM or IgA binding molecule. Insertion locations include but are not limited to: at or near the C-terminus, at or near the N-terminus or at an internal location that, based on the three-dimensional structure of the J-chain, is accessible. In certain embodiments, the antigen binding domain can be introduced into the mature human J-chain of SEQ ID NO: 15 between cysteine residues 92 and 101 of SEQ ID NO: 15. In a further embodiment, the antigen binding domain can be introduced into the human J-chain of SEQ ID NO: 15 at or near a glycosylation site. In a further embodiment, the antigen binding domain can be introduced into the human J-chain of SEQ ID NO: 15 within about 10 amino acid residues from the N-terminus or the C-terminus.

Pentameric IgM-Derived Binding Molecules with J-Chain Mutations that Alter Serum Half-Life

This disclosure provides an IgM-derived pentameric binding molecule that includes IgM heavy chain constant regions or multimerizing fragments thereof, where the binding molecule has enhanced serum half-life relative to that typically observed for IgM antibodies or IgM-derived binding molecules. A pentameric IgM-derived binding molecule as provided herein includes five bivalent IgM-derived binding units or variants or multimerizing fragments thereof and a functional variant and/or derivative of a J-chain or functional fragment thereof. By a “functional variant, derivative, or fragment” of a J-chain is meant a J-chain variant, derivative, or fragment that remains capable of associating with five IgM-derived binding units to form a pentamer. Each binding unit of the provided IgM-derived binding molecule includes two IgM heavy chain constant regions or multimerizing fragments or variants thereof, where the constant regions are fused to binding polypeptides as described elsewhere herein. As provided herein, a variant and/or derivative J-chain or functional fragment thereof can include one or more single amino acid substitutions, deletions, or insertions that can affect serum half-life of an IgM-derived binding molecule including the J-chain or functional fragment, variant, and/or derivative thereof. The term “one or more single amino acid substitutions, insertions, and deletions” means that each amino acid of the J-chain or functional fragment, variant, and/or derivative thereof amino acid sequence can individually be substituted, deleted, or can have a single amino acid inserted adjacent thereto, but the J-chain or functional fragment, variant, and/or derivative thereof must still be able to serve the function of assembling with IgM heavy chains or IgM-derived heavy chains to form an IgM-derived pentameric binding molecule. In certain embodiments the J-chain or functional fragment, variant, and/or derivative thereof as provided herein can have a single amino acid substitution, insertion or deletion, a combination of two single amino acid substitutions, insertions, or deletions (e.g., two single amino acid substitutions or one single amino acid substitution and one single amino acid insertion or deletion), a combination of three single amino acid substitutions, insertions, or deletions, a combination of four single amino acid substitutions, insertions, or deletions or more, where the one, two, three, four, or more single amino acid substitutions, insertions or deletions can affect the serum half-life of an IgM-derived binding molecule that includes the J-chain or functional fragment, variant, and/or derivative thereof. Accordingly, the provided IgM-derived binding molecule exhibits an increased serum half-life upon administration to an animal relative to a reference IgM-derived binding molecule that is identical, except for the one or more single amino acid substitutions, deletions, or insertions in the J-chain or functional fragment, variant, and/or derivative thereof, where both the provided binding molecule and the reference binding molecule are administered in the same way to the same animal species. Modified or variant J-chains that can improve the serum half-life of an IgM-derived binding molecule as provided herein are disclosed herein, as well as methods of making and using such J-chains, are disclosed in PCT Publication No. WO 2019/169314, the contents of which is incorporated herein by reference in its entirety.

In certain embodiments, the serum half-life of the IgM-derived binding molecule, e.g., the α half-life, the β half-life, or the overall half-life, can be increased by at least 0.1-fold, at least 0.5-fold, at least 1-fold, at least 5-fold, at least 10-fold, at least 20-fold, at least 30-fold, at least 40-fold, at least 50-fold, at least 60-fold, at least 70-fold, at least 80-fold, at least 90-fold, at least 100-fold, at least 500-fold, at least 1000-fold or more over the reference binding molecule.

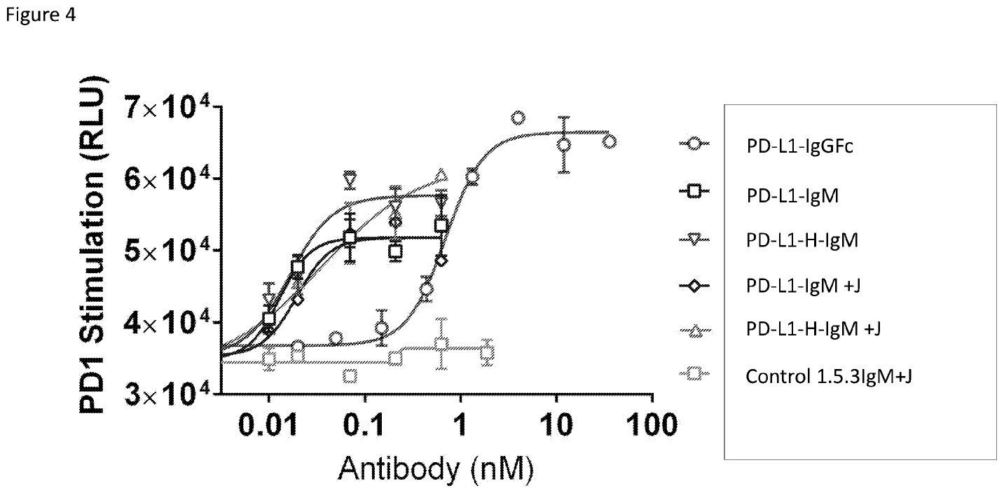

In certain embodiments, the J-chain of the IgM-derived binding molecule as provided herein includes an amino acid substitution at the amino acid position corresponding to amino acid Y102 of the mature human J-chain (SEQ ID NO: 15). By “an amino acid corresponding to amino acid Y102 of the wild-type human J-chain” is meant the amino acid in the sequence of the J-chain of any species which is homologous to Y102 in the human J-chain. The position corresponding to Y102 in SEQ ID NO: 15 is conserved in the J-chain amino acid sequences of at least 43 other species. See FIG. 4 of U.S. Pat. No. 9,951,134, which is incorporated by reference herein. While not wishing to be bound by theory, this mutation is believed to affect the binding of certain immunoglobulin receptors, e.g., the Fcαμ receptor and/or the polymeric Ig receptor (pIg receptor). In certain embodiments, Y102 of SEQ ID NO: 15 can be substituted with any amino acid. In certain embodiments, Y102 of SEQ ID NO: 15 can be substituted with alanine (A), serine (S) or arginine (R). In a particular embodiment, Y102 of SEQ ID NO: 15 can be substituted with alanine. In one embodiment the J-chain or functional fragment, variant, and/or derivative thereof is a variant human J-chain and includes the amino acid sequence SEQ ID NO: 16.

In certain embodiments, the J-chain or functional fragment, variant, and/or derivative thereof of the IgM-derived binding molecule as provided herein includes an amino acid substitution at the amino acid position corresponding to amino acid N49 or amino acid S51 of the mature human J-chain (SEQ ID NO: 15), where S51 is not substituted with threonine (T) or where the J-chain includes amino acid substitutions at the amino acid positions corresponding to both amino acids N49 and S51 of the human J-chain (SEQ ID NO: 15). Again, by “an amino acid corresponding to amino acid N49 of SEQ ID NO: 15 of an amino acid corresponding to S51 of SEQ ID NO: 15 of the wild-type human J-chain” is meant the amino acid in the sequence of the J-chain of any species which is homologous to N49 and/or S51 in the human J-chain. The positions corresponding to N49 and S51 in SEQ ID NO: 15 are conserved in the J-chain amino acid sequences of at least 43 other species. See FIG. 4 of PCT Publication No. WO 2015/153912 which is incorporated by reference herein. While not wishing to be bound by theory, it is believed that the amino acids corresponding to N49 and S51 of SEQ ID NO: 15 along with the amino acid corresponding to ISO of SEQ ID NO: 15 include an N-linked glycosylation motif in the J-chain, and that the mutations at N49 and/or S51 (with the exception of a threonine substitution at S51) can prevent glycosylation at this motif. In certain embodiments, the asparagine at the position corresponding to N49 of SEQ ID NO: 15 can be substituted with any amino acid. In certain embodiments, the asparagine at the position corresponding to N49 of SEQ ID NO: 15 can be substituted with alanine (A), glycine (G), threonine (T), serine (S) or aspartic acid (D). In a particular embodiment the position corresponding to N49 of SEQ ID NO: 15 can be substituted with alanine (A). In a particular embodiment the J-chain is a variant human J-chain and includes the amino acid sequence SEQ ID NO: 17.

In certain embodiments, the serine at the position corresponding to S51 of SEQ ID NO: 15 can be substituted with any amino acid. In certain embodiments, the serine at the position corresponding to S51 of SEQ ID NO: 15 can be substituted with alanine (A) or glycine (G). In a particular embodiment the position corresponding to S51 of SEQ ID NO: 15 can be substituted with alanine (A). In a particular embodiment the J-chain or functional fragment, variant, and/or derivative thereof is a variant human J-chain and includes the amino acid sequence SEQ ID NO: 18.

In certain embodiments, an IgM-derived binding molecule with improved serum half-life as provided herein further exhibits other modified pharmacokinetic parameters, e.g., an increased peak plasma concentration (Cₘₐₓ), an increased area under the curve (AUC), a reduced clearance time, or any combination thereof relative to the reference binding molecule.

In certain embodiments, a J-chain or functional fragment, variant, and/or derivative thereof of an IgM-derived binding molecule as provided herein can be a modified J-chain, e.g., as provided, e.g., in U.S. Pat. No. 9,951,134. In certain embodiments the modified J-chain further includes a heterologous polypeptide, where the heterologous polypeptide is directly or indirectly fused to the J-chain or functional fragment, variant, and/or derivative thereof. In certain embodiment, the heterologous polypeptide is fused to the J-chain or functional fragment, variant, and/or derivative thereof via a peptide linker, e.g., a peptide linker consisting of least 5 amino acids, but no more than 25 amino acids. In certain embodiments, the peptide linker consists of GGGGS (SEQ ID NO: 19), GGGGSGGGGS (SEQ ID NO: 20), GGGGSGGGGSGGGGS (SEQ ID NO: 21), GGGGSGGGGSGGGGSGGGGS (SEQ ID NO: 22), or GGGGSGGGGSGGGGSGGGGSGGGGS (SEQ ID NO: 23). The heterologous polypeptide can be fused to the N-terminus of the J-chain or functional fragment, variant, and/or derivative thereof, the C-terminus of the J-chain or functional fragment, variant, and/or derivative thereof, or heterologous polypeptides can be fused to both the N-terminus and C-terminus of the J-chain or functional fragment, variant, and/or derivative thereof. In certain embodiments, the heterologous polypeptide includes a binding domain. In certain embodiments, the binding domain of the heterologous polypeptide is an antibody or antigen-binding fragment thereof, e.g., a Fab fragment, a Fab′ fragment, an F(ab′)2 fragment, an Fd fragment, an Fv fragment, a single-chain Fv (scFv) fragment, a disulfide-linked Fv (sdFv) fragment, or any combination thereof.

In certain embodiments, a heterologous polypeptide fused to a modified J-chain can include one or more binding polypeptides as provided herein. In certain embodiments, a heterologous polypeptide fused to a modified J-chain can include a polypeptide that influences the absorption, distribution, metabolism and/or excretion (ADME) of the multimeric binding molecule. Exemplary heterologous polypeptides for fusion to a modified J-chain as provided herein are disclosed, e.g., in U.S. Pat. Nos. 9,951,134 and 10,400,038, in U.S. Patent Application Publication Nos. US-2019-0185570 and US-2018-0265596, each of which is incorporated herein by reference in its entirety.

In one embodiment, a heterologous polypeptide fused to a modified J-chain can target a binding molecule as provided herein to a specific cell, tissue, or organ. For example, a pentameric binding molecule that includes a modified J-chain as provided herein can include at least three, at least four, at least five, at least six, at least seven, at least eight, at least nine, or ten VEGF-R2 receptor ectodomain binding polypeptides as described above for binding to and inhibiting VEGF, and a modified J-chain that includes a polypeptide that targets the eye, e.g., a hyaluronic acid binding peptide (HABP), e.g., the LINK domain of Tumor necrosis factor-inducible gene 6 protein, e.g., amino acids 36-129 of SEQ ID NO: 58. See, e.g., Ghosh, J G et al., Nature Comm. 8:14837 (2017). Such a multimeric binding molecule can be used to treat degenerative eye diseases, e.g., age-related macular degeneration.

Another example of tissue targeting is the synovial endothelium targeting peptide (SvETP, CKSTHDRLC, SEQ ID NO: 59) to target synovium. See, e.g., Wythe, S E et al., Ann. Rheum Dis. 72:129-135 (2013).

In another embodiment, a heterologous polypeptide fused to a modified J-chain of a multimeric binding molecule as provided herein can be an scFv antibody fragment that targets an immune checkpoint blockade, e.g., PD-L1, in a multimeric binding molecule including at least three, at least four, at least five, at least six, at least seven, at least eight, at least nine, or ten TGF-β-receptor ectodomain binding polypeptides as described above for binding to and inhibiting TGF-β, to block tumor-induced immunosuppression in the tumor microenvironment. See, e.g., Knudson, K M, et al, Oncoimmunology 7:1426519; DOI:10.1080/2162402X.2018.1426519 (2018). A variety of anti-PD-L1 antibodies are known in the art. Exemplary anti-PD-L1 antibodies are described, e.g., in PCT Publication No. WO/2017/196867, which is incorporated herein by reference in its entirety.

Hexameric and Pentameric IgM-Derived Binding Molecules that Include PD-L1-Binding Polypeptides

In certain embodiments, a binding molecule as provided herein includes ten or twelve IgM-derived heavy chain constant regions or multimerizing fragments thereof, where at least three, at least four, at least five, at least six, at least seven, at least eight, at least nine, at least ten, at least eleven, or twelve of the IgM-derived heavy chain constant regions are fused to a binding polypeptide that includes the V-type and C2-type ectodomains of a PD-L1 protein, e.g., a human PD-L1 protein, e.g., a binding polypeptide that includes the amino acid sequence SEQ ID NO: 9.

In certain embodiments, the IgM-derived heavy chain constant regions include the wild-type Cμ2, Cμ3, Cμ4, and tp domains of the human IgM constant region. For example, in certain embodiments a binding molecule as provided herein includes at least three, at least four, at least five, at least six, at least seven, at least eight, at least nine, at least ten, at least eleven, or twelve copies of a polypeptide that includes amino acids 19 to 587 of SEQ ID NO: 10. Precursors of the component polypeptides of the binding molecule can further include a signal peptide to facilitate secretion of the proteins, e.g., the polypeptides can include the amino acid sequence SEQ ID NO: 10. In certain embodiments, the IgM-derived heavy chain constant regions include a modified human IgM-derived constant region, where the modifications reduce or abrogate CDC activity of the binding molecule, where the IgM-derived heavy chain constant regions include the Cμ2, Cμ3, Cμ4 and tp domains of the human IgM constant region with P311A and P313S amino acid substitutions in the Cμ3 domain. For example, in certain embodiments a binding molecule as provided herein includes at least three, at least four, at least five, at least six, at least seven, at least eight, at least nine, at least ten, at least eleven, or twelve copies of a polypeptide that includes amino acids 19 to 587 of SEQ ID NO: 11. Precursors of the component polypeptides of the binding molecule can further include a signal peptide to facilitate secretion of the proteins, e.g., the polypeptides can include the amino acid sequence SEQ ID NO: 11.

In certain embodiments, the IgM-derived heavy chain constant regions include a modified IgG hinge region, and the wild-type Cμ3, Cμ4, and tp domains of the human IgM constant region. For example, in certain embodiments a binding molecule as provided herein includes at least three, at least four, at least five, at least six, at least seven, at least eight, at least nine, at least ten, at least eleven, or twelve copies of a polypeptide that includes amino acids 19 to 493 of SEQ ID NO: 12. Precursors of the component polypeptides of the binding molecule can further include a signal peptide to facilitate secretion of the proteins, e.g., the polypeptides can include the amino acid sequence SEQ ID NO: 12. In certain embodiments, the IgM-derived heavy chain constant regions include a modified IgG hinge region and a modified human IgM-derived constant region, where the modifications reduce or abrogate CDC activity of the binding molecule, including the modified IgG hinge region, and Cμ3, Cμ4 and tp domains of the human IgM constant region with P311A and P313S amino acid substitutions in the Cμ3 domain. For example, in certain embodiments a binding molecule as provided herein includes at least three, at least four, at least five, at least six, at least seven, at least eight, at least nine, at least ten, at least eleven, or twelve copies of a polypeptide that includes amino acids 19 to 493 of SEQ ID NO: 13. Precursors of the component polypeptides of the binding molecule can further comprise a signal peptide to facilitate secretion of the proteins, e.g., the polypeptides can comprise the amino acid sequence SEQ ID NO: 13.

Where the binding molecule that includes PD-L1 binding polypeptides is pentameric, the binding molecule can further include a J-chain, or functional fragment, variant, or derivative thereof. For example, the binding molecule can include a wild-type human J-chain that includes the amino acid sequence SEQ ID NO: 15, or a variant J-chain that includes one or more amino acid substitutions that affect, e.g., increase or prolong serum half-life of the binding molecule, e.g., a variant human J-chain that includes the amino acid sequence SEQ ID NO: 16, SEQ ID NO: 17, or SEQ ID NO: 18. In a particular embodiment the binding molecule includes ten copies of an amino acid sequence that includes amino acids 19 to 587 of SEQ ID NO: 11 or ten copies of an amino acid sequence that includes amino acids 19 to 493 of SEQ ID NO: 13, and a variant J-chain that includes the amino acid sequence SEQ ID NO: 16.

Hexameric and pentameric IgM-derived binding molecules that include PD-L1-binding polypeptides as provided herein can bind to multiple copies of the binding partner, PD-1 expressed, e.g., on T-cells, thereby effecting signal transduction through PD-1. Accordingly, these binding molecules can function as agonists of PD-1 signal transduction. Such binding molecules can be useful, for example, in treating autoimmune disorders and/or inflammatory disorders, or for the prevention of transplant rejection. Engagement of PD-1 by monomeric or dimeric PD-L1 ectodomain-IgG fc fusion proteins has been demonstrated to inhibit T-cell receptor-mediated lymphocyte proliferation and cytokine secretion. See, e.g., Freeman, G. J. et al., J. Exp. Med. 192:1027-1034 (2000). A multimeric binding molecule as provided herein can function as a PD-1 agonist at higher potency, e.g., 5-fold, 10-fold, 20-fold, 30-fold, 40-fold, 50-fold, 60-fold, 70-fold, 80-fold, 90-fold, 100-fold, 500-fold or 1000-fold higher potency than an equivalent (e.g., molar equivalent or weight equivalent) amount of a monomeric or dimeric PD-L1 ectodomain-based binding molecule, e.g., a binding molecule that includes a PD-L1 ectodomain fused to an IgG Fc region.

Polynucleotides, Vectors, and Host Cells

The disclosure further provides a polynucleotide, e.g., an isolated, recombinant, and/or non-naturally-occurring polynucleotide that includes a nucleic acid sequence that encodes a polypeptide subunit of multimeric binding molecule as described herein. By “polypeptide subunit” is meant a portion of a binding molecule, binding unit, or IgM- or IgA-derived heavy chain fusion protein that can be independently translated. Examples include, without limitation, a fusion protein that includes an IgA or IgM heavy chain constant region or multimerizing fragment or variant thereof fused to a binding polypeptide or fragment thereof, a J chain, a secretory component, or any variant and/or derivative thereof as described herein.

In certain embodiments, the polypeptide subunit can include an IgM or IgM-like heavy chain constant region or multimerizing fragment and/or fragment thereof fused to a binding polypeptide as described herein. In certain embodiments the polynucleotide can encode a polypeptide subunit that includes a human IgM or IgM-like constant region or multimerizing fragment and/or variant thereof fused to the C-terminal end of a binding polypeptide.

In brief, nucleic acid sequences encoding polypeptide subunits of a multimeric binding molecule as provided herein can be synthesized or amplified from existing molecules and inserted into one or more vectors in the proper orientation and in frame such that upon expression, the vector will yield a full-length polypeptide subunit. Vectors useful for these purposes are known in the art. Such vectors can also include enhancer and other sequences needed to achieve expression of the desired chains. Multiple vectors or single vectors can be used and can further encode the J-chain or functional fragment, variant, and/or derivative thereof. This vector or these vectors can be transfected into host cells and then the IgM- or IgA-derived fusion proteins as provided herein and the J-chain or functional fragment, variant, and/or derivative thereof are expressed, the multimeric binding molecule is assembled, and purified. Upon expression the chains form fully functional multimeric binding molecules as provided herein. The fully assembled multimeric binding molecules can then be purified by standard methods. The expression and purification processes can be performed at commercial scale, if needed.

The disclosure further provides a composition that includes two or more polynucleotides, where the two or more polynucleotides collectively can encode a multimeric binding molecule as provided herein. In certain embodiments the composition can include a polynucleotide encoding an IgA or IgM heavy chain constant region or multimerizing fragment or variant thereof fused to a binding polypeptide or fragment thereof, and a polynucleotide encoding a J-chain or functional fragment, variant, and/or derivative thereof. In certain embodiments the polynucleotides making up a composition as provided herein can be situated on two separate vectors, e.g., expression vectors. Such vectors are provided by the disclosure. In certain embodiments two or more of the polynucleotides making up a composition as provided herein can be situated on a single vector, e.g., an expression vector. Such a vector is provided by the disclosure.

The disclosure further provides a host cell, e.g., a prokaryotic or eukaryotic host cell, that includes a polynucleotide or two or more polynucleotides encoding a multimeric binding molecule as provided herein, or any subunit thereof, a polynucleotide composition as provided herein, or a vector or two, three, or more vectors that collectively encode a multimeric binding molecule as provided herein, or any subunit thereof.

In a related embodiment, the disclosure provides a method of producing a multimeric binding molecule as provided by this disclosure, where the method includes culturing a host cell as provided herein and recovering the multimeric binding molecule.

Methods of Use

The disclosure further provides a method of treating a disease or disorder in a subject in need of treatment, where the method includes administering to the subject a therapeutically effective amount of a multimeric binding molecule as provided herein. By “therapeutically effective dose or amount” or “effective amount” is intended an amount of a multimeric binding molecule, that when administered brings about a positive immunotherapeutic response with respect to treatment of subject.

Effective doses of compositions for treatment of a disease or disorder vary depending upon many different factors, including means of administration, target site, physiological state of the subject, whether the subject is human or an animal, other medications administered, and whether treatment is prophylactic or therapeutic. Usually, the subject is a human, but non-human mammals including transgenic mammals can also be treated. Treatment dosages can be titrated using routine methods known to those of skill in the art to optimize safety and efficacy.

In certain embodiments, the disclosure provides a method for treating an autoimmune disorder, an inflammatory disorder, or a combination thereof in a subject in need of treatment, where the method includes administering to the subject an effective amount of a multimeric binding molecule as provided herein. In certain embodiments, administration of a multimeric binding molecule as provided herein to a subject results in greater potency than administration of an equivalent amount of a monomeric or dimeric binding molecule binding to the same binding partner. In certain embodiments the monomeric or dimeric binding molecule includes identical binding polypeptides to the multimeric binding molecule as provided herein. By “an equivalent amount” is meant, e.g., an amount measured by molecular weight, e.g., in total milligrams, or alternative, a molar equivalent, e.g., where equivalent numbers of molecules are administered.

In certain embodiments, the autoimmune disease can be, e.g., arthritis, e.g., rheumatoid arthritis, osteoarthritis, or ankylosing spondylitis, multiple sclerosis (MS), inflammatory bowel disease (IBD) e.g., Crohn's disease or ulcerative colitis, or systemic lupus erythematosus (SLE). In certain embodiments the inflammatory disease or disorder can be, e.g., arthritis, e.g., rheumatoid arthritis, or osteoarthritis, or psoriatic arthritis, Lyme disease, SLE, MS, Sjogren's syndrome, asthma, inflammatory bowel disease, ischemia, atherosclerosis, or stroke.

In other embodiments, the disclosure provides a method for preventing transplantation rejection in a transplantation recipient, where the method includes administering to the subject an effective amount of a multimeric binding molecule as provided herein. In certain embodiments, administration of a multimeric binding molecule as provided herein to a subject result in greater potency than administration of an equivalent amount of a monomeric or dimeric binding polypeptide binding to the same binding partner. In certain embodiments the monomeric or dimeric binding molecule includes identical binding polypeptides to the multimeric binding molecule as provided herein. By “an equivalent amount” is meant, e.g., an amount measured by molecular weight, e.g., in total milligrams, or alternative, a molar equivalent, e.g., where equivalent numbers of molecules are administered.

The subject to be treated can be any animal, e.g., mammal, in need of treatment, in certain embodiments, the subject is a human subject.

In its simplest form, a preparation to be administered to a subject is multimeric binding molecule as provided herein administered in a conventional dosage form, which can be combined with a pharmaceutical excipient, carrier or diluent as described elsewhere herein.

A multimeric binding molecule of the disclosure can be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutical Compositions and Administration Methods

Methods of preparing and administering a multimeric binding molecule as provided herein to a subject in need thereof are well known to or are readily determined by those skilled in the art in view of this disclosure. The route of administration of can be, for example, oral, parenteral, by inhalation or topical. The term parenteral as used herein includes, e.g., intravenous, intraarterial, intraperitoneal, intramuscular, subcutaneous, rectal, or vaginal administration. While these forms of administration are contemplated as suitable forms, another example of a form for administration would be a solution for injection, in particular for intravenous, or intraarterial injection or drip. A suitable pharmaceutical composition can include a buffer (e.g. acetate, phosphate or citrate buffer), a surfactant (e.g. polysorbate), optionally a stabilizer agent (e.g. human albumin), etc.

As discussed herein, a multimeric binding molecule as provided herein can be administered in a pharmaceutically effective amount for the treatment of a subject in need thereof. In this regard, it will be appreciated that the disclosed multimeric binding molecule can be formulated so as to facilitate administration and promote stability of the active agent. Pharmaceutical compositions accordingly can include a pharmaceutically acceptable, non-toxic, sterile carrier such as physiological saline, non-toxic buffers, preservatives and the like. A pharmaceutically effective amount of a multimeric binding molecule as provided herein means an amount sufficient to achieve effective binding to a target and to achieve a therapeutic benefit. Suitable formulations are described in Remington's Pharmaceutical Sciences (Mack Publishing Co.) 16th ed. (1980).

Certain pharmaceutical compositions provided herein can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of a multimeric binding molecule that can be combined with carrier materials to produce a single dosage form will vary depending, e.g., upon the subject treated and the particular mode of administration. The composition can be administered as a single dose, multiple doses or over an established period of time in an infusion. Dosage regimens also can be adjusted to provide the optimum desired response (e.g., a therapeutic or prophylactic response).

In keeping with the scope of the present disclosure, a multimeric binding molecule as provided herein can be administered to a subject in need of therapy in an amount sufficient to produce a therapeutic effect. A multimeric binding molecule as provided herein can be administered to the subject in a conventional dosage form prepared by combining the multimeric binding molecule of the disclosure with a conventional pharmaceutically acceptable carrier or diluent according to known techniques. The form and character of the pharmaceutically acceptable carrier or diluent can be dictated by the amount of active ingredient with which it is to be combined, the route of administration and other well-known variables.

This disclosure also provides for the use of a multimeric binding molecule as provided herein in the manufacture of a medicament for treating, preventing, or managing a disease or disorder, e.g., an autoimmune disease, an inflammatory disease, or for preventing transplantation rejection.

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Green and Sambrook, ed. (2012) Molecular Cloning A Laboratory Manual (4th ed.; Cold Spring Harbor Laboratory Press); Sambrook et al., ed. (1992) Molecular Cloning: A Laboratory Manual, (Cold Springs Harbor Laboratory, NY); D. N. Glover and B. D. Hames, eds., (1995) DNA Cloning 2d Edition (IRL Press), Volumes 1-4; Gait, ed. (1990) Oligonucleotide Synthesis (IRL Press); Mullis et al. U.S. Pat. No. 4,683,195; Hames and Higgins, eds. (1985) Nucleic Acid Hybridization (IRL Press); Hames and Higgins, eds. (1984) Transcription And Translation (IRL Press); Freshney (2016) Culture Of Animal Cells, 7th Edition (Wiley-Blackwell); Woodward, J., Immobilized Cells And Enzymes (IRL Press) (1985); Perbal (1988) A Practical Guide To Molecular Cloning; 2d Edition (Wiley-Interscience); Miller and Calos eds. (1987) Gene Transfer Vectors For Mammalian Cells, (Cold Spring Harbor Laboratory); S. C. Makrides (2003) Gene Transfer and Expression in Mammalian Cells (Elsevier Science); Methods in Enzymology, Vols. 151-155 (Academic Press, Inc., N.Y.); Mayer and Walker, eds. (1987) Immunochemical Methods in Cell and Molecular Biology (Academic Press, London); Weir and Blackwell, eds.; and in Ausubel et al. (1995) Current Protocols in Molecular Biology (John Wiley and Sons).

General principles of antibody engineering are set forth, e.g., in Strohl, W. R., and L. M. Strohl (2012), Therapeutic Antibody Engineering (Woodhead Publishing). General principles of protein engineering are set forth, e.g., in Park and Cochran, eds. (2009), Protein Engineering and Design (CDC Press). General principles of immunology are set forth, e.g., in: Abbas and Lichtman (2017) Cellular and Molecular Immunology 9th Edition (Elsevier). Additionally, standard methods in immunology known in the art can be followed, e.g., in Current Protocols in Immunology (Wiley Online Library); Wild, D. (2013), The Immunoassay Handbook 4th Edition (Elsevier Science); Greenfield, ed. (2013), Antibodies, a Laboratory Manual, 2d Edition (Cold Spring Harbor Press); and Ossipow and Fischer, eds., (2014), Monoclonal Antibodies: Methods and Protocols (Humana Press).

All of the references cited above, as well as all references cited herein, are incorporated herein by reference in their entireties.

The following examples are offered by way of illustration and not by way of limitation.

### EXAMPLES

### Example 1: Construction of Multivalent PD-L1-IgM Fusion Proteins

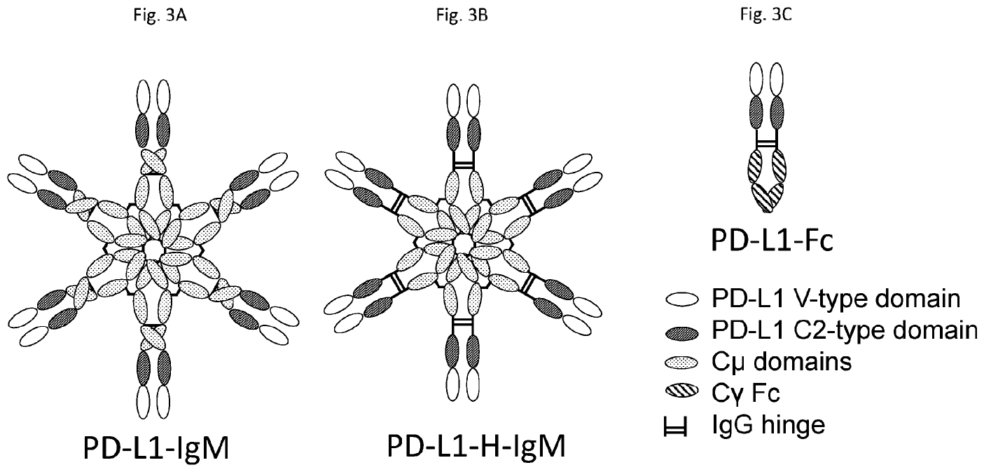

Two DNA constructs encoding PD-L1-IgM fusion protein subunits were constructed by a commercial vendor. Schematics are provided as FIG. 3A and FIG. 3B.

The first construct includes DNA coding for the signal peptide, V1 and C2 domains of human PD-L1 (amino acids 1 to 238 of UniProtKB/Swiss-Prot: Q9NZQ7.1, presented herein as SEQ ID NO: 8), fused to DNA coding for the Cμ2, Cμ3, Cμ4, and tailpiece (tp) domains of a human IgM constant region modified with P311A and P313S amino acid substitutions in the Cμ3 domain in order to reduce or eliminate complement-mediated cytotoxicity (see P PCT Publication No. WO 2018/187702, which is incorporated herein by reference in its entirety). The precursor fusion protein amino acid sequence encoded by the construct is presented herein as SEQ ID NO: 11, and the mature fusion protein amino acid sequence encoded by the construct, following cleavage of the signal peptide (“PD-L1-IgM”), is presented herein as amino acids 19 to 587 of SEQ ID NO: 11. A schematic of a hexameric form of the binding molecule is shown as FIG. 3A.

The second construct includes DNA coding for the signal peptide, V1 and C2 domains of human PD-L1 (amino acids 1 to 238 of UniProtKB/Swiss-Prot: Q9NZQ7.1, presented herein as SEQ ID NO: 8), fused to DNA coding for a variant human IgG2 hinge region (SEQ ID NO: 5) and to DNA coding for the Cμ3, Cμ4, and tailpiece (tp) domains of a human IgM constant region modified with P311A and P313S amino acid substitutions in the Cμ3 domain as above (hinge-modified Cμ3, Cμ4, tp amino acid sequence presented as SEQ ID NO: 6). The precursor fusion protein amino acid sequence encoded by the construct is presented herein as SEQ ID NO: 13, and the mature fusion protein amino acid sequence encoded by the construct, following cleavage of the signal peptide (“PD-L1-H-IgM”), is presented herein as amino acids 19 to 493 of SEQ ID NO: 13. A schematic of a hexameric form of the binding molecule is shown as FIG. 3B.

The resulting DNA constructs were used to transiently transfect Expi293 cells (ThermoFisher) using standard methods. The DNA constructs were either transfected alone to produce hexameric proteins or were cotransfected with a wild-type human J-chain to produce pentameric proteins. The multimeric fusion proteins were purified using the Capture Select IgM affinity matrix (BAC, Thermo Fisher Catalog #2890.05) according to manufacturer's recommendations. The resultant proteins were assessed for proper expression and assembly by non-reducing polyacrylamide gel electrophoresis and western blotting as previously described (see, e.g., PCT Publication No. WO/2018/017888).

A purified fusion protein that includes the ectodomain of human PD-L1 fused to the human IgG1 Fc region (“PD-L1-Fc”) was purchased from R&D Systems (Cat. #156-B7). A schematic of the IgG-Fc construct is shown in FIG. 3C.

### Example 2: Activation of PD-1-Expressing T-Cells by PD-L1-IgM and PD-L1-H-IgM

The ability of hexameric and pentameric versions of the PD-L1-IgM and PD-L1-H-IgM fusion proteins to activate PD-1-expressing T-cells was assessed as follows. Reporter Jurkat T-cells that produce light upon activation through PD-1 were purchased from DiscoverX (PathHunter® PD-1 Assay, Cat. #93-1104C19) and used according to the manufacturer's instructions. In these cells, full-length PD-1 receptor is engineered with a small β-gal fragment fused to its C-terminus, and the SH2-domain of SHP-1 is engineered with the complementing β-gal fragment (EA). These constructs are stably expressed in Jurkat cells. Upon PD-L1 engagement of PD-1 on the surface of these cells, the PD-1 fusion protein is phosphorylated leading to recruitment of the SHP-1 fusion protein which results in an active β-gal enzyme. This active enzyme hydrolyzes substrate to create chemiluminescence as a measure of receptor activity.

These cells were contacted with the monomeric, pentameric, and hexameric constructs purchased or produced as described in Example 1. Jurkat cells with engineered PD-1 and SHP-1 (DiscoverX) were mixed with PD-L1 fusion proteins for 1 h at 37° C. with 5% CO₂. Binding and activation of PD-1 resulted in association of SHP1 to the intracellular domain of PD-L1, which brought together the donor and acceptor components of β-galactosidase. β-galactosidase activity was measured after incubation of chemiluminescence substrate for 3 h at RT in the dark. The results are presented in FIG. 4 and in Table 1.

TABLE 1
EC50s from PD-1 Activation Assay

| Fusion Protein | EC50 in PD-1 Reporter Assay (nM) |
|---|---|
| PD-L1-IgGFc | 0.69 |
| PD-L1-IgM | 0.013 |
| PD-L1-H-IgM | 0.016 |
| PD-L1-IgM + J | 0.019 |
| PD-L1-H-IgM + J | 0.036 |

All of the IgM-based fusion proteins had improved activation EC50s relative to the IgG fusion protein.

TABLE 2
Sequences Presented in the Application

| SEQ ID | | |
|---|---|---|
| NO | Short Name | Sequence |
| 1 | Human IgM | GSASAPTLFPLVSCENSPSDTSSVAVGCLAQDFLPDSITFSW |
| | Constant region | KYKNNSDISSTRGFPSVLRGGKYAATSQVLLPSKDVMQGT |
| | IMGT allele | DEHVVCKVQHPNGNKEKNVPLPVIAELPPKVSVFVPPRDG |
| | IGHM*03 | FFGNPRKSKLICQATGFSPRQIQVSWLREGKQVGSGVTTDQ |
| | | VQAEAKESGPTTYKVTSTLTIKESDWLSQSMFTCRVDHRG |
| | | LTFQQNASSMCVPDQDTAIRVFAIPPSFASIFLTKSTKLTCL |
| | | VTDLTTYDSVTISWTRQNGEAVKTHTNISESHPNATFSAVG |
| | | EASICEDDWNSGERFTCTVTHTDLPSPLKQTISRPKGVALH |
| | | RPDVYLLPPAREQLNLRESATITCLVTGFSPADVFVQWMQ |
| | | RGQPLSPEKYVTSAPMPEPQAPGRYFAHSILTVSEEEWNTG |
| | | ETYTCVVAHEALPNRVTERTVDKSTGKPTLYNVSLVMSDT |
| | | AGTCY |
| 2 | Modified human IgM | GSASAPTLFPLVSCENSPSDTSSVAVGCLAQDFLPDSITFSW |
| | constant region | KYKNNSDISSTRGFPSVLRGGKYAATSQVLLPSKDVMQGT |
| | P311A, P313S | DEHVVCKVQHPNGNKEKNVPLPVIAELPPKVSVFVPPRDG |
| | | FFGNPRKSKLICQATGFSPRQIQVSWLREGKQVGSGVTTDQ |
| | | VQAEAKESGPTTYKVTSTLTIKESDWLSQSMFTCRVDHRG |
| | | LTFQQNASSMCVPDQDTAIRVFAIPPSFASIFLTKSTKLTCL |
| | | VTDLTTYDSVTISWTRQNGEAVKTHTNISESHPNATFSAVG |
| | | EASICEDDWNSGERFTCTVTHTDLASDLKQTISRPKGVALH |
| | | RPDVYLLPPAREQLNLRESATITCLVTGFSPADVFVQWMQ |
| | | RGQPLSPEKYVTSAPMPEPQAPGRYFAHSILTVSEEEWNTG |
| | | ETYTCVVAHEALPNRVTERTVDKSTGKPTLYNVSLVMSDT |
| | | AGTCY |
| 3 | Cmu2,3,4tp human | VIAELPPKVSVFVPPRDGFFGNPRKSKLICQATGFSPRQIQV |
| | WT | SWLREGKQVGSGVTTDQVQAEAKESGPTTYKVTSTLTIKE |
| | | SDWLSQSMFTCRVDHRGLTFQQNASSMCVPDQDTAIRVFA |
| | | IPPSFASIFLTKSTKLTCLVTDLTTYDSVTISWTRQNGEAVK |
| | | THTNISESHPNATFSAVGEASICEDDWNSGERFTCTVTHTD |
| | | LPSPLKQTISRPKGVALHRPDVYLLPPAREQLNLRESATITC |
| | | LVTGFSPADVFVQWMQRGQPLSPEKYVTSAPMPEPQAPGR |
| | | YFAHSILTVSEEEWNTGETYTCVVAHEALPNRVTERTVDK |
| | | STGKPTLYNVSLVMSDTAGTCY* |
| 4 | Cmu2,3,4tp human | VIAELPPKVSVFVPPRDGFFGNPRKSKLICQATGFSPRQIQV |
| | P311A, P313S | SWLREGKQVGSGVTTDQVQAEAKESGPTTYKVTSTLTIKE |
| | | SDWLSQSMFTCRVDHRGLTFQQNASSMCVPDQDTAIRVFA |
| | | IPPSFASIFLTKSTKLTCLVTDLTTYDSVTISWTRQNGEAVK |
| | | THTNISESHPNATFSAVGEASICEDDWNSGERFTCTVTHTD |
| | | LASSLKQTISRPKGVALHRPDVYLLPPAREQLNLRESATITC |
| | | LVTGFSPADVFVQWMQRGQPLSPEKYVTSAPMPEPQAPGR |
| | | YFAHSILTVSEEEWNTGETYTCVVAHEALPNRVTERTVDK |
| | | STGKPTLYNVSLVMSDTAGTCY* |
| 5 | Variant human IgG1 | VEPKSSDKTHTCPPCPAP |
| | hinge region | |
| 6 | H-Cmu3,4tp human | VEPKSSDKTHTCPPCPAPDQDTAIRVFAIPPSFASIFLTKSTK |
| | WT | LTCLVTDLTTYDSVTISWTRQNGEAVKTHTNISESHPNATF |
| | | SAVGEASICEDDWNSGERFTCTVTHTDLPSPLKQTISRPKG |
| | | VALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYNVSL |
| | | VMSDTAGTCY* |
| 7 | H-Cmu3,4tp human | VEPKSSDKTHTCPPCPAPDQDTAIRVFAIPPSFASIFLTKSTK |
| | P311A, P313S | LTCLVTDLTTYDSVTISWTRQNGEAVKTHTNISESHPNATF |
| | | SAVGEASICEDDWNSGERFTCTVTHTDLASSLKQTISRPKG |
| | | VALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYNVSL |
| | | VMSDTAGTCY* |
| 8 | Human PD-L1, | |
| | UniProtKB/Swiss- | MRIFAVFIFMTYWHLLNAFTVTVPKDLYVVEYGSNMTIEC |
| | Prot: Q9NZQ7.1 | KFPVEKQLDLAALIVYWEMEDKNIIQFVHGEEDLKVQHSS |
| | LOCUS | YRQRARLLKDQLSLGNAALQITDVKLQDAGVYRCMISYG |
| | PD1L1_HUMAN 290 | GADYKRITVKVNAPYNKINQRILVVDPVTSEHELTCQAEG |
| | aa | YPKAEVIWTSSDHQVLSGKTTTTNSKREEKLFNVTSTLRIN |
| | | TTTNEIFYCTFRRLDPEENHTAELVIPELPLAHPPNERTHLVI |
| | | LGAILLCLGVALTFIFRLRKGRMMDVKKCGIQDTNSKKQS |
| | | DTHLEET |
| 9 | V-type and C2-type | FTVTVPKDLYVVEYGSNMTIECKFPVEKQLDLAALIVYWE |
| | domains of human | MEDKNIIQFVHGEEDLKVQHSSYRQRARLLKDQLSLGNAA |
| | PD-L1 | LQITDVKLQDAGVYRCMISYGGADYKRITVKVNAPYNKIN |
| | | QRILVVDPVTSEHELTCQAEGYPKAEVIWTSSDHQVLSGKT |
| | | TTTNSKREEKLFNVTSTLRINTTTNEIFYCTFRRLDPEENHT |
| | | AELVIPELPLAHPPNER |
| 10 | PDL1_IgM [V1-C2- | MRIFAVFIFMTYWHLLNAFTVTVPKDLYVVEYGSNMTIEC |
| | Cmu2-Cmu3(WT)- | KFPVEKQLDLAALIVYWEMEDKNIIQFVHGEEDLKVQHSS |
| | Cmu4-TP] w/signal | YRQRARLLKDQLSLGNAALQITDVKLQDAGVYRCMISYG |
| | peptide | GADYKRITVKVNAPYNKINQRILVVDPVTSEHELTCQAEG |
| | | YPKAEVIWTSSDHQVLSGKTTTTNSKREEKLFNVTSTLRIN |
| | | TTTNEIFYCTFRRLDPEENHTAELVIPELPLAHPPNERVIAEL |
| | | PPKVSVFVPPRDGFFGNPRKSKLICQATGFSPRQIQVSWLRE |
| | | GKQVGSGVTTDQVQAEAKESGPTTYKVTSTLTIKESDWLS |
| | | QSMFTCRVDHRGLTFQQNASSMCVPDQDTAIRVFAIPPSFA |
| | | SIFLTKSTKLTCLVTDLTTYDSVTISWTRQNGEAVKTHTNIS |
| | | ESHPNATFSAVGEASICEDDWNSGERFTCTVTHTDLPSPLK |
| | | QTISRPKGVALHRPDVYLLPPAREQLNLRESATITCLVTGFS |
| | | PADVFVQWMQRGQPLSPEKYVTSAPMPEPQAPGRYFAHSI |
| | | LTVSEEEWNTGETYTCVVAHEALPNRVTERTVDKSTGKPT |
| | | LYNVSLVMSDTAGTCY |
| 11 | PDL1_IgM [SP-V1- | MRIFAVFIFMTYWHLLNAFTVTVPKDLYVVEYGSNMTIEC |
| | C2-Cmu2- | KFPVEKQLDLAALIVYWEMEDKNIIQFVHGEEDLKVQHSS |
| | Cmu3(P311A/P313S)- | YRQRARLLKDQLSLGNAALQITDVKLQDAGVYRCMISYG |
| | Cmu4-TP] w/signal | GADYKRITVKVNAPYNKINQRILVVDPVTSEHELTCQAEG |
| | peptide | YPKAEVIWTSSDHQVLSGKTTTTNSKREEKLFNVTSTLRIN |
| | | TTTNEIFYCTFRRLDPEENHTAELVIPELPLAHPPNERVIAEL |
| | | PPKVSVFVPPRDGFFGNPRKSKLICQATGFSPRQIQVSWLRE |
| | | GKQVGSGVTTDQVQAEAKESGPTTYKVTSTLTIKESDWLS |
| | | QSMFTCRVDHRGLTFQQNASSMCVPDQDTAIRVFAIPPSFA |
| | | SIFLTKSTKLTCLVTDLTTYDSVTISWTRQNGEAVKTHTNIS |
| | | ESHPNATFSAVGEASICEDDWNSGERFTCTVTHTDLASSLK |
| | | QTISRPKGVALHRPDVYLLPPAREQLNLRESATITCLVTGFS |
| | | PADVFVQWMQRGQPLSPEKYVTSAPMPEPQAPGRYFAHSI |
| | | LTVSEEEWNTGETYTCVVAHEALPNRVTERTVDKSTGKPT |
| | | LYNVSLVMSDTAGTCY |
| 12 | PDL1_H_IgM [SP- | FTVTVPKDLYVVEYGSNMTIECKFPVEKQLDLAALIVYWE |
| | V1-C2-H(C220S)- | MEDKNIIQFVHGEEDLKVQHSSYRQRARLLKDQLSLGNAA |
| | Cmu3(WT)-Cmu4- | LQITDVKLQDAGVYRCMISYGGADYKRITVKVNAPYNKIN |
| | TP] w/signal peptide | QRILVVDPVTSEHELTCQAEGYPKAEVIWTSSDHQVLSGKT |
| | | TTTNSKREEKLFNVTSTLRINTTTNEIFYCTFRRLDPEENHT |
| | | AELVIPELPLAHPPNERVEPKSSDKTHTCPPCPAPDQDTAIR |
| | | VFAIPPSFASIFLTKSTKLTCLVTDLTTYDSVTISWTRQNGE |
| | | AVKTHTNISESHPNATFSAVGEASICEDDWNSGERFTCTVT |
| | | HTDLASSLKQTISRPKGVALHRPDVYLLPPAREQLNLRESA |
| | | TITCLVTGFSPADVFVQWMQRGQPLSPEKYVTSAPMPEPQ |
| | | APGRYFAHSILTVSEEEWNTGETYTCVVAHEALPNRVTER |
| | | TVDKSTGKPTLYNVSLVMSDTAGTCY |
| 13 | PDL1_H_IgM [SP- | MRIFAVFIFMTYWHLLNAFTVTVPKDLYVVEYGSNMTIEC |
| | V1-C2-H(C220S)- | KFPVEKQLDLAALIVYWEMEDKNIIQFVHGEEDLKVQHSS |
| | Cmu3(P311A/P313S)- | YRQRARLLKDQLSLGNAALQITDVKLQDAGVYRCMISYG |
| | Cmu4-TP] w/signal | GADYKRITVKVNAPYNKINQRILVVDPVTSEHELTCQAEG |
| | peptide | YPKAEVIWTSSDHQVLSGKTTTTNSKREEKLFNVTSTLRIN |
| | | TTTNEIFYCTFRRLDPEENHTAELVIPELPLAHPPNERVEPKS |
| | | SDKTHTCPPCPAPDQDTAIRVFAIPPSFASIFLTKSTKLTCLV |
| | | TDLTTYDSVTISWTRQNGEAVKTHTNISESHPNATFSAVGE |
| | | ASICEDDWNSGERFTCTVTHTDLASSLKQTISRPKGVALHR |
| | | PDVYLLPPAREQLNLRESATITCLVTGFSPADVFVQWMQR |
| | | GQPLSPEKYVTSAPMPEPQAPGRYFAHSILTVSEEEWNTGE |
| | | TYTCVVAHEALPNRVTERTVDKSTGKPTLYNVSLVMSDTA |
| | | GTCY |
| 14 | Precursor Human J | MKNHLLFWGVLAVFIKAVHVKAQEDERIVLVDNKCKCAR |
| | Chain | ITSRIIRSSEDPNEDIVERNIRIIVPLNNRENISDPTSPLRTRFV |
| | | YHLSDLCKKCDPTEVELDNQIVTATQSNICDEDSATETCYT |
| | | YDRNKCYTAVVPLVYGGETKMVETALTPDACYPD |
| 15 | Mature Human J | QEDERIVLVDNKCKCARITSRIIRSSEDPNEDIVERNIRIIVPL |
| | Chain | NNRENISDPTSPLRTRFVYHLSDLCKKCDPTEVELDNQIVT |
| | | ATQSNICDEDSATETCYTYDRNKCYTAVVPLVYGGETKM |
| | | VETALTPDACYPD |
| 16 | Y102A mutation | QEDERIVLVDNKCKCARITSRIIRSSEDPNEDIVERNIRIIVPL |
| | | NNRENISDPTSPLRTRFVYHLSDLCKKCDPTEVELDNQIVT |
| | | ATQSNICDEDSATETCATYDRNKCYTAVVPLVYGGETKM |
| | | VETALTPDACYPD |
| 17 | N49A mutation | QEDERIVLVDNKCKCARITSRIIRSSEDPNEDIVERNIRIIVPL |
| | | NNREAISDPTSPLRTRFVYHLSDLCKKCDPTEVELDNQIVT |
| | | ATQSNICDEDSATETCYTYDRNKCYTAVVPLVYGGETKM |
| | | VETALTPDACYPD |
| 18 | S51A mutation | QEDERIVLVDNKCKCARITSRIIRSSEDPNEDIVERNIRIIVPL |
| | | NNRENIADPTSPLRTRFVYHLSDLCKKCDPTEVELDNQIVT |
| | | ATQSNICDEDSATETCYTYDRNKCYTAVVPLVYGGETKM |
| | | VETALTPDACYPD |
| 19 | 5-linker | GGGGS |
| 20 | 10-linker | GGGGSGGGGS |
| 21 | 15-linker | GGGGSGGGGSGGGGS |
| 22 | 20-linker | GGGGSGGGGSGGGGSGGGGS |
| 23 | 25-linker | GGGGSGGGGSGGGGSGGGGSGGGGS |
| 24 | Human IgA1 Constant | ASPTSPKVFPLSLCSTQPDGNVVIACLVQGFFPQEPLSVTWS |
| | Region | ESGQGVTARNFPPSQDASGDLYTTSSQLTLPATQCLAGKSV |
| | | TCHVKHYTNPSQDVTVPCPVPSTPPTPSPSTPPTPSPSCCHP |
| | | RLSLHRPALEDLLLGSEANLTCTLTGLRDASGVTFTWTPSS |
| | | GKSAVQGPPERDLCGCYSVSSVLPGCAEPWNHGKTFTCTA |
| | | AYPESKTPLTATLSKSGNTFRPEVHLLPPPSEELALNELVTL |
| | | TCLARGFSPKDVLVRWLQGSQELPREKYLTWASRQEPSQG |
| | | TTTFAVTSILRVAAEDWKKGDTFSCMVGHEALPLAFTQKTI |
| | | DRLAGKPTHVNVSVVMAEVDGTCY |
| 25 | Human IgA2 Constant | ASPTSPKVFPLSLDSTPQDGNVVVACLVQGFFPQEPLSVTW |
| | Region | SESGQNVTARNFPPSQDASGDLYTTSSQLTLPATQCPDGKS |
| | | VTCHVKHYTNPSQDVTVPCPVPPPPPCCHPRLSLHRPALED |
| | | LLLGSEANLTCTLTGLRDASGATFTWTPSSGKSAVQGPPER |
| | | DLCGCYSVSSVLPGCAQPWNHGETFTCTAAHPELKTPLTA |
| | | NITKSGNTFRPEVHLLPPPSEELALNELVTLTCLARGFSPKD |
| | | VLVRWLQGSQELPREKYLTWASRQEPSQGTTTFAVTSILR |
| | | VAAEDWKKGDTFSCMVGHEALPLAFTQKTIDRLAGKPTH |
| | | VNVSVVMAEVDGTCY |
| 26 | Human Secretory | MLLFVLTCLLAVFPAISTKSPIFGPEEVNSVEGNSVSITCYYP |
| | Component Precursor | PTSVNRHTRKYWCRQGARGGCITLISSEGYVSSKYAGRAN |
| | | LTNFPENGTFVVNIAQLSQDDSGRYKCGLGINSRGLSFDVS |
| | | LEVSQGPGLLNDTKVYTVDLGRTVTINCPFKTENAQKRKS |
| | | LYKQIGLYPVLVIDSSGYVNPNYTGRIRLDIQGTGQLLFSVV |
| | | INQLRLSDAGQYLCQAGDDSNSNKKNADLQVLKPEPELVY |
| | | EDLRGSVTFHCALGPEVANVAKFLCRQSSGENCDVVVNTL |
| | | GKRAPAFEGRILLNPQDKDGSFSVVITGLRKEDAGRYLCGA |
| | | HSDGQLQEGSPIQAWQLFVNEESTIPRSPTVVKGVAGGSVA |
| | | VLCPYNRKESKSIKYWCLWEGAQNGRCPLLVDSEGWVKA |
| | | QYEGRLSLLEEPGNGTFTVILNQLTSRDAGFYWCLTNGDTL |
| | | WRTTVEIKIIEGEPNLKVPGNVTAVLGETLKVPCHFPCKFSS |
| | | YEKYWCKWNNTGCQALPSQDEGPSKAFVNCDENSRLVSL |
| | | TLNLVTRADEGWYWCGVKQGHFYGETAAVYVAVEERKA |
| | | AGSRDVSLAKADAAPDEKVLDSGFREIENKAIQDPRLFAEE |
| | | KAVADTRDQADGSRASVDSGSSEEQGGSSRALVSTLVPLG |
| | | LVLAVGAVAVGVARARHRKNVDRVSIRSYRTDISMSDFEN |
| | | SREFGANDNMGASSITQETSLGGKEEFVATTESTTETKEPK |
| | | KAKRSSKEEAEMAYKDFLLQSSTVAAEAQDGPQEA |
| 27 | human secretory | KSPIFGPEEVNSVEGNSVSITCYYPPTSVNRHTRKYWCRQG |
| | component mature | ARGGCITLISSEGYVSSKYAGRANLTNFPENGTFVVNIAQLS |
| | | QDDSGRYKCGLGINSRGLSFDVSLEVSQGPGLLNDTKVYT |
| | | VDLGRTVTINCPFKTENAQKRKSLYKQIGLYPVLVIDSSGY |
| | | VNPNYTGRIRLDIQGTGQLLFSVVINQLRLSDAGQYLCQAG |
| | | DDSNSNKKNADLQVLKPEPELVYEDLRGSVTFHCALGPEV |
| | | ANVAKFLCRQSSGENCDVVVNTLGKRAPAFEGRILLNPQD |
| | | KDGSFSVVITGLRKEDAGRYLCGAHSDGQLQEGSPIQAWQ |
| | | LFVNEESTIPRSPTVVKGVAGGSVAVLCPYNRKESKSIKYW |
| | | CLWEGAQNGRCPLLVDSEGWVKAQYEGRLSLLEEPGNGT |
| | | FTVILNQLTSRDAGFYWCLTNGDTLWRTTVEIKIIEGEPNL |
| | | KVPGNVTAVLGETLKVPCHFPCKFSSYEKYWCKWNNTGC |
| | | QALPSQDEGPSKAFVNCDENSRLVSLTLNLVTRADEGWY |
| | | WCGVKQGHFYGETAAVYVAVEERKAAGSRDVSLAKADA |
| | | APDEKVLDSGFREIENKAIQDPR |
| 28 | Human TNF-alpha | >CAA26669.1 TNF-alpha [Homo sapiens] |
| | | MSTESMIRDVELAEEALPKKTGGPQGSRRCLFLSLFSFLIVA |
| | | GATTLFCLLHFGVIGPQREEFPRDLSLISPLAQAVRSSSRTPS |
| | | DKPVAHVVANPQAEGQLQWLNRRANALLANGVELRDNQ |
| | | LVVPSEGLYLIYSQVLFKGQGCPSTHVLLTHTISRIAVSYQT |
| | | KVNLLSAIKSPCQRETPEGAEAKPWYEPIYLGGVFQLEKGD |
| | | RLSAEINRPDYLDFAESGQVYFGIIAL |
| 29 | human TNF-beta | >sp\|P01374.2\|TNFB_HUMAN RecName: Full = Lymphotoxin- |
| | | alpha; Short = LT-alpha; AltName: Full = TNF-beta; AltName: |
| | | Full = Tumor necrosis factor ligand superfamily member 1; Flags: |
| | | Precursor |
| | | MTPPERLFLPRVCGTTLHLLLLGLLLVLLPGAQGLPGVGLT |
| | | PSAAQTARQHPKMHLAHSTLKPAAHLIGDPSKQNSLLWRA |
| | | NTDRAFLQDGFSLSNNSLLVPTSGIYFVYSQVVFSGKAYSP |
| | | KATSSPLYLAHEVQLFSSQYPFHVPLLSSQKMVYPGLQEP |
| | | WLHSMYHGAAFQLTQGDQLSTHTDGIPHLVLSPSTVFFGA |
| | | FAL |
| 30 | human LT-beta | >sp\|Q06643\|TNFC_HUMAN Lymphotoxin-beta OS = Homo |
| | | sapiens OX = 9606 GN = LTB PE = 1 SV = 1 |
| | | MGALGLEGRGGRLQGRGSLLLAVAGATSLVTLLLAVPITV |
| | | LAVLALVPQDQGGLVTETADPGAQAQQGLGFQKLPEEEPE |
| | | TDLSPGLPAAHLIGAPLKGQGLGWETTKEQAFLTSGTQFSD |
| | | AEGLALPQDGLYYLYCLVGYRGRAPPGGGDPQGRSVTLRS |
| | | SLYRAGGAYGPGTPELLLEGAETVTPVLDPARRQGYGPLW |
| | | YTSVGFGGLVQLRRGERVYVNISHPDMVDFARGKTFFGAV |
| | | MVG |
| 31 | Human OX40L | >sp\|P23510.1\|TNFL4_HUMAN RecName: Full = Tumor necrosis |
| | | factor ligand superfamily member 4; AltName: Full = Glycoprotein |
| | | Gp34; AltName: Full = OX40 ligand; Short = OX40L; AltName: |
| | | Full = TAX transcriptionally-activated glycoprotein 1; AltName: |
| | | CD_antigen = CD252 |
| | | MERVQPLEENVGNAARPRFERNKLLLVASVIQGLGLLLCF |
| | | TYICLHFSALQVSHRYPRIQSIKVQFTEYKKEKGFILTSQKE |
| | | DEIMKVQNNSVIINCDGFYLISLKGYFSQEVNISLHYQKDEE |
| | | PLFQLKKVRSVNSLMVASLTYKDKVYLNVTTDNTSLDDFH |
| | | VNGGELILIHQNPGEFCVL |
| 32 | Human CD40L | >sp\|P29965\|CD40L_HUMAN CD40 ligand OS = Homo sapiens |
| | | OX = 9606 GN = CD40LG PE = 1 SV = 1 |
| | | MIETYNQTSPRSAATGLPISMKIFMYLLTVFLITQMIGSALF |
| | | AVYLHRRLDKIEDERNLHEDFVFMKTIQRCNTGERSLSLLN |
| | | CEEIKSQFEGFVKDIMLNKEETKKENSFEMQKGDQNPQIAA |
| | | HVISEASSKTTSVLQWAEKGYYTMSNNLVTLENGKQLTVK |
| | | RQGLYYIYAQVTFCSNREASSQAPFIASLCLKSPGRFERILL |
| | | RAANTHSSAKPCGQQSIHLGGVFELQPGASVFVNVTDPSQ |
| | | VSHGTGFTSFGLLKL |
| 33 | human FasL | >sp\|P48023\|TNFL6_HUMAN Tumor necrosis factor ligand |
| | | superfamily member 6 OS = Homo sapiens OX = 9606 GN = FASLG |
| | | PE = 1 SV = 1 |
| | | MQQPFNYPYPQIYWVDSSASSPWAPPGTVLPCPTSVPRRPG |
| | | QRRPPPPPPPPPLPPPPPPPPLPPLPLPPLKKRGNHSTGLCLLV |
| | | MFFMVLVALVGLGLGMFQLFHLQKELAELRESTSQMHTA |
| | | SSLEKQIGHPSPPPEKKELRKVAHLTGKSNSRSMPLEWEDT |
| | | YGIVLLSGVKYKKGGLVINETGLYFVYSKVYFRGQSCNNL |
| | | PLSHKVYMRNSKYPQDLVMMEGKMMSYCTTGQMWARSS |
| | | YLGAVFNLTSADHLYVNVSELSLVNFEESQTFFGLYKL |
| 34 | human 4-1BB ligand | >sp\|P41273\|TNFL9_HUMAN Tumor necrosis factor ligand |
| | | superfamily member 9 OS = Homo sapiens OX = 9606 |
| | | GN = TNFSF9 PE = 1 SV = 1 |
| | | MEYASDASLDPEAPWPPAPRARACRVLPWALVAGLLLLLL |
| | | LAAACAVFLACPWAVSGARASPGSAASPRLREGPELSPDD |
| | | PAGLLDLRQGMFAQLVAQNVLLIDGPLSWYSDPGLAGVSL |
| | | TGGLSYKEDTKELVVAKAGVYYVFFQLELRRVVAGEGSG |
| | | SVSLALHLQPLRSAAGAAALALTVDLPPASSEARNSAFGFQ |
| | | GRLLHLSAGQRLGVHLHTEARARHAWQLTQGATVLGLFR |
| | | VTPEIPAGLPSPRSE |
| 35 | human TRAIL | >sp\|P50591.1\|TNF10_HUMAN RecName: Full = Tumor necrosis |
| | | factor ligand superfamily member 10; AltName: Full = Apo-2 |
| | | ligand; Short = Apo-2L; AltName: Full = TNF-related apoptosis- |
| | | inducing ligand; Short = Protein TRAIL; AltName: |
| | | CD_antigen = CD253 |
| | | MAMMEVQGGPSLGQTCVLIVIFTVLLQSLCVAVTYVYFTN |
| | | ELKQMQDKYSKSGIACFLKEDDSYWDPNDEESMNSPCWQ |
| | | VKWQLRQLVRKMILRTSEETISTVQEKQQNISPLVRERGPQ |
| | | RVAAHITGTRGRSNTLSSPNSKNEKALGRKINSWESSRSGH |
| | | SFLSNLHLRNGELVIHEKGFYYIYSQTYFRFQEEIKENTKND |
| | | KQMVQYIYKYTSYPDPILLMKSARNSCWSKDAEYGLYSIY |
| | | QGGIFELKENDRIFVSVTNEHLIDMDHEASFFGAFLVG |
| 36 | human GITRL | >sp\|Q9UNG2\|TNF18_HUMAN Tumor necrosis factor ligand |
| | | superfamily member 18 OS = Homo sapiens OX = 9606 |
| | | GN = TNFSF18 PE = 1 SV = 2 |
| | | MTLHPSPITCEFLFSTALISPKMCLSHLENMPLSHSRTQGAQ |
| | | RSSWKLWLFCSIVMLLFLCSFSWLIFIFLQLETAKEPCMAKF |
| | | GPLPSKWQMASSEPPCVNKVSDWKLEILQNGLYLIYGQVA |
| | | PNANYNDVAPFEVRLYKNKDMIQTLTNKSKIQNVGGTYEL |
| | | HVGDTIDLIFNSEHQVLKNNTYWGIILLANPQFIS |
| 37 | Human CD80 | >sp\|P33681\|CD80_HUMAN T-lymphocyte activation antigen |
| | | CD80 OS = Homo sapiens OX = 9606 GN = CD80 PE = 1 SV = 1 |
| | | MGHTRRQGTSPSKCPYLNFFQLLVLAGLSHFCSGVIHVTKE |
| | | VKEVATLSCGHNVSVEELAQTRIYWQKEKKMVLTMMSGD |
| | | MNIWPEYKNRTIFDITNNLSIVILALRPSDEGTYECVVLKYE |
| | | KDAFKREHLAEVTLSVKADFPTPSISDFEIPTSNIRRIICSTSG |
| | | GFPEPHLSWLENGEELNAINTTVSQDPETELYAVSSKLDFN |
| | | MTTNHSFMCLIKYGHLRVNQTFNWNTTKQEHFPDNLLPS |
| | | WAITLISVNGIFVICCLTYCFAPRCRERRRNERLRRESVRPV |
| 38 | Human CD86 | >NP_787058.4 T-lymphocyte activation antigen CD86 isoform 1 |
| | | precursor [Homo sapiens] |
| | | MDPQCTMGLSNILFVMAFLLSGAAPLKIQAYFNETADLPC |
| | | QFANSQNQSLSELVVFWQDQENLVLNEVYLGKEKFDSVHS |
| | | KYMGRTSFDSDSWTLRLHNLQIKDKGLYQCIIHHKKPTGM |
| | | IRIHQMNSELSVLANFSQPEIVPISNITENVYINLTCSSIHGYP |
| | | EPKKMSVLLRTKNSTIEYDGIMQKSQDNVTELYDVSISLSV |
| | | SFPDVTSNMTIFCILETDKTRLLSSPFSIELEDPQPPPDHIPWI |
| | | TAVLPTVIICVMVFCLILWKWKKKKRPRNSYKCGTNTMER |
| | | EESEQTKKREKIHIPERSDEAQRVFKSSKTSSCDKSDTCF |
| 39 | Human DR4 | >sp\|O00220\|TR10A_HUMAN Tumor necrosis factor receptor |
| | | super family member 10A OS = Homo sapiens OX = 9606 |
| | | GN = TNFRSF10A PE = 1 SV = 3 |
| | | MAPPPARVHLGAFLAVTPNPGSAASGTEAAAATPSKVWGS |
| | | SAGRIEPRGGGRGALPTSMGQHGPSARARAGRAPGPRPAR |
| | | EASPRLRVHKTFKFVVVGVLLQVVPSSAATIKLHDQSIGTQ |
| | | QWEHSPLGELCPPGSHRSEHPGACNRCTEGVGYTNASNNL |
| | | FACLPCTACKSDEEERSPCTTTRNTACQCKPGTFRNDNSAE |
| | | MCRKCSRGCPRGMVKVKDCTPWSDIECVHKESGNGHNIW |
| | | VILVVTLVVPLLLVAVLIVCCCIGSGCGGDPKCMDRVCFW |
| | | RLGLLRGPGAEDNAHNEILSNADSLSTFVSEQQMESQEPAD |
| | | LTGVTVQSPGEAQCLLGPAEAEGSQRRRLLVPANGADPTE |
| | | TLMLFFDKFANIVPFDSWDQLMRQLDLTKNEIDVVRAGTA |
| | | GPGDALYAMLMKWVNKTGRNASIHTLLDALERMEERHA |
| | | REKIQDLLVDSGKFIYLEDGTGSAVSLE |
| 40 | Human DR5 | >sp\|O14763\|TR10B_HUMAN Tumor necrosis factor receptor |
| | | superfamily member 10B OS = Homo sapiens OX = 9606 |
| | | GN = TNFRSF10B PE = 1 SV = 2 |
| | | MEQRGQNAPAASGARKRHGPGPREARGARPGPRVPKTLV |
| | | LVVAAVLLLVSAESALITQQDLAPQQRAAPQQKRSSPSEGL |
| | | CPPGHHISEDGRDCISCKYGQDYSTHWNDLLFCLRCTRCDS |
| | | GEVELSPCTTTRNTVCQCEEGTFREEDSPEMCRKCRTGCPR |
| | | GMVKVGDCTPWSDIECVHKESGTKHSGEVPAVEETVTSSP |
| | | GTPASPCSLSGHIGVTVAAVVLIVAVFVCKSLLWKKVLPYL |
| | | KGICSGGGGDPERVDRSSQRPGAEDNVLNEIVSILQPTQVP |
| | | EQEMEVQEPAEPTGVNMLSPGESEHLLEPAEAERSQRRRLL |
| | | VPANEGDPTETLRQCFDDFADLVPFDSWEPLMRKLGLMD |
| | | NEIKVAKAEAAGHRDTLYTMLIKWVNKTGRDASVHTLLD |
| | | ALETLGERLAKQKIEDHLLSSGKFMYLEGNADSAMS |
| 41 | Human OX40 | >sp\|P43489\|TNR4_HUMAN Tumor necrosis factor receptor |
| | | superfamily member 4 OS = Homo sapiens OX = 9606 |
| | | GN = TNFRSF4 PE = 1 SV = 1 |
| | | MCVGARRLGRGPCAALLLLGLGLSTVTGLHCVGDTYPSN |
| | | DRCCHECRPGNGMVSRCSRSQNTVCRPCGPGFYNDVVSSK |
| | | PCKPCTWCNLRSGSERKQLCTATQDTVCRCRAGTQPLDSY |
| | | KPGVDCAPCPPGHFSPGDNQACKPWTNCTLAGKHTLQPAS |
| | | NSSDAICEDRDPPATQPQETQGPPARPITVQPTEAWPRTSQ |
| | | GPSTRPVEVPGGRAVAAILGLGLVLGLLGPLAILLALYLLR |
| | | RDQRLPPDAHKPPGGGSFRTPIQEEQADAHSTLAKI |
| 42 | Human CD40 | >sp\|P25942\|TNR5_HUMAN Tumor necrosis factor receptor |
| | | superfamily member 5 OS = Homo sapiens OX = 9606 GN = CD40 |
| | | PE = 1 SV = 1 |
| | | MVRLPLQCVLWGCLLTAVHPEPPTACREKQYLINSQCCSL |
| | | CQPGQKLVSDCTEFTETECLPCGESEFLDTWNRETHCHQH |
| | | KYCDPNLGLRVQQKGTSETDTICTCEEGWHCTSEACESCV |
| | | LHRSCSPGFGVKQIATGVSDTICEPCPVGFFSNVSSAFEKCH |
| | | PWTSCETKDLVVQQAGTNKTDVVCGPQDRLRALVVIPIIFG |
| | | ILFAILLVLVFIKKVAKKPTNKAPHPKQEPQEINFPDDLPGS |
| | | NTAAPVQETLHGCQPVTQEDGKESRISVQERQ |
| 43 | human 4-1BB | >sp\|Q07011\|TNR9_HUMAN Tumor necrosis factor receptor |
| | | superfamily member 9 OS = Homo sapiens OX = 9606 |
| | | GN = TNFRSF9 PE = 1 SV = 1 |
| | | MGNSCYNIVATLLLVLNFERTRSLQDPCSNCPAGTFCDNN |
| | | RNQICSPCPPNSFSSAGGQRTCDICRQCKGVFRTRKECSSTS |
| | | NAECDCTPGFHCLGAGCSMCEQDCKQGQELTKKGCKDCC |
| | | FGTFNDQKRGICRPWTNCSLDGKSVLVNGTKERDVVCGPS |
| | | PADLSPGASSVTPPAPAREPGHSPQIISFFLALTSTALLFLLFF |
| | | LTLRFSVVKRGRKKLLYIFKQPFMRPVQTTQEEDGCSCRFP |
| | | EEEEGGCEL |
| 44 | human GITR | >sp\|Q9Y5U5\|TNR18_HUMAN Tumor necrosis factor receptor |
| | | superfamily member 18 OS = Homo sapiens OX = 9606 |
| | | GN = TNFRSF18 PE = 1 SV = 1 |
| | | MAQHGAMGAFRALCGLALLCALSLGQRPTGGPGCGPGRL |
| | | LLGTGTDARCCRVHTTRCCRDYPGEECCSEWDCMCVQPE |
| | | FHCGDPCCTTCRHHPCPPGQGVQSQGKFSFGFQCIDCASGT |
| | | FSGGHEGHCKPWTDCTQFGFLTVFPGNKTHNAVCVPGSPP |
| | | AEPLGWLTVVLLAVAACVLLLTSAQLGLHIWQLRSQCMW |
| | | PRETQLLLEVPPSTEDARSCQFPEEERGERSAEEKGRLGDL |
| | | WV |
| 45 | human CTLA4 | >sp\|P16410\|CTLA4_HUMAN Cytotoxic T-lymphocyte protein 4 |
| | | OS = Homo sapiens OX = 9606 GN = CTLA4 PE = 1 SV = 3 |
| | | MACLGFQRHKAQLNLATRTWPCTLLFFLLFIPVFCKAMHV |
| | | AQPAVVLASSRGIASFVCEYASPGKATEVRVTVLRQADSQ |
| | | VTEVCAATYMMGNELTFLDDSICTGTSSGNQVNLTIQGLR |
| | | AMDTGLYICKVELMYPPPYYLGIGNGTQIYVIDPEPCPDSD |
| | | FLLWILAAVSSGLFFYSFLLTAVSLSKMLKKRSPLTTGVYV |
| | | KMPPTEPECEKQFQPYFIPIN |
| 46 | human PD-1 | >sp\|Q15116\|PDCD1_HUMAN Programmed cell death protein 1 |
| | | OS = Homo sapiens OX = 9606 GN = PDCD1 PE = 1 SV = 3 |
| | | MQIPQAPWPVVWAVLQLGWRPGWFLDSPDRPWNPPTFSP |
| | | ALLVVTEGDNATFTCSFSNTSESFVLNWYRMSPSNQTDKL |
| | | AAFPEDRSQPGQDCRFRVTQLPNGRDFHMSVVRARRNDSG |
| | | TYLCGAISLAPKAQIKESLRAELRVTERRAEVPTAHPSPSPR |
| | | PAGQFQTLVVGVVGGLLGSLVLLVWVLAVICSRAARGTIG |
| | | ARRTGQPLKEDPSAVPVFSVDYGELDFQWREKTPEPPVPC |
| | | VPEQTEYATIVFPSGMGTSSPARRGSADGPRSAQPLRPEDG |
| | | HCSWPL |
| 47 | Human LAG3 | >sp\|P18627\|LAG3_HUMAN Lymphocyte activation gene 3 |
| | | protein OS = Homo sapiens OX = 9606 GN = LAG3 PE = 1 SV = 5 |
| | | MWEAQFLGLLFLQPLWVAPVKPLQPGAEVPVVWAQEGAP |
| | | AQLPCSPTIPLQDLSLLRRAGVTWQHQPDSGPPAAAPGHPL |
| | | APGPHPAAPSSWGPRPRRYTVLSVGPGGLRSGRLPLQPRVQ |
| | | LDERGRQRGDFSLWLRPARRADAGEYRAAVHLRDRALSC |
| | | RLRLRLGQASMTASPPGSLRASDWVILNCSFSRPDRPASVH |
| | | WFRNRGQGRVPVRESPHHHLAESFLFLPQVSPMDSGPWGC |
| | | ILTYRDGFNVSIMYNLTVLGLEPPTPLTVYAGAGSRVGLPC |
| | | RLPAGVGTRSFLTAKWTPPGGGPDLLVTGDNGDFTLRLED |
| | | VSQAQAGTYTCHIHLQEQQLNATVTLAIITVTPKSFGSPGSL |
| | | GKLLCEVTPVSGQERFVWSSLDTPSQRSFSGPWLEAQEAQ |
| | | LLSQPWQCQLYQGERLLGAAVYFTELSSPGAQRSGRAPGA |
| | | LPAGHLLLFLILGVLSLLLLVTGAFGFHLWRRQWRPRRFSA |
| | | LEQGIHPPQAQSKIEELEQEPEPEPEPEPEPEPEPEPEQL |
| 48 | human CD28 | >sp\|P10747\|CD28_HUMAN T-cell-specific surface glycoprotein |
| | | CD28 OS = Homo sapiens OX = 9606 GN = CD28 PE = 1 SV = 1 |
| | | MLRLLLALNLFPSIQVTGNKILVKQSPMLVAYDNAVNLSC |
| | | KYSYNLFSREFRASLHKGLDSAVEVCVVYGNYSQQLQVYS |
| | | KTGFNCDGKLGNESVTFYLQNLYVNQTDIYFCKIEVMYPP |
| | | PYLDNEKSNGTIIHVKGKHLCPSPLFPGPSKPFWVLVVVGG |
| | | VLACYSLLVTVAFIIFWVRSKRSRLLHSDYMNMTPRRPGPT |
| | | RKHYQPYAPPRDFAAYRS |
| 49 | Human ILDR2 | >sp\|Q71H61\|ILDR2_HUMAN Immunoglobulin-like domain- |
| | | containing receptor 2 OS = Homo sapiens OX = 9606 GN = ILDR2 |
| | | PE = 2 SV = 1 |
| | | MDRVLLRWISLFWLTAMVEGLQVTVPDKKKVAMLFQPTV |
| | | LRCHFSTSSHQPAVVQWKFKSYCQDRMGESLGMSSTRAQS |
| | | LSKRNLEWDPYLDCLDSRRTVRVVASKQGSTVTLGDFYRG |
| | | REITIVHDADLQIGKLMWGDSGLYYCIITTPDDLEGKNEDS |
| | | VELLVLGRTGLLADLLPSFAVEIMPEWVFVGLVLLGVFLFF |
| | | VLVGICWCQCCPHSCCCYVRCPCCPDSCCCPQALYEAGKA |
| | | AKAGYPPSVSGVPGPYSIPSVPLGGAPSSGMLMDKPHPPPL |
| | | APSDSTGGSHSVRKGYRIQADKERDSMKVLYYVEKELAQF |
| | | DPARRMRGRYNNTISELSSLHEEDSNFRQSFHQMRSKQFPV |
| | | SGDLESNPDYWSGVMGGSSGASRGPSAMEYNKEDRESFR |
| | | HSQPRSKSEMLSRKNFATGVPAVSMDELAAFADSYGQRPR |
| | | RADGNSHEARGGSRFERSESRAHSGFYQDDSLEEYYGQRS |
| | | RSREPLTDADRGWAFSPARRRPAEDAHLPRLVSRTPGTAP |
| | | KYDHSYLGSARERQARPEGASRGGSLETPSKRSAQLGPRS |
| | | ASYYAWSPPGTYKAGSSQDDQEDASDDALPPYSELELTRG |
| | | PSYRGRDLPYHSNSEKKRKKEPAKKTNDFPTRMSLVV |
| 50 | Human TIM-3 | >sp\|Q8TDQ0\|HAVR2_HUMAN Hepatitis A virus cellular |
| | | receptor 2 OS = Homo sapiens OX = 9606 GN = HAVCR2 PE = 1 |
| | | SV = 3 |
| | | MFSHLPFDCVLLLLLLLLTRSSEVEYRAEVGQNAYLPCFYT |
| | | PAAPGNLVPVCWGKGACPVFECGNVVLRTDERDVNYWTS |
| | | RYWLNGDFRKGDVSLTIENVTLADSGIYCCRIQIPGIMNDE |
| | | KFNLKLVIKPAKVTPAPTRQRDFTAAFPRMLTTRGHGPAET |
| | | QTLGSLPDINLTQISTLANELRDSRLANDLRDSGATIRIGIYI |
| | | GAGICAGLALALIFGALIFKWYSHSKEKIQNLSLISLANLPPS |
| | | GLANAVAEGIRSEENIYTIEENVYEVEEPNEYYCYVSSRQQ |
| | | PSQPLGCRFAMP |
| 51 | Human TGFβ | >sp\|P36897\|TGFR1_HUMAN TGF-beta receptor type-1 |
| | receptor-1 (ALK5) | OS = Homo sapiens OX = 9606 GN = TGFBR1 PE = 1 SV = 1 |
| | | MEAAVAAPRPRLLLLVLAAAAAAAAALLPGATALQCFCH |
| | | LCTKDNFTCVTDGLCFVSVTETTDKVIHNSMCIAEIDLIPRD |
| | | RPFVCAPSSKTGSVTTTYCCNQDHCNKIELPTTVKSSPGLG |
| | | PVELAAVIAGPVCFVCISLMLMVYICHNRTVIHHRVPNEED |
| | | PSLDRPFISEGTTLKDLIYDMTTSGSGSGLPLLVQRTIARTIV |
| | | LQESIGKGRFGEVWRGKWRGEEVAVKIFSSREERSWFREA |
| | | EIYQTVMLRHENILGFIAADNKDNGTWTQLWLVSDYHEH |
| | | GSLFDYLNRYTVTVEGMIKLALSTASGLAHLHMEIVGTQG |
| | | KPAIAHRDLKSKNILVKKNGTCCIADLGLAVRHDSATDTID |
| | | IAPNHRVGTKRYMAPEVLDDSINMKHFESFKRADIYAMGL |
| | | VFWEIARRCSIGGIHEDYQLPYYDLVPSDPSVEEMRKVVCE |
| | | QKLRPNIPNRWQSCEALRVMAKIMRECWYANGAARLTAL |
| | | RIKKTLSQLSQQEGIKM |
| 52 | Human TGFβ | >sp\|P37173\|TGFR2_HUMAN TGF-beta receptor type-2 |
| | receptor-2 | OS = Homo sapiens OX = 9606 GN = TGFBR2 PE = 1 SV = 2 |
| | | MGRGLLRGLWPLHIVLWTRIASTIPPHVQKSVNNDMIVTD |
| | | NNGAVKFPQLCKFCDVRFSTCDNQKSCMSNCSITSICEKPQ |
| | | EVCVAVWRKNDENITLETVCHDPKLPYHDFILEDAASPKCI |
| | | MKEKKKPGETFFMCSCSSDECNDNIIFSEEYNTSNPDLLLVI |
| | | FQVTGISLLPPLGVAISVIIIFYCYRVNRQQKLSSTWETGKTR |
| | | KLMEFSEHCAIILEDDRSDISSTCANNINHNTELLPIELDTLV |
| | | GKGRFAEVYKAKLKQNTSEQFETVAVKIFPYEEYASWKTE |
| | | KDIFSDINLKHENILQFLTAEERKTELGKQYWLITAFHAKG |
| | | NLQEYLTRHVISWEDLRKLGSSLARGIAHLHSDHTPCGRPK |
| | | MPIVHRDLKSSNILVKNDLTCCLCDFGLSLRLDPTLSVDDL |
| | | ANSGQVGTARYMAPEVLESRMNLENVESFKQTDVYSMAL |
| | | VLWEMTSRCNAVGEVKDYEPPFGSKVREHPCVESMKDNV |
| | | LRDRGRPEIPSFWLNHQGIQMVCETLTECWDHDPEARLTA |
| | | QCVAERFSELEHLDRLSGRSCSEEKIPEDGSLNTTK |
| 53 | Human TGFβ | >sp\|Q03167\|TGBR3_HUMAN Transforming growth factor beta |
| | receptor-3 | receptor type 3 OS = Homo sapiens OX = 9606 GN = TGFBR3 PE = 1 |
| | | SV = 3 |
| | | MTSHYVIAIFALMSSCLATAGPEPGALCELSPVSASHPVQA |
| | | LMESFTVLSGCASRGTTGLPQEVHVLNLRTAGQGPGQLQR |
| | | EVTLHLNPISSVHIHHKSVVFLLNSPHPLVWHLKTERLATG |
| | | VSRLFLVSEGSVVQFSSANFSLTAETEERNFPHGNEHLLNW |
| | | ARKEYGAVTSFTELKIARNIYIKVGEDQVFPPKCNIGKNFLS |
| | | LNYLAEYLQPKAAEGCVMSSQPQNEEVHIIELITPNSNPYS |
| | | AFQVDITIDIRPSQEDLEVVKNLILILKCKKSVNWVIKSFDV |
| | | KGSLKIIAPNSIGFGKESERSMTMTKSIRDDIPSTQGNLVKW |
| | | ALDNGYSPITSYTMAPVANRFHLRLENNAEEMGDEEVHTI |
| | | PPELRILLDPGALPALQNPPIRGGEGQNGGLPFPFPDISRRV |
| | | WNEEGEDGLPRPKDPVIPSIQLFPGLREPEEVQGSVDIALSV |
| | | KCDNEKMIVAVEKDSFQASGYSGMDVTLLDPTCKAKMNG |
| | | THFVLESPLNGCGTRPRWSALDGVVYYNSIVIQVPALGDSS |
| | | GWPDGYEDLESGDNGFPGDMDEGDASLFTRPEIVVFNCSL |
| | | QQVRNPSSFQEQPHGNITFNMELYNTDLFLVPSQGVFSVPE |
| | | NGHVYVEVSVTKAEQELGFAIQTCFISPYSNPDRMSHYTIIE |
| | | NICPKDESVKFYSPKRVHFPIPQADMDKKRFSFVFKPVFNT |
| | | SLLFLQCELTLCTKMEKHPQKLPKCVPPDEACTSLDASIIW |
| | | AMMQNKKTFTKPLAVIHHEAESKEKGPSMKEPNPISPPIFH |
| | | GLDTLTVMGIAFAAFVIGALLTGALWYIYSHTGETAGRQQ |
| | | VPTSPPASENSSAAHSIGSTQSTPCSSSSTA |
| 54 | Human VEGFR-2 | >sp\|P35968\|VGFR2_HUMAN Vascular endothelial growth factor |
| | | receptor 2 OS = Homo sapiens OX = 9606 GN = KDR PE = 1 SV = 2 |
| | | MQSKVLLAVALWLCVETRAASVGLPSVSLDLPRLSIQKDIL |
| | | TIKANTTLQITCRGQRDLDWLWPNNQSGSEQRVEVTECSD |
| | | GLFCKTLTIPKVIGNDTGAYKCFYRETDLASVIYVYVQDYR |
| | | SPFIASVSDQHGVVYITENKNKTVVIPCLGSISNLNVSLCAR |
| | | YPEKRFVPDGNRISWDSKKGFTIPSYMISYAGMVFCEAKIN |
| | | DESYQSIMYIVVVVGYRIYDVVLSPSHGIELSVGEKLVLNC |
| | | TARTELNVGIDFNWEYPSSKHQHKKLVNRDLKTQSGSEMK |
| | | KFLSTLTIDGVTRSDQGLYTCAASSGLMTKKNSTFVRVHE |
| | | KPFVAFGSGMESLVEATVGERVRIPAKYLGYPPPEIKWYK |
| | | NGIPLESNHTIKAGHVLTIMEVSERDTGNYTVILTNPISKEK |
| | | QSHVVSLVVYVPPQIGEKSLISPVDSYQYGTTQTLTCTVYAI |
| | | PPPHHIHWYWQLEEECANEPSQAVSVTNPYPCEEWRSVED |
| | | FQGGNKIEVNKNQFALIEGKNKTVSTLVIQAANVSALYKC |
| | | EAVNKVGRGERVISFHVTRGPEITLQPDMQPTEQESVSLWC |
| | | TADRSTFENLTWYKLGPQPLPIHVGELPTPVCKNLDTLWK |
| | | LNATMFSNSTNDILIMELKNASLQDQGDYVCLAQDRKTKK |
| | | RHCVVRQLTVLERVAPTITGNLENQTTSIGESIEVSCTASGN |
| | | PPPQIMWFKDNETLVEDSGIVLKDGNRNLTIRRVRKEDEGL |
| | | YTCQACSVLGCAKVEAFFIIEGAQEKTNLEIIILVGTAVIAM |
| | | FFWLLLVIILRTVKRANGGELKTGYLSIVMDPDELPLDEHC |
| | | ERLPYDASKWEFPRDRLKLGKPLGRGAFGQVIEADAFGID |
| | | KTATCRTVAVKMLKEGATHSEHRALMSELKILIHIGHHLN |
| | | VVNLLGACTKPGGPLMVIVEFCKFGNLSTYLRSKRNEFVP |
| | | YKTKGARFRQGKDYVGAIPVDLKRRLDSITSSQSSASSGFV |
| | | EEKSLSDVEEEEAPEDLYKDFLTLEHLICYSFQVAKGMEFL |
| | | ASRKCIHRDLAARNILLSEKNVVKICDFGLARDIYKDPDYV |
| | | RKGDARLPLKWMAPETIFDRVYTIQSDVWSFGVLLWEIFSL |
| | | GASPYPGVKIDEEFCRRLKEGTRMRAPDYTTPEMYQTMLD |
| | | CWHGEPSQRPTFSELVEHLGNLLQANAQQDGKDYIVLPISE |
| | | TLSMEEDSGLSLPTSPVSCMEEEEVCDPKFHYDNTAGISQY |
| | | LQNSKRKSRPVSVKTFEDIPLEEPEVKVIPDDNQTDSGMVL |
| | | ASEELKTLEDRTKLSPSFGGMVPSKSRESVASEGSNQTSGY |
| | | QSGYHSDDTDTTVYSSEEAELLKLIEIGVQTGSTAQILQPDS |
| | | GTTLSSPPV |
| 55 | Human VEGFR-3 | >sp\|P35916\|VGFR3_HUMAN Vascular endothelial growth factor |
| | | receptor 3 OS = Homo sapiens OX = 9606 GN = FLT4 PE = 1 SV = 3 |
| | | MQRGAALCLRLWLCLGLLDGLVSGYSMTPPTLNITEESHVI |
| | | DTGDSLSISCRGQHPLEWAWPGAQEAPATGDKDSEDTGVV |
| | | RDCEGTDARPYCKVLLLHEVHANDTGSYVCYYKYIKARIE |
| | | GTTAASSYVFVRDFEQPFINKPDTLLVNRKDAMWVPCLVSI |
| | | PGLNVTLRSQSSVLWPDGQEVVWDDRRGMLVSTPLLHDA |
| | | LYLQCETTWGDQDFLSNPFLVHITGNELYDIQLLPRKSLEL |
| | | LVGEKLVLNCTVWAEFNSGVTFDWDYPGKQAERGKWVP |
| | | ERRSQQTHTELSSILTIHNVSQHDLGSYVCKANNGIQRFRES |
| | | TEVIVHENPFISVEWLKGPILEATAGDELVKLPVKLAAYPPP |
| | | EFQWYKDGKALSGRHSPHALVLKEVTEASTGTYTLALWN |
| | | SAAGLRRNISLELVVNVPPQIHEKEASSPSIYSRHSRQALTC |
| | | TAYGVPLPLSIQWHWRPWTPCKMFAQRSLRRRQQQDLMP |
| | | QCRDWRAVTTQDAVNPIESLDTWTEFVEGKNKTVSKLVIQ |
| | | NANVSAMYKCVVSNKVGQDERLIYFYVTTIPDGFTIESKPS |
| | | EELLEGQPVLLSCQADSYKYEHLRWYRLNLSTLHDAHGNP |
| | | LLLDCKNVHLFATPLAASLEEVAPGARHATLSLSIPRVAPE |
| | | HEGHYVCEVQDRRSHDKHCHKKYLSVQALEAPRLTQNLT |
| | | DLLVNVSDSLEMQCLVAGAHAPSIVWYKDERLLEEKSGV |
| | | DLADSNQKLSIQRVREEDAGRYLCSVCNAKGCVNSSASVA |
| | | VEGSEDKGSMEIVILVGTGVIAVFFWVLLLLIFCNMRRPAH |
| | | ADIKTGYLSIIMDPGEVPLEEQCEYLSYDASQWEFPRERLH |
| | | LGRVLGYGAFGKVVEASAFGIHKGSSCDTVAVKMLKEGA |
| | | TASEHRALMSELKILIHIGNHLNVVNLLGACTKPQGPLMVI |
| | | VEFCKYGNLSNFLRAKRDAFSPCAEKSPEQRGRFRAMVEL |
| | | ARLDRRRPGSSDRVLFARFSKTEGGARRASPDQEAEDLWL |
| | | SPLTMEDLVCYSFQVARGMEFLASRKCIHRDLAARNILLSE |
| | | SDVVKICDFGLARDIYKDPDYVRKGSARLPLKWMAPESIF |
| | | DKVYTTQSDVWSFGVLLWEIFSLGASPYPGVQINEEFCQRL |
| | | RDGTRMRAPELATPAIRRIMLNCWSGDPKARPAFSELVEIL |
| | | GDLLQGRGLQEEEEVCMAPRSSQSSEEGSFSQVSTMALHIA |
| | | QADAEDSPPSLQRHSLAARYYNWVSFPGCLARGAETRGSS |
| | | RMKTFEEFPMTPTTYKGSVDNQTSGMVLASEEFEQIESRH |
| | | RQESGFSCKGPGQNVAVTRAHPDSQGRRRRPERGARGGQ |
| | | VFYNSEYGELSEPSEEDHCSPSARVTFFTDNSY |
| 56 | Human VEGFR-1 | >sp\|P17948\|VGFR1_HUMAN Vascular endothelial growth factor |
| | | receptor 1 OS = Homo sapiens OX = 9606 GN = FLT1 PE = 1 SV = 2 |
| | | MVSYWDTGVLLCALLSCLLLTGSSSGSKLKDPELSLKGTQ |
| | | HIMQAGQTLHLQCRGEAAHKWSLPEMVSKESERLSITKSA |
| | | CGRNGKQFCSTLTLNTAQANHTGFYSCKYLAVPTSKKKET |
| | | ESAIYIFISDTGRPFVEMYSEIPEIIHMTEGRELVIPCRVTSPNI |
| | | TVTLKKFPLDTLIPDGKRIIWDSRKGFIISNATYKEIGLLTCE |
| | | ATVNGHLYKTNYLTHRQTNTIIDVQISTPRPVKLLRGHTLV |
| | | LNCTATTPLNTRVQMTWSYPDEKNKRASVRRRIDQSNSHA |
| | | NIFYSVLTIDKMQNKDKGLYTCRVRSGPSFKSVNTSVHIYD |
| | | KAFITVKHRKQQVLETVAGKRSYRLSMKVKAFPSPEVVWL |
| | | KDGLPATEKSARYLTRGYSLIIKDVTEEDAGNYTILLSIKQS |
| | | NVFKNLTATLIVNVKPQIYEKAVSSFPDPALYPLGSRQILTC |
| | | TAYGIPQPTIKWFWHPCNHNHSEARCDFCSNNEESFILDAD |
| | | SNMGNRIESITQRMAIIEGKNKMASTLVVADSRISGIYICIAS |
| | | NKVGTVGRNISFYITDVPNGFHVNLEKMPTEGEDLKLSCTV |
| | | NKFLYRDVTWILLRTVNNRTMHYSISKQKMAITKEHSITLN |
| | | LTIMNVSLQDSGTYACRARNVYTGEEILQKKEITIRDQEAP |
| | | YLLRNLSDHTVAISSSTTLDCHANGVPEPQITWFKNNHKIQ |
| | | QEPGHLGPGSSTLFIERVTEEDEGVYHCKATNQKGSVESSA |
| | | YLTVQGTSDKSNLELITLTCTCVAATLFWLLLTLFIRKMKR |
| | | SSSEIKTDYLSIIMDPDEVPLDEQCERLPYDASKWEFARERL |
| | | KLGKSLGRGAFGKVVQASAFGIKKSPTCRTVAVKMLKEG |
| | | ATASEYKALMTELKILTHIGHHLNVVNLLGACTKQGGPLM |
| | | VIVEYCKYGNLSNYLKSKRDLFFLNKDAALHMEPKKEKM |
| | | EPGLEQGKKPRLDSVTSSESFASSGFQEDKSLSDVEEEEDSD |
| | | GFYKEPITMEDLISYSFQVARGMEFLSSRKCIHRDLAARNIL |
| | | LSENNVVKICDFGLARDIYKNPDYVRKGDTRLPLKWMAPE |
| | | SIFDKIYSTKSDVWSYGVLLWEIFSLGGSPYPGVQMDEDFC |
| | | SRLREGMRMRAPEYSTPEIYQIMLDCWHRDPKERPRFAEL |
| | | VEKLGDLLQANVQQDGKDYIPINAILTGNSGFTYSTPAFSE |
| | | DFFKESISAPKFNSGSSDDVRYVNAFKFMSLERIKTFEELLP |
| | | NATSMFDDYQGDSSTLLASPMLKRFTWTDSKPKASLKIDL |
| | | RVTSKSKESGLSDVSRPSFCHSSCGHVSEGKRRFTYDHAEL |
| | | ERKIACCSPPPDYNSVVLYSTPPI |
| 57 | aflibercept | >Protein sequence for aflibercept |
| | | SDTGRPFVEMYSEIPEIIHMTEGRELVIPCRVTSPNITVTLKK |
| | | FPLDTLIPDGKRIIWDSRKGFIISNATYKEIGLLTCEATVNGH |
| | | LYKTNYLTHRQTNTIIDVVLSPSHGIELSVGEKLVLNCTART |
| | | ELNVGIDFNWEYPSSKHQHKKLVNRDLKTQSGSEMKKFLS |
| | | TLTIDGVTRSDQGLYTCAASSGLMTKKNSTFVRVHEKDKT |
| | | HTCPPCPAPELLGGPSVFLFPPKPKDTLMISRTPEVTCVVVD |
| | | VSHEDPEVKFNWYVDGVEVHNAKTKPREEQYNSTYRVVS |
| | | VLTVLHQDWLNGKEYKCKVSNKALPAPIEKTISKAKGQPR |
| | | EPQVYTLPPSRDELTKNQVSLTCLVKGFYPSDIAVEWESNG |
| | | QPENNYKTTPPVLDSDGSFFLYSKLTVDKSRWQQGNVFSC |
| | | SVMHEALHNHYTQKSLSLSPG |
| 58 | TSG6 Hyaluronic acid | >sp\|P98066\|TSG6_HUMAN Tumor necrosis factor-inducible |
| | binding protein | gene 6 protein OS = Homo sapiens OX = 9606 GN = TNFAIP6 PE = 1 |
| | | SV = 2 |
| | | MIILIYLFLLLWEDTQGWGFKDGIFHNSIWLERAAGVYHRE |
| | | ARSGKYKLTYAEAKAVCEFEGGHLATYKQLEAARKIGFH |
| | | VCAAGWMAKGRVGYPIVKPGPNCGFGKTGIIDYGIRLNRS |
| | | ERWDAYCYNPHAKECGGVFTDPKQIFKSPGFPNEYEDNQI |
| | | CYWHIRLKYGQRIHLSFLDFDLEDDPGCLADYVEIYDSYD |
| | | DVHGFVGRYCGDELPDDIISTGNVMTLKFLSDASVTAGGF |
| | | QIKYVAMDPVSKSSQGKNTSTTSTGNKNFLAGRFSHL |
| 59 | synovial | CKSTHDRLC |
| | endothelium | |
| | targeting peptide | |
| | (SvETP) | |
| 60 | Human IgM | GSASAPTLFPLVSCENSPSDTSSVAVGCLAQDFLPDSIT |
| | Constant region | FSWKYKNNSDISSTRGFPSVLRGGKYAATSQVLLPSK |
| | IMGT allele | DVMQGTDEHVVCKVQHPNGNKEKNVPLPVIAELPPK |
| | IGHM*04 | VSVFVPPRDGFFGNPRKSKLICQATGFSPRQIQVSWLR |
| | | EGKQVGSGVTTDQVQAEAKESGPTTYKVTSTLTIKES |
| | | DWLGQSMFTCRVDHRGLTFQQNASSMCVPDQDTAIR |
| | | VFAIPPSFASIFLTKSTKLTCLVTDLTTYDSVTISWTRQ |
| | | NGEAVKTHTNISESHPNATFSAVGEASICEDDWNSGE |
| | | RFTCTVTHTDLPSPLKQTISRPKGVALHRPDVYLLPPA |
| | | REQLNLRESATITCLVTGFSPADVFVQWMQRGQPLSP |
| | | EKYVTSAPMPEPQAPGRYFAHSILTVSEEEWNTGETY |
| | | TCVVAHEALPNRVTERTVDKSTGKPTLYNVSLVMSD |
| | | TAGTCY |

The breadth and scope of the present disclosure should not be limited by any of the above-described exemplary embodiments but should be defined only in accordance with the following claims and their equivalents.

What is claimed is:

1. A multimeric binding molecule comprising two, five, or six bivalent binding units or variants or fragments thereof,
wherein each binding unit comprises two IgA or IgM heavy chain constant regions or multimerizing fragments or variants thereof, each fused to a binding polypeptide or fragment thereof that specifically binds to a binding partner expressed on the surface of a cell, wherein the binding polypeptide is not an antibody or antigen-binding fragment of an antibody, and wherein binding of the binding polypeptide to the binding partner modulates signal transduction in the cell;
wherein at least three, at least four, at least five, at least six, at least seven, at least eight, at least nine, at least ten, at least eleven or twelve of the binding polypeptides bind to and modulate signal transduction of the same binding partner; and
wherein the binding molecule can induce or inhibit signal transduction in the cell at a higher potency than an equivalent amount of a monovalent or divalent binding molecule with one or two binding polypeptides binding to the same binding partner.

2. A multimeric binding molecule comprising two, five, or six bivalent binding units or variants or fragments thereof,
wherein each binding unit comprises two IgA or IgM heavy chain constant regions or multimerizing fragments or variants thereof, each fused to binding polypeptide, wherein at least three of the binding polypeptides comprise a receptor ectodomain that specifically binds to a binding partner comprising a ligand or receptor-binding fragment thereof, wherein the receptor ectodomain is not an antibody or antigen-binding fragment of an antibody, and wherein binding of the receptor ectodomain to the ligand can modulate signal transduction in a cell that expresses the receptor;
wherein at least three, at least four, at least five, at least six, at least seven, at least eight, at least nine, at least ten, at least eleven or twelve of the receptor ectodomains bind to the same ligand; and
wherein the binding molecule can modulate signal transduction at a higher potency than an equivalent amount of a monomeric or dimeric binding molecule with one or two receptor ectodomains binding to the same ligand.

3. The multimeric binding molecule of claim 1 or claim 2, wherein each binding unit comprises two IgA heavy chain constant regions or multimerizing fragments or variants thereof, each comprising an IgA Cα3 domain and an IgA tailpiece domain, and wherein the multimeric binding molecule further comprises a J-chain or functional fragment or variant thereof.

4. The multimeric binding molecule of claim 3, wherein each IgA heavy chain constant region or multimerizing fragment or variant thereof further comprises an IgA Cα2 domain situated N-terminal to the IgA Cα3 and IgA tailpiece domains.

5. The multimeric binding molecule of claim 4, comprising amino acids 125 to 353 of SEQ ID NO: 24, or amino acids 113 to 340 of SEQ ID NO: 25.

6. The multimeric binding molecule of claim 4 or claim 5, wherein each IgA heavy chain constant region or multimerizing fragment or variant thereof further comprises an IgA hinge region situated N-terminal to the IgA Cα2 domain.

7. The multimeric binding molecule of claim 6, comprising amino acids 102 to 353 of SEQ ID NO: 24, or amino acids 102 to 340 of SEQ ID NO: 25.

8. The multimeric binding molecule of claim 1 or claim 2, wherein each binding unit comprises two IgM heavy chain constant regions or multimerizing fragments or variants thereof, each comprising an IgM Cμ4 domain and an IgM tailpiece domain.

9. The multimeric binding molecule of claim 8, wherein each IgM heavy chain constant region or multimerizing fragment or variant thereof further comprises an IgM Cμ3 domain situated N-terminal to the IgM Cμ4 and IgM tailpiece domains.

10. The multimeric binding molecule of claim 9, wherein each IgM heavy chain constant region or multimerizing fragment or variant thereof further comprises an IgM Cμ2 domain situated N-terminal to the IgM Cμ3 domain.

11. The multimeric binding molecule of claim 10, comprising a multimerizing fragment of the human IgM constant region comprising SEQ ID NO: 3.

12. The multimeric binding molecule of claim 10, comprising a multimerizing variant fragment of the human IgM constant region comprising SEQ ID NO: 4, wherein the multimeric binding molecule has reduced complement-dependent cytotoxicity (CDC) activity relative to a corresponding binding molecule comprising the wild type multimerizing fragment of the human IgM constant region of SEQ ID NO: 3.

13. The multimeric binding molecule of claim 9, wherein each IgM heavy chain constant region or multimerizing fragment or variant thereof further comprises an IgG hinge region or functional variant thereof situated N-terminal to the IgM Cμ3 domain.

14. The multimeric binding molecule of claim 13, comprising a variant human IgG1 hinge region fused to a multimerizing fragment of the human IgM constant region comprising the Cμ3, Cμ4, and TP domains, wherein the multimerizing hinge-IgM constant region fragment comprises SEQ ID NO: 6.

15. The multimeric binding molecule of claim 13, comprising a variant human IgG1 hinge region fused to a multimerizing fragment of the human IgM constant region comprising the Cμ3, Cμ4, and TP domains, wherein the multimerizing hinge-IgM constant region fragment comprises SEQ ID NO: 7, and wherein the multimeric binding molecule has reduced CDC activity relative to a corresponding binding molecule comprising the multimerizing hinge-IgM fragment of SEQ ID NO: 6.

16. The multimeric binding molecule of any one of claims 8 to 15 which is pentameric, and further comprises a J-chain or functional fragment or variant thereof.

17. The multimeric binding molecule of claim 16, wherein the J-chain or functional fragment or variant thereof is a variant J-chain comprising one or more single amino acid substitutions, deletions, or insertions relative to a wild-type J-chain that can affect serum half-life of the multimeric binding molecule; and wherein the multimeric binding molecule exhibits an increased serum half-life upon administration to an animal relative to a reference multimeric binding molecule that is identical except for the one or more single amino acid substitutions, deletions, or insertions, and is administered in the same way to the same animal species.

18. The multimeric binding molecule of claim 17, wherein the J-chain or functional fragment thereof comprises an amino acid substitution at the amino acid position corresponding to amino acid Y102 of the wild-type human J-chain (SEQ ID NO: 15).

19. The multimeric binding molecule of claim 18, wherein the amino acid corresponding to Y102 of SEQ ID NO: 15 is substituted with alanine (A), serine (S), or arginine (R).

20. The multimeric binding molecule of claim 19, wherein the amino acid corresponding to Y102 of SEQ ID NO: 15 is substituted with alanine (A).

21. The multimeric binding molecule of claim 20, wherein the J-chain is a variant human J-chain and comprises the amino acid sequence SEQ ID NO: 16.

22. The multimeric binding molecule of claim 17, wherein the J-chain or functional fragment thereof comprises an amino acid substitution at the amino acid position corresponding to amino acid N49, amino acid S51, or both N49 and S51 of the human J-chain (SEQ ID NO: 15), wherein a single amino acid substitution corresponding to position S51 of SEQ ID NO: 15 is not a threonine (T) substitution.

23. The multimeric binding molecule of claim 22, wherein the position corresponding to N49 of SEQ ID NO: 15 is substituted with alanine (A), glycine (G), threonine (T), serine (S) or aspartic acid (D).

24. The multimeric binding molecule of claim 23, wherein the position corresponding to N49 of SEQ ID NO: 15 is substituted with alanine (A).

25. The multimeric binding molecule of claim 24, wherein the J-chain is a variant human J-chain and comprises the amino acid sequence SEQ ID NO: 17.

26. The multimeric binding molecule of claim 22, wherein the position corresponding to S51 of SEQ ID NO: 15 is substituted with alanine (A) or glycine (G).

27. The multimeric binding molecule of claim 26, wherein the position corresponding to S51 of SEQ ID NO: 15 is substituted with alanine (A).

28. The multimeric binding molecule of claim 27, wherein the J-chain is a variant human J-chain and comprises the amino acid sequence SEQ ID NO: 18.

29. The multimeric binding molecule of any one of claims 3 to 7 or 16 to 28, wherein the J-chain or functional fragment or variant thereof further comprises a heterologous polypeptide, wherein the heterologous polypeptide is directly or indirectly fused to the J-chain or functional fragment or variant thereof.

30. The multimeric binding molecule of claim 29, wherein the heterologous polypeptide is fused to the J-chain or fragment thereof via a peptide linker.

31. The multimeric binding molecule of claim 30, wherein the peptide linker comprises at least 5 amino acids, but no more than 25 amino acids.

32. The multimeric binding molecule of claim 31, wherein the peptide linker consists of GGGGS (SEQ ID NO: 19), GGGGSGGGGS (SEQ ID NO: 20), GGGGSGGGGSGGGGS (SEQ ID NO: 21), GGGGSGGGGSGGGGSGGGGS (SEQ ID NO: 22), or GGGGSGGGGSGGGGSGGGGSGGGGS (SEQ ID NO: 23).

33. The multimeric binding molecule of any one of claims 29 to 32, wherein the heterologous polypeptide is fused to the N-terminus of the J-chain or fragment or variant thereof, the C-terminus of the J-chain or fragment or variant thereof, or to both the N-terminus and C-terminus of the J-chain or fragment or variant thereof.

34. The multimeric binding molecule of any one of claims 29 to 33, wherein the heterologous polypeptide can influence the absorption, distribution, metabolism and/or excretion (ADME) of the multimeric binding molecule.

35. The multimeric binding molecule of any one of claims 29 to 33, wherein the heterologous polypeptide comprises an antigen binding domain.

36. The multimeric binding molecule of claim 35, wherein the antigen binding domain of the heterologous polypeptide is an antibody or antigen-binding fragment thereof.

37. The multimeric binding molecule of claim 36, wherein the antigen-binding fragment comprises an Fab fragment, an Fab′ fragment, an F(ab′)2 fragment, an Fd fragment, an Fv fragment, a single-chain Fv (scFv) fragment, a disulfide-linked Fv (sdFv) fragment, or any combination thereof.

38. The multimeric binding molecule of claim 37, wherein the antigen-binding fragment is a scFv fragment.

39. The multimeric binding molecule of any one of claims 3 to 7, comprising four identical binding polypeptides.

40. The multimeric binding molecule of any one of claims 8 to 38, which is pentameric and comprises ten identical binding polypeptides.

41. The multimeric binding molecule of any one of claims 8 to 38, which is hexameric and comprises twelve identical binding polypeptides.

42. The multimeric binding molecule of any one of claims 1 or 3 to 41, wherein each binding polypeptide is a ligand or receptor-binding fragment thereof, a cytokine or receptor-binding fragment thereof, a growth factor or receptor binding fragment thereof, a neurotransmitter or receptor binding fragment thereof, a peptide or protein hormone or receptor binding fragment thereof, an immune checkpoint modulator ligand or receptor-binding fragment thereof, or a receptor-binding fragment of an extracellular matrix protein.

43. The multimeric binding molecule of claim 42, wherein the ligand or receptor-binding fragment thereof comprises a chemokine, a complement protein, a fibroblast growth factor (FGF) family ligand, an immune checkpoint modulator ligand, an epidermal growth factor (EGF), an interferon, a tumor necrosis factor superfamily (TNFSF) ligand, a vascular endothelial growth factor (VEGF) family ligand, a transforming growth factor-β superfamily (TGFβsf) ligand, any receptor-binding fragment thereof, or any combination thereof.

44. The multimeric binding molecule of claim 43, wherein the binding polypeptide comprises a TNFSF ligand, and wherein the TNFSF ligand comprises TRAIL, OX40 ligand, CD40 ligand, a glucocorticoid-induced tumor necrosis factor receptor ligand (GITRL), 4-1BB ligand, any receptor binding fragment thereof, or any combination thereof.

45. The multimeric binding molecule of claim 42, wherein the binding polypeptide comprises an immune checkpoint modulator ligand protein or receptor-binding fragment thereof and wherein the immune checkpoint modulator protein comprises CD86 or a receptor-binding fragment thereof, CD80 or a receptor-binding fragment thereof, PD-L1 or a receptor-binding fragment thereof, or any combination thereof.

46. The multimeric binding protein of claim 45, wherein the binding polypeptide comprises a receptor-binding fragment of human PD-L1.

47. The multimeric binding protein of claim 46, wherein the binding polypeptide comprises amino acids 19 to 127 of SEQ ID NO: 8, which contains the V-type domain of human PD-L1.

48. The multimeric binding protein of claim 47, wherein the binding polypeptide comprises SEQ ID NO: 9, which contains the V-type and C2-type domains of human PD-L1.

49. The multimeric binding molecule of claim 48, comprising ten or twelve copies of a polypeptide comprising the amino acid sequence SEQ ID NO: 11 or SEQ ID NO: 13.

50. The multimeric binding molecule of claim 49, further comprising a variant J-chain comprising the amino acid sequence SEQ ID NO: 16.

51. The multimeric binding molecule of any one of claims 46 to 50, which is an agonist of PD-1.

52. The multimeric binding molecule of any one of claims 1 or 3 to 51, wherein the binding partner is cell-surface receptor protein or an immune checkpoint modulator.

53. The multimeric binding molecule of any one of claims 2 to 41, wherein the receptor ectodomain comprises a ligand-binding fragment of a tumor necrosis factor superfamily receptor (TNFrSF), a ligand-binding fragment of an immune checkpoint modulator receptor, ligand-binding fragment of a TGFβ receptor, or any combination thereof.

54. The multimeric binding molecule of claim 53, wherein the TNFrSF receptor fragment comprises a ligand-binding fragment of death domain containing receptor-4 (DR4), death domain containing receptor-5 (DR5), OX-40, CD40, 4-1BB, glucocorticoid-induced tumor necrosis factor receptor (GITR), or any combination thereof.

55. The multimeric binding molecule of claim 53, wherein the immune checkpoint modulator receptor ectodomain comprises a ligand-binding fragment of PD-1, a ligand-binding fragment of CTLA4, a ligand-binding fragment of LAG3, a ligand-binding fragment of CD28, a ligand-binding fragment of immunoglobulin-like domain containing receptor 2 (ILDR2), a ligand-binding fragment of T-cell immunoglobulin mucin family member 3 (TIM-3), or any combination thereof.

56. The multimeric binding molecule of claim 53, wherein the TGFβ receptor comprises a TGFβR-1, a TGFβR-2, a TGFβR3, or any combination thereof.

57. An isolated polynucleotide comprising a nucleic acid sequence that encodes a subunit of the multimeric binding molecule of any one of claims 1 to 56, wherein each subunit comprises an IgA or IgM heavy chain constant region or multimerizing fragment or variant thereof fused to a binding polypeptide or fragment thereof that specifically binds to a binding partner, or a receptor ectodomain that specifically binds to a ligand.

58. A vector comprising the polynucleotide of claim 57.

59. A host cell comprising the vector of claim 58.

60. The host cell of claim 59, further comprising an isolated polynucleotide comprising a nucleic acid sequence encoding the J-chain or functional fragment or variant thereof of any one of claims 16 to 38.

61. A method for treating an autoimmune disorder, an inflammatory disorder, or a combination thereof in a subject in need of treatment comprising administering to the subject an effective amount of the multimeric binding molecule of any one of claims 45 to 55, wherein the multimeric binding molecule exhibits greater potency than an equivalent amount of a monomeric or dimeric binding molecule binding to the same binding partner.

62. A method for preventing transplantation rejection in a transplantation recipient, comprising administering to the subject an effective amount of the multimeric binding molecule of any one of claims 45 to 55, wherein the multimeric binding molecule exhibits greater potency than an equivalent amount of a monomeric or dimeric binding molecule binding to the same binding partner.


SEQUENCE LISTING (60 sequences)

SEQ ID NO: 1 - protein, 453 aa, Homo sapiens [sequence omitted]

SEQ ID NO: 2 - protein, 453 aa, Artificial Sequence
  Description of Artificial Sequence Synthetic polypeptide
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLASDLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 3 - protein, 349 aa, Homo sapiens
VIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 4 - protein, 349 aa, Artificial Sequence
  Description of Artificial Sequence Synthetic polypeptide
VIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 5 - protein, 18 aa, Homo sapiens
VEPKSSDKTHTCPPCPAP

SEQ ID NO: 6 - protein, 255 aa, Homo sapiens
VEPKSSDKTHTCPPCPAPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 7 - protein, 255 aa, Artificial Sequence
  Description of Artificial Sequence Synthetic polypeptide
VEPKSSDKTHTCPPCPAPDQDTAIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 8 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 9 - protein, 220 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 10 - protein, 587 aa, Artificial Sequence
  Description of Artificial Sequence Synthetic polypeptide
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 11 - protein, 587 aa, Artificial Sequence
  Description of Artificial Sequence Synthetic polypeptide
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 12 - protein, 475 aa, Artificial Sequence
  Description of Artificial Sequence Synthetic polypeptide
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERVEPKSSDKTHTCPPCPAPDQDTAIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 13 - protein, 493 aa, Artificial Sequence
  Description of Artificial Sequence Synthetic polypeptide
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERVEPKSSDKTHTCPPCPAPDQDTAIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 14 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 15 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 16 - protein, 137 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 17 - protein, 137 aa, Artificial Sequence
  Description of Artificial Sequence Synthetic polypeptide
QEDERIVLVDNKCKCARITSRIIRSSEDPNEDIVERNIRIIVPLNNREAISDPTSPLRTRFVYHLSDLCKKCDPTEVELDNQIVTATQSNICDEDSATETCYTYDRNKCYTAVVPLVYGGETKMVETALTPDACYPD

SEQ ID NO: 18 - protein, 137 aa, Artificial Sequence
  Description of Artificial Sequence Synthetic polypeptide
QEDERIVLVDNKCKCARITSRIIRSSEDPNEDIVERNIRIIVPLNNRENIADPTSPLRTRFVYHLSDLCKKCDPTEVELDNQIVTATQSNICDEDSATETCYTYDRNKCYTAVVPLVYGGETKMVETALTPDACYPD

SEQ ID NO: 19 - protein, 5 aa, Artificial Sequence
  Description of Artificial Sequence Synthetic peptide
GGGGS

SEQ ID NO: 20 - protein, 10 aa, Artificial Sequence
  Description of Artificial Sequence Synthetic peptide
GGGGSGGGGS

SEQ ID NO: 21 - protein, 15 aa, Artificial Sequence
  Description of Artificial Sequence Synthetic peptide
GGGGSGGGGSGGGGS

SEQ ID NO: 22 - protein, 20 aa, Artificial Sequence
  Description of Artificial Sequence Synthetic peptide
GGGGSGGGGSGGGGSGGGGS

SEQ ID NO: 23 - protein, 25 aa, Artificial Sequence
  Description of Artificial Sequence Synthetic peptide
GGGGSGGGGSGGGGSGGGGSGGGGS

SEQ ID NO: 24 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 25 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 26 - protein, 764 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 27 - protein, 585 aa, Homo sapiens
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPR

SEQ ID NO: 28 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 29 - protein, 205 aa, Homo sapiens
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 30 - protein, 244 aa, Homo sapiens
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 31 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 32 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 33 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 34 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 35 - protein, 281 aa, Homo sapiens [sequence omitted]

SEQ ID NO: 36 - protein, 199 aa, Homo sapiens
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 37 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 38 - protein, 329 aa, Homo sapiens
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 39 - protein, 468 aa, Homo sapiens
MAPPPARVHLGAFLAVTPNPGSAASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVHKESGNGHNIWVILVVTLVVPLLLVAVLIVCCCIGSGCGGDPKCMDRVCFWRLGLLRGPGAEDNAHNEILSNADSLSTFVSEQQMESQEPADLTGVTVQSPGEAQCLLGPAEAEGSQRRRLLVPANGADPTETLMLFFDKFANIVPFDSWDQLMRQLDLTKNEIDVVRAGTAGPGDALYAMLMKWVNKTGRNASIHTLLDALERMEERHAREKIQDLLVDSGKFIYLEDGTGSAVSLE

SEQ ID NO: 40 - protein, 440 aa, Homo sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 41 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 42 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 43 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 44 - protein, 241 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 45 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 46 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 47 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 48 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 49 - protein, 639 aa, Homo sapiens
MDRVLLRWISLFWLTAMVEGLQVTVPDKKKVAMLFQPTVLRCHFSTSSHQPAVVQWKFKSYCQDRMGESLGMSSTRAQSLSKRNLEWDPYLDCLDSRRTVRVVASKQGSTVTLGDFYRGREITIVHDADLQIGKLMWGDSGLYYCIITTPDDLEGKNEDSVELLVLGRTGLLADLLPSFAVEIMPEWVFVGLVLLGVFLFFVLVGICWCQCCPHSCCCYVRCPCCPDSCCCPQALYEAGKAAKAGYPPSVSGVPGPYSIPSVPLGGAPSSGMLMDKPHPPPLAPSDSTGGSHSVRKGYRIQADKERDSMKVLYYVEKELAQFDPARRMRGRYNNTISELSSLHEEDSNFRQSFHQMRSKQFPVSGDLESNPDYWSGVMGGSSGASRGPSAMEYNKEDRESFRHSQPRSKSEMLSRKNFATGVPAVSMDELAAFADSYGQRPRRADGNSHEARGGSRFERSESRAHSGFYQDDSLEEYYGQRSRSREPLTDADRGWAFSPARRRPAEDAHLPRLVSRTPGTAPKYDHSYLGSARERQARPEGASRGGSLETPSKRSAQLGPRSASYYAWSPPGTYKAGSSQDDQEDASDDALPPYSELELTRGPSYRGRDLPYHSNSEKKRKKEPAKKTNDFPTRMSLVV

SEQ ID NO: 50 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 51 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 52 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 53 - protein, 851 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 54 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 55 - protein, 1363 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEHRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFSCKGPGQNVAVTRAHPDSQGRRRRPERGARGGQVFYNSEYGELSEPSEEDHCSPSARVTFFTDNSY

SEQ ID NO: 56 - protein, 1338 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLFIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 57 - protein, 431 aa, Artificial Sequence
  Description of Artificial Sequence Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 277 aa, Homo sapiens
MIILIYLFLLLWEDTQGWGFKDGIFHNSIWLERAAGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKECGGVFTDPKQIFKSPGFPNEYEDNQICYWHIRLKYGQRIHLSFLDFDLEDDPGCLADYVEIYDSYDDVHGFVGRYCGDELPDDIISTGNVMTLKFLSDASVTAGGFQIKYVAMDPVSKSSQGKNTSTTSTGNKNFLAGRFSHL

SEQ ID NO: 59 - protein, 9 aa, Unknown
  Description of Unknown Synovial endothelium targeting peptide
CKSTHDRLC

SEQ ID NO: 60 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY